(19) United States
(12) Patent Application Publication
(10) Pub. No.: US 2026/0090753 A1
(43) Pub. Date: Apr. 2, 2026

(54) System and Apparatus for Wearable Hemodynamic Monitoring

(71) Applicant: HEMODYNAMIQ WEARABLES PRIVATE (PVT) LIMITED (LTD), Bangalore (IN)

(72) Inventors: Deepanjan DATTA, Bangalore (IN); Sai Kamalesh RAYAPROLU, Bangalore (IN)

(21) Appl. No.: 19/315,900

(22) Filed: Sep. 2, 2025

Related U.S. Application Data
(60) Provisional application No. 63/689,780, filed on Sep. 2, 2024.

(51) Int. Cl. A61B 5/282; A61B 5/00; A61B 5/0535; A61B 5/257; A61B 5/265; A61B 5/366

(52) U.S. Cl. CPC A61B 5/282; A61B 5/0535; A61B 5/257; A61B 5/265; A61B 5/366; A61B 5/725; A61B 5/7264; A61B 5/7275; A61B 2560/0228; A61B 2562/0215; A61B 2562/0219; A61B 2562/166; A61B 2562/227

(57) ABSTRACT

A wearable, non-invasive, and non-intrusive hemodynamic monitoring device is disclosed for continuous monitoring of left ventricular (LV) hemodynamic waveforms and associated cardiovascular parameters. The device includes a flexible body with laterally extending electrode wings and a central rigid housing containing signal acquisition and transmission electronics. In various embodiments, the device includes either two or three wings configured for placement along the midsternal region and over the carotid arteries to enable simultaneous acquisition of electrical and mechanical signals via bioimpedance, ECG/EKG, and accelerometer sensors. Each wing incorporates tetrapolar bioimpedance electrode arrays, current injection electrodes, voltage sensing electrodes, and accelerometers to capture thoracic and cervical physiological signals necessary for estimating parameters such as left ventricular end diastolic pressure, extracellular fluid, left ventricular ejection fraction, cardiac out, stroke volume and QRS measures. The sensor signals are routed through flexible interconnects or magnetic mating connectors to the central housing. The system includes a wireless transmitter module supporting Bluetooth, Wi-Fi, or other wireless standards, a power supply and power management circuitry, and is constructed on ultra-thin, stretchable substrates composed of biocompatible flexible polymers. The modular design also includes user-serviceable magnetic connectors and optional adhesive layers for improved skin contact, thereby enabling continuous, real-time cardiovascular monitoring suitable for ambulatory and remote healthcare environments.

### CROSS-REFERENCE TO RELATED APPLICATION

This application claims priority to U.S. Provisional Patent Application Serial No. 63/689,780 filed on Sep. 2, 2024. This application also cross-references to concurrently filed U.S. patent application Ser. No. 17/833,908, titled “Wearable Hemodynamic Monitoring Device and System”, filed on Jun. 7, 2022. All disclosures are herein incorporated by reference in their entirety for all purposes.

### FIELD

The present invention is generally applicable to medical devices. More specifically, it pertains to developing a wearable, noninvasive, and nonintrusive monitoring device designed to assess hemodynamic parameters that may reduce the likelihood of adverse events in patients suffering from congestive heart failure or other associated cardiac conditions.

### BACKGROUND

Heart failure (HF) is a complex, multifactorial clinical syndrome associated with high morbidity and mortality, representing a significant societal and economic burden. It is estimated that more than 64 million individuals are affected by this disease worldwide, with its prevalence projected to increase by 46% over the next two decades. In the United States, HF accounts for over a million hospital admissions annually, ranking it among the most expensive health conditions to manage. Historically, heart failure has been linked to reduced ejection fraction (HFrEF), but the predominant and fastest-growing form of the disease is heart failure with preserved ejection fraction (HFpEF), which also severely impacts physical capacity and quality of life (QoL). In contrast to HFrEF, clinical trials of pharmacological treatments for HFpEF have largely failed to improve patient outcomes, leaving few effective therapeutic options available. Consequently, there is a critical need for effective strategies to improve physical capacity and QoL for patients with HFpEF.

The early post-discharge period, known as the “vulnerable phase,” begins with an acute heart failure (AHF) incident and may last up to six months after leaving the hospital. Patients who successfully navigate this phase often develop a more stable clinical condition, marking the end of the vulnerable phase. During this period, the likelihood of readmission and mortality is significantly high, with rates reaching 30% and 10%, respectively, within the first few weeks. Contributing factors to readmissions during this phase include patient-related issues, such as non-adherence to medications and dietary indiscretions, as well as system-related problems, including insufficient follow-up care after discharge. In the subsequent six months after discharge, factors like heart attacks, atrial fibrillation, uncontrolled hypertension, diabetes, COPD, and infections can lead to readmissions and mortality.

Nevertheless, the risk of readmission and mortality gradually decreases over time. Mortality risk decreases from six times higher in the first month after discharge to twice as high during the vulnerable phase. Therefore, proactive monitoring of heart failure events (HFE) during this vulnerable phase can enhance patient outcomes by altering the typical course of the disease prognosis.

Patients with acute heart failure (AHF) show considerable variability, yet certain factors are linked to the onset of the vulnerable phase. Failure to alleviate congestion during the initial hospital stay may lead to a short-term deterioration of the heart's pumping ability, resulting in increased pressure in the left ventricle (LV) and capillary vessels. This increase in pressure indicates poor LV function, mainly in patients with HFpEF. Persistent high pressure can lead to long-term damage to various organs, as evidenced by abnormal levels of troponin and creatinine. Consequently, it is essential to identify patients at risk of poor post-discharge outcomes and to address congestion before discharging AHF patients.

HFpEF is a highly heterogeneous syndrome, meaning patients can be divided into different “congestion phenotypes” based on clinical, hemodynamic, and biomarker profiles. Central to defining these subgroups in HFpEF is the measurement of intracardiac filling pressure (such as PAP, PCWP, LVEDP) and assessment of extravascular (interstitial) fluid content (ECF), as both indicate congestion and disease severity. PAP: pulmonary artery pressure, PCWP: pulmonary capillary wedge pressure, LVEDP: left ventricular end-diastolic pressure. Elevated filling pressure is a hallmark of congestion in HFpEF; studies consistently show that LVEDP values exceeding 22 mmHg are associated with significantly higher in-hospital mortality rates. The prognostic power of LVEDP extends far beyond acute hospital settings. Research involving over 6,735 consecutive patients undergoing coronary artery bypass surgery revealed that elevated LVEDP serves as an independent predictor of long-term mortality, particularly in patients with HFpEF. In contrast, extracellular (interstitial) fluid, assessed as part of total body water or lung water, reflects systemic or pulmonary congestion, contributing to symptoms and risk stratification. The extracellular-to-total body water ratio (ECW/TBW) emerges as a potent biomarker, with elevated ratios above 0.4 associated with nearly six-fold increased mortality risk in vulnerable populations.

Congestion in HF is not monolithic. For example, there are multiple simultaneous congestion phenotypes, such as pulmonary tissue congestion with dilated left ventricle, pulmonary tissue congestion in HFpEF, mixed pulmonary-systemic congestion with atrial fibrillation, pulmonary intravascular congestion with chamber dilatation, and global congestion. These phenotypes can coexist in HF patients, and their dominant form may shift over time-even within a single hospital admission. For example, a HFpEF patient may exhibit both pulmonary intravascular congestion (e.g., increases in PAP/PCWP/LVEDP) and pulmonary tissue congestion (e.g., increases in ECF), each requiring distinct management strategies. Therefore, we recognized that simultaneous identification of multiple congestion phenotypes in HF is essential for precision management, given the complex interplay of hemodynamic factors, body fluid distribution, and comorbidities.

Early identification of increased filling pressure is crucial for reducing readmissions in HF patients. Recent studies have shown that remote monitoring devices can lower readmission rates and improve outcomes for HF patients by tracking the biomarkers of worsening heart failure events before clinical symptoms appear.

Conventionally, implantable pressure sensor devices for monitoring pulmonary artery pressure (PAP) are recommended and used as a means to reduce hospitalizations associated with heart failure. Although such devices provide a gold standard assessment for intravascular congestion, their widespread clinical use remains constrained by technical complexity, high costs, limited accessibility, and potential procedural complications, including infection, hematoma, and arrhythmia. For instance, despite evidence showing a substantial decline in hospital readmissions (e.g., with reductions of approximately 37% at six months and about 62% at twelve months), the utilization of the CardioMEMS implantable PAP monitoring device from Abbott Inc. has persisted at less than 2% worldwide between 2014 and 2024. Furthermore, in acute care settings, the application of pulmonary artery catheterization for guiding decongestive therapy did not yield significant improvements in overall mortality or hospitalization rates, as highlighted by the ESCAPE (Evaluation Study of Congestive Heart Failure and Pulmonary Artery Catheterization Effectiveness) trial. In addition, implantable devices are invasive.

As described, such conventional devices do not directly measure different types of congestion, such as pulmonary intravascular congestion and tissue congestion. Furthermore, such conventional devices require implantation into the body.

From the foregoing discussion, it is desirable to provide a comprehensive cardiovascular non-invasive monitoring system for long-term monitoring of HF patients, which is capable of directly measuring and simultaneously assessing both pulmonary intravascular congestion and tissue congestion.

### BRIEF SUMMARY

A wearable hemodynamic monitoring device is disclosed wherein physiological parameters are tracked through integrated bioimpedance sensing, accelerometry, and electrocardiographic (ECG/EKG) waveform analysis, the device being configured for comfortable and non-intrusive placement on a user's body during either brief daily monitoring periods or continuous extended wear without removal; heart failure (HF) symptomatology is evaluated through variant embodiments configured to provide precise clinical assessment while simultaneously monitoring additional physiological indicators, thereby enabling prediction of decompensation events, initiation of proactive therapeutic interventions, and reduction of manufacturing costs through optimized system architecture.

In one embodiment, a wearable device for monitoring hemodynamic waveforms in a user is disclosed, wherein the device includes a flexible body having at least three flexible bioimpedance measuring wings extending laterally from a rigid housing, the flexible wings being fabricated from a first set of materials configured to conform to the user's body surface while the rigid housing is constructed from a second set of materials providing structural integrity and component protection. An array of flexible electrodes and sensor elements is embedded within each wing, the electrodes and sensors being configured to establish conformal contact with the user's surface for detection of bioimpedance waveforms, electrocardiographic (ECG/EKG) signals, and acceleration waveforms representative of user physiological parameters. A plurality of conductive traces is embedded within the wings and mechanically decoupled from the rigid housing; the conductive traces being configured to maintain conformal surface contact while transmitting electrical signals from the electrodes and sensors to a signal acquisition unit (SAU) positioned within the rigid housing. The SAU is implemented as a printed circuit board (PCB)-based electronics assembly enclosed within the rigid housing and includes a plurality of bioimpedance analog front-end (AFE) circuits configured to generate and inject excitation current pulses into the user's body via designated electrodes at predetermined intervals, and a plurality of electrocardiographic AFE circuits configured to measure cardiac electrical activity through designated electrodes and digitize the signals to generate ECG/EKG waveforms. A processor is integrated within the SAU and configured to capture and convert signals from the electrode and sensor array into machine-readable digitized data through analog-to-digital (ADC) conversion processes. A memory unit is provided and configured to store operational software for processor control and to retain acquired data including signals received from the electrode and sensor array. A wireless transmitter module is incorporated and configured to transmit preprocessed data from the processor to external devices including smartphones and web servers via wireless communication protocols including Bluetooth, Wi-Fi, or alternative wireless standards. A power supply is integrated within the device and configured to provide electrical power to the analog front-end circuits, processor, memory unit, and wireless transmitter module, thereby enabling autonomous operation of the hemodynamic monitoring system.

In another embodiment, a method for monitoring a patient with congestive heart failure (CHF) was disclosed, wherein a wearable, non-invasive, and non-intrusive hemodynamic monitoring patch classified as a Class II medical device was provided for continuous or periodic application to a patient diagnosed with heart failure. The wearable monitoring device includes a central rigid housing and at least three flexible bioimpedance-measuring wings extending laterally therefrom, each wing adapted for anatomical placement to enable hemodynamic signal acquisition. In the described configuration, a first bioimpedance-measuring wing is configured for placement along the midsternal region of the user's upper thorax, a second bioimpedance-measuring wing is configured for placement over the right side of the neck at the location of the right common carotid artery (RCCA), and a third bioimpedance-measuring wing is configured to be positioned on the left side of the neck over the left common carotid artery (LCCA). Each of the flexible wings is fabricated from biocompatible, conformable materials selected to ensure proper skin contact and long-term user comfort while enabling precise sensor positioning across relevant vascular sites. Each of these wings further includes an array of electrodes and sensor elements. In the exemplary configuration, the array in the first wing includes a first and a second electrode disposed along the midsternal line near the upper and lower ends of the wing, respectively, the electrodes being configured to inject electrical current into the upper thoracic region of the user's body for the purpose of impedance-based hemodynamic measurement. In addition, a third and a fourth electrode are positioned longitudinally below the positive current-injecting electrode along the same midline, with the third electrode configured as an initial voltage pickup point and the fourth electrode situated below the third to allow for the measurement of the potential difference between the two, thus enabling real-time acquisition of a first set of bioimpedance waveform under resting or ambulatory conditions. Likewise, a fifth and a sixth electrode are positioned longitudinally below the voltage detecting electrodes along the same midline, with the fifth electrode configured as an initial voltage pickup point and the sixth electrode situated below the fifth to allow for the measurement of the potential difference between the two, enabling real-time acquisition of a second set of bioimpedance waveform. Both the arrays in the second and third bioimpedance-measuring wings include a first and a second electrode disposed along the carotid arteries near the upper and lower ends of the wing, respectively, the electrodes being configured to inject electrical current into the carotid artery of the user's body. The corresponding voltage signal is sensed across a set of electrode pairs along the carotid sites. As the carotid artery undergoes pulsatile expansion and contraction with each cardiac cycle, local changes in vascular volume and blood flow cause characteristic fluctuations in the measured impedance waveform. These dynamic impedance changes are detected by the wing's electrodes and processed to yield beat-to-beat information such as arterial pulse arrival time, pulse wave velocity, and relative estimates of arterial pressure and compliance. This enables the device to non-invasively capture high-fidelity arterial pulse waveforms from both sides of the neck, supporting central blood pressure estimation, pulse transit time calculation, and detection of asymmetries or anomalies pertinent to cardiovascular risk assessment and patient monitoring.

In a further embodiment, a backend analysis system for predictive monitoring of a patient diagnosed with congestive heart failure (CHF) was disclosed, wherein a customized baseline model is generated using waveform data acquired from a wearable patch configured for hemodynamic monitoring. The system is configured to predict waveform behavior several days in advance of a potential hospitalization event and is further adapted to estimate a set of prospective left ventricular (LV) hemodynamic parameters critical for both HFrEF and HFpEF patient subgroups. The set of LV hemodynamic parameters include a chest-based pulse transit time derived from bioimpedance waveforms collected via a first array of electrodes disposed along the midsternal line on a first bioimpedance-measuring wing. Additionally, right and left neck-based pulse transit times are derived from bioimpedance signals acquired from a second and third bioimpedance-measuring wing, respectively, wherein the electrodes were positioned over the right common carotid artery (RCCA) and the left common carotid artery (LCCA). Stroke volume (SV) and cardiac output (CO) are calculated based on thoracic bioimpedance waveforms obtained through the electrode array associated with the first wing. Left ventricular ejection fraction (LVEF) is determined by extracting the pre-ejection period (PEP) and the left ventricular ejection time (LVET) from electrocardiographic (ECG/EKG) waveform analysis. Heart rate (HR) and heart rate variability (HRV) are extracted concurrently from the same ECG/EKG waveform data. The extracellular fluid (ECF) volume is quantified using the Cole-Cole impedance parameter R₀, which is computed through a time-domain fitting approach based on a single-frequency, DC-biased sinusoidal excitation signal. Left ventricular end-diastolic pressure (LVEDP) is estimated non-invasively from bioimpedance measurements by analyzing changes in thoracic impedance and vascular compliance across the cardiac cycle. The extracted impedance and ECG-derived timing features are processed using computational models that correlate changes in bioimpedance signals, reflective of left ventricular filling dynamics, to established LVEDP surrogates, enabling continuous and non-invasive estimation of LVEDP from wearable patch data. This non-invasively derived set of predictive parameters is configured to enable early detection of impending heart failure decompensation, thereby facilitating timely clinical intervention and reducing risk of acute hospitalization.

In another embodiment, a wearable hemodynamic monitoring device is disclosed, wherein the device includes a flexible body including at least three bioimpedance-measuring wings. The first wing is configured for midsternal placement and includes multiple electrodes arranged to inject alternating current and sense voltages at different locations on the midsternal line to measure thoracic impedance waveforms and pulse transit time (PTT). However, the second and third wings, positioned over the right and left common carotid arteries, are each configured with a one set of voltage-sensing electrodes for acquisition of carotid bioimpedance waveforms. The measured impedance waveforms from both the neck wings are processed using signal acquisition circuitry and a neural network processing unit. The processor is configured to extract physiological features from each cardiac cycle-including beat-to-beat bioimpedance amplitude changes, systolic/diastolic slope differentials, and inverse time delays between forward and reflected pressure waves in order to estimate pulse pressure and pulse wave velocity as indicators of vascular health. Carotid pressure values, including systolic, diastolic, and pulse pressure, are estimated using a physics-constrained neural network model incorporating Taylor approximation terms and trained with a dual loss function combining conventional supervised metrics and physics-based residuals. The machine learning architecture may include a two-layer 1D convolutional neural network followed by pooling, flattening, and fully connected layers, optimized for processing waveforms sampled at 30 Hz. Signal pre-processing includes second-order low-pass Butterworth filtering at 6 Hz, automatic cardiac cycle boundary detection via derivative waveform analysis, and extraction of nine cardiac fiducial points per cycle. Carotid placement is selected due to its anatomical proximity to the aortic arch and favorable signal quality for non-invasive central arterial waveform collection, enabling robust cardiovascular health assessment through wearable sensing.

In a separate embodiment, a chest-worn wearable hemodynamic monitoring device is disclosed, wherein pulsatile activity of the aorta and adjacent arteries is monitored using a high-density bioimpedance (BioZ) electrode array integrated within a flexible body configured for anatomical conformity. The device includes a conformable flexible body attached to a central rigid housing containing a signal acquisition unit (SAU), the two sections being connected via magnetic Pogo pin connectors for robust electrical interfacing. The flexible body incorporates a M×N array (M=6, N≥3) with at least eighteen bioimpedance electrodes arranged in three vertical columns, each configured for tetrapolar impedance measurements, as well as three electrocardiographic (ECG/EKG) electrodes and a three-axis accelerometer. The flexible substrate and embedded traces are constructed using stacked stretchable materials including elastomeric textile layers, conductive silver and silver chloride inks, and dielectric isolation layers to maintain signal integrity under mechanical deformation. Multiple analog front-end (AFE) circuits, enclosed in the rigid housing, are configured for synchronous current injection and voltage detection across electrode columns, along with ECG signal acquisition, ADC conversion, and wireless communication modules. The SAU supports independent multi-channel impedance measurements and synchronized ECG acquisition, coordinated through a processor configured with embedded firmware for signal processing, device control, and data transmission. The architecture enables continuous, non-invasive monitoring of aortic waveform parameters including multi-point thoracic impedance, PTT, PEP and LVET for cardiovascular assessment. A modular packaging method with a multi-level exploded assembly is employed to facilitate efficient manufacturing, accurate component alignment, and long-term device reliability under prolonged clinical or home use conditions.

In another embodiment, a wearable hemodynamic monitoring device is disclosed. The device includes a rigid housing with a signal acquisition unit (SAU). The SAU is configured with a data transmission module and a processing module. The device also includes a flexible body which includes bioimpedance and ECG/EKG electrodes configured to acquire raw EKG/ECG signals from a subject wearing the hemodynamic monitoring device. The flexible body is connected to the rigid housing and are in electrical communication with the SAU. The processing module of the SAU preprocesses the raw EKG/ECG acquired from the subject to produce preprocessed EKG/ECG signals. The data transmission module transmits the preprocessed EKG/ECG signals to a backend system for analysis and the wearable hemodynamic monitoring device facilitates predicting heart failure based on multiple simultaneous congestion phenotypes.

These and other aspects and embodiments of the invention are described in greater detail below, with reference to the drawing figures.

### BRIEF DESCRIPTION OF THE DRAWING

The accompanying drawings, which are incorporated in and form part of the specification, illustrate preferred embodiments of the present disclosure. The drawings, together with the description, serve to explain the principles of various embodiments of the present disclosure. Also, it is understood that the drawings may not necessarily be drawn to scale.

FIG. 1 shows a simplified embodiment of an exemplary wearable hemodynamic monitoring patch-based system for remotely monitoring heart failure patients;

FIGS. 2a and 2b show 3D perspective and partially exploded views, respectively, of an embodiment of a wearable hemodynamic monitoring patch;

FIGS. 2c and 2d show 3D exploded views of an embodiment of a flexible body as well as a wearable hemodynamic monitoring patch, which includes a number of optional items;

FIG. 3 shows an exemplary embodiment of a block diagram for the signal acquisition unit (SAU) corresponding to the embodiment in FIGS. 2a-2d;

FIG. 4 shows an exemplary embodiment of a process flow diagram illustrating a process to calculate LV hemodynamic parameters such as left ventricular end diastolic pressure LVEDP, extracellular fluid ECF, left ventricular ejection fraction LVEF and stroke volume SV from the data measured from the wearable device described in FIGS. 2a-2d;

FIGS. 5a and 5b show 3D perspective and exploded views, respectively, of an embodiment of a wearable hemodynamic monitoring patch, according to a separate embodiment;

FIGS. 5c and 5d show 3D exploded views of a chest bioimpedance wing and a neck bioimpedance wing of a wearable hemodynamic monitoring patch described in FIGS. 5a-5b;

FIG. 5ₑ shows 3D exploded view of a wearable hemodynamic monitoring patch, which includes a number of optional items, as described in FIGS. 5a-5d;

FIG. 6 shows an exemplary block diagram of an embodiment of a signal acquisition unit (SAU);

FIG. 7 shows an exemplary embodiment of a process flow diagram illustrating a neural network (NN) model to calculate left ventricular end diastolic pressure LVEDP, along with a process to calculate extracellular fluid ECF, left ventricular ejection fraction LVEF and stroke volume SV from the data measured from the wearable device described in FIGS. 5a-5d;

FIGS. 8a and 8b show 3D perspective views of another embodiment of a chest-worn wearable hemodynamic monitoring device;

FIG. 8c shows a 2D view of an embodiment of an electrode array embedded into a flexible body of a chest-worn wearable hemodynamic monitoring device;

FIG. 8d shows a 3D partially exploded view of a chest-worn wearable hemodynamic monitoring patch described in FIGS. 8a-8c;

FIG. 8e shows a 3D exploded view of a chest-worn hemodynamic monitoring patch as described in FIGS. 8a-8d;

FIG. 9 shows an exemplary embodiment of a process flow diagram illustrating a process to calculate LV hemodynamic parameters such as left ventricular end diastolic pressure LVEDP, extracellular fluid ECF and left ventricular ejection fraction LVEF from the data measured from the wearable device described in FIGS. 8a-8e;

FIG. 10a shows a 3D perspective view of a wearable hemodynamic monitoring patch, according to a separate embodiment;

FIG. 10b shows a 2D cross-sectional view of a wearable hemodynamic monitoring patch described in FIG. 10a along line A-A, illustrating the structural integration between the rigid circular housing and the bioimpedance wings;

FIGS. 10c and 10d show 3D exploded views of a chest bioimpedance wing and a neck bioimpedance wing of a wearable hemodynamic monitoring patch described in FIGS. 10a-10b;

FIG. 10ₑ shows a 3D exploded view of a flexible body as well as of a wearable hemodynamic monitoring patch as described in FIGS. 10a-10d;

### DETAILED DESCRIPTION

The subsequent explanation pertains to several diverse embodiments. However, the embodiments described can be implemented and modified in various ways without exceeding the boundaries of the invention. For instance, the described embodiments can be applied in any appropriate device, apparatus, or system to observe various physiological parameters. Specifically, the subsequent conversation primarily centers around long-term, patch-based hemodynamic monitoring devices to characterize patients suffering from heart failure with reduced ejection fraction (HFrEF) or heart failure with preserved ejection fraction (HFpEF) and other related diseases. For example, wearable devices, systems, and methods help monitor patients with congestive heart failure (CHF) and other severe left ventricular (LV) systolic and diastolic dysfunctions.

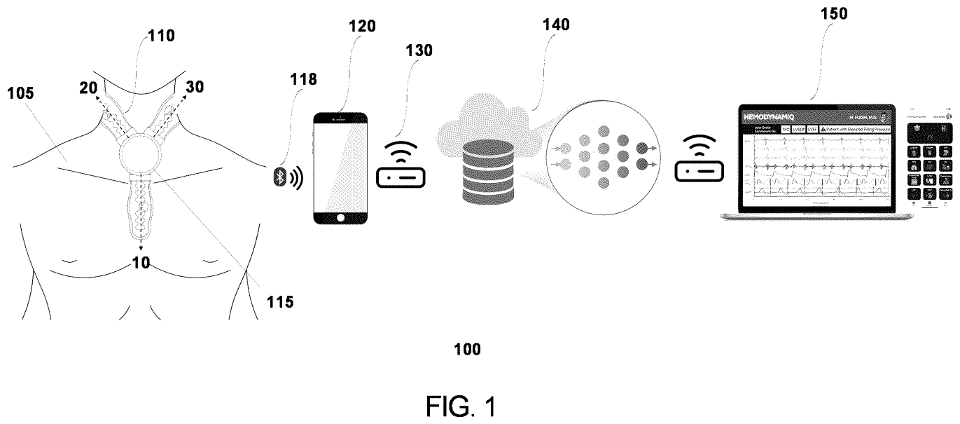

FIG. 1 shows an embodiment of a system 100 for monitoring patients. As shown, the system includes a wearable hemodynamic monitoring device 110. The wearable device, for example, is a patch worn on the upper part of a patient's chest (user) 105. The patch is designed to be worn continuously and comfortably for a short period (such as a few hours to a few days) or longer (up to several weeks). The wearable device may be configured with wet or dry conductive electrodes for current injection and voltage detection and Internet of Things (IoT) electronics, which include bioimpedance and ECG/EKG electrodes. In addition, the wearable device includes accelerometer sensors to detect motion signals in three axes for compensation of the signals affected by the patient's movements. Other types of sensors may also be included.

Upon wearing the wearable device 110, the electrodes and sensors are placed at specific locations on the body 105. The electrodes acquire raw physiological signals and electrically transmit them to the signal acquisition unit (SAU) 175. The SAU is configured to receive the acquired data, which includes ECG/EKG and bioimpedance waveforms, as well as 3-axis motion signals. Receiving other signals by the SAU may also be useful, depending on the type of sensors. Concerning the thoracic bioimpedance signals, they are processed, separating them into cardiac and respiratory components. The SAU transmits the waveforms (pre-processed waveforms) to the IoT gateway, such as a user's mobile device 120, via a short-range wireless transmitter module 118. Other types of gateways, including transmission techniques, may also be useful.

The user's mobile device may be, for example, a smartphone or a tablet computer, which may run on any platform, such as Android IOS or Windows. Other types of mobile devices or platforms may also be helpful. For example, the mobile device may be a dedicated device for receiving the pre-processed waveforms from the signal acquisition unit of the wearable device. The short-range wireless transmitter module may be a Bluetooth, Wi-Fi or other wireless communication standard. Different types of short-range wireless transmitter modules may also be helpful.

The pre-processed data is then sent to a web server 140 via Wi-Fi or cellular systems 130 for analysis using deep learning-based models. These models may employ a cascaded or ensemble architecture to handle the diversity of waveform data effectively. Other types of models may also be beneficial. In one embodiment, the server is a cloud-based web server, although different configurations may also apply. The deep learning models are specifically trained to account for each user's baseline characteristics. Given the variations in baseline characteristics of hemodynamic waveforms from one user to another, this approach is effective for accurate remote patient monitoring (RPM).

The waveforms obtained using the patch worn by the heart failure patient who is either relatively stable at home after the discharge from a hospital or post-emergency department (ED) visit or currently visiting skilled nursing facilities (SNF) or under observation by home health agencies (HHA) are used to predict the next few days of the waveforms using deep learning algorithms. These deep learning algorithms would take several days of data as input and predict waveforms that the patient is supposed to develop over the next few days as the output. These predicted waveforms could include irregular heart rhythm, variation in thoracic fluid volume, or changes in left ventricular pressure. T₀ achieve this, deep learning algorithms would require preprocessing, such as noise removal, feature extraction, missing value interpolation, and other preprocesses. Depending on the diversity of the waveforms' characteristics, some of the deep learning algorithms may need to have a cascade or an ensemble architecture. Subsequently, the predicted waveforms are taken as inputs and fed into the Gaussian mixture model (GMM) to predict the probability of acute deterioration triggered by possible heart failure events.

The prediction model is configured to detect acute deterioration in heart failure patients based on LV filling pressure and extracellular fluid volume-guided hemodynamic congestion. In one embodiment, the deep learning model for predicting heart failure events (HFE) satisfies several criteria. First, it should be capable of addressing the time-series nature of EKG and hemodynamic waveforms. Second, it should be able to represent the diverse physical conditions of patients. Third, it should evaluate multiple types of hemodynamic waveforms simultaneously. Fourth, it should identify gradual abnormalities in predicted waveforms during the “vulnerable phase” from euvolemic to presymptomatic congestion.

In one embodiment, the model uses a hidden Markov model (HMM) integrated within the GMM framework to fulfil the first two criteria. This allows the model to represent various user HF events through the concepts of “states” and “probabilistic transitions” between these states. For instance, a patient's condition can be classified as “normal deterioration” or “acute deterioration,” with changes in EKG/ECG and hemodynamic waveforms influencing the transitions between these classifications. T₀ model complex hemodynamic waveforms and meet the third and fourth criteria, the prediction model combines a continuous HMM with a multi-dimensional GMM. This allows the probabilistic output of each HMM state to be expressed using a multi-dimensional mixed Gaussian distribution, facilitating the calculation of the likelihood of deterioration events based on various types of hemodynamic waveforms. The parameters of the HMM and GMM may be acquired through a log-linearization process under the neural network framework, which enables comprehensive training of the connection parameters. This capability makes the method effective and accurate for the probabilistic classification of time-dependent EKG/ECG and bioimpedance waveforms.

The predicted waveforms output the posterior probability of each class (normal or acute deterioration) via multidimensional GMM. The clinical parameters calculated using the predicted waveforms include (i) LV filling pressure—a measure of pulmonary intravascular congestion, and (ii) extracellular fluid volume—a measure of pulmonary tissue congestion. These predicted clinical parameters may be transmitted to the physician of the user, for example, displayed on the physician's dashboard. Additionally, the model incorporates an anomaly detection strategy that identifies patterns in the clinical parameters calculated using the predicted waveforms and triggers an alert for the patient to visit the physician. Therefore, these predicted clinical parameters can indicate whether the patient is likely to require hospitalization or not, enabling early detection of heart failure events and aiming to reduce hospitalizations by preventing patients from reaching a critical stage. This closed-loop involves obtaining hemodynamic waveforms, preprocessing them using a deep learning model, predicting future waveforms using real-time waveforms as input, predicting the posterior probability of acute deterioration triggered by changes in LV filling pressure and extracellular fluid volume, detecting abnormalities in the predicted waveforms, and triggering alerts to both patients and physicians.

Wearable electrodes, sensor systems, and devices are discreet and highly beneficial for monitoring home healthcare, allowing for freedom of movement. They are cost-effective, easy to use, compact, non-invasive, and simple yet effective. Long-term monitoring is particularly advantageous for chronic diseases like congestive heart failure (CHF). Wearable harnesses facilitate non-invasive, long-term tracking of patients. We can reap numerous benefits by monitoring and evaluating individuals diagnosed with CHF using physiological signals and derived parameters from wearable devices. These include enabling earlier interventions and diagnoses, predicting worsening heart conditions more promptly, allowing physicians to prescribe improved medications, and providing better treatment options to maximize patient outcomes. Additionally, wearable devices help avoid the necessity for expensive and invasive implantable devices, allowing users to continue their everyday activities while enjoying freedom of movement. They are comfortable to wear, easily replaceable, and cost-effective. Furthermore, these devices enable remote, long-term, continuous, and discreet monitoring of patients.

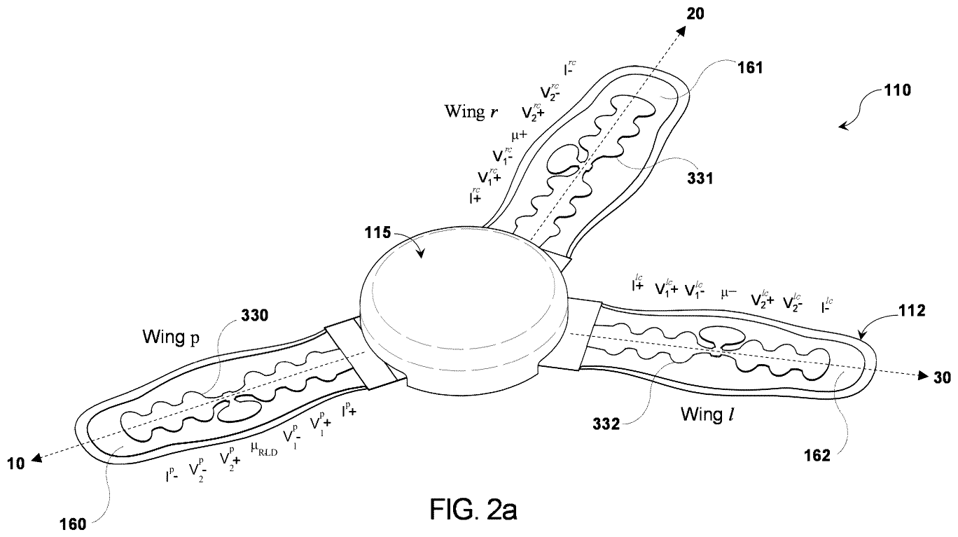

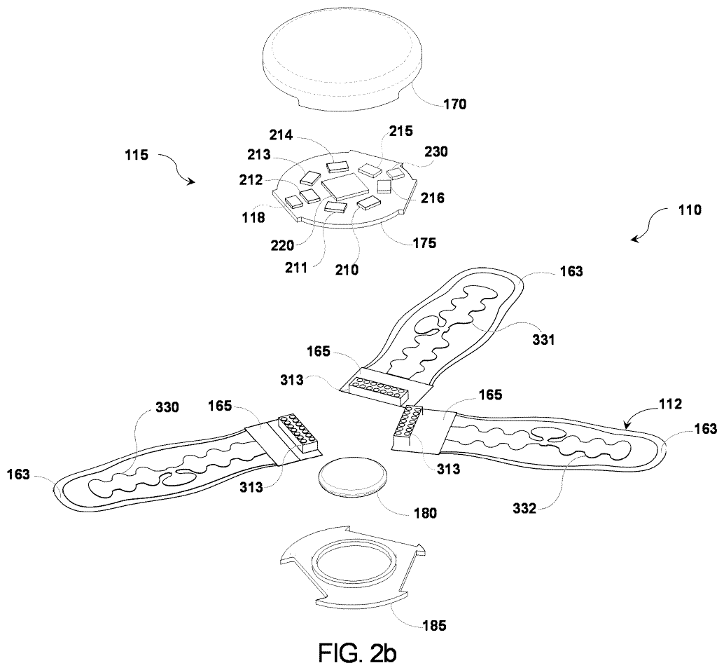

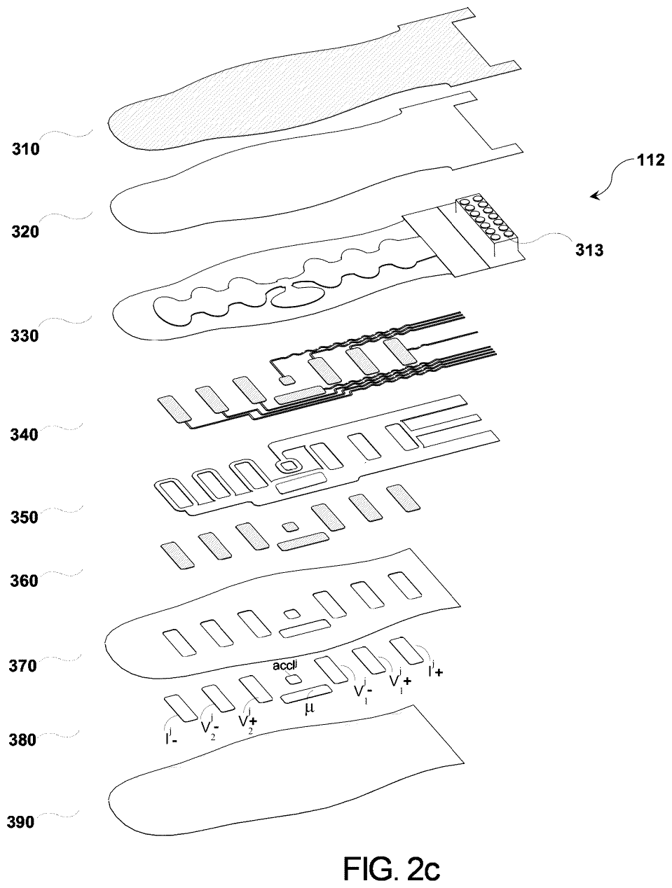

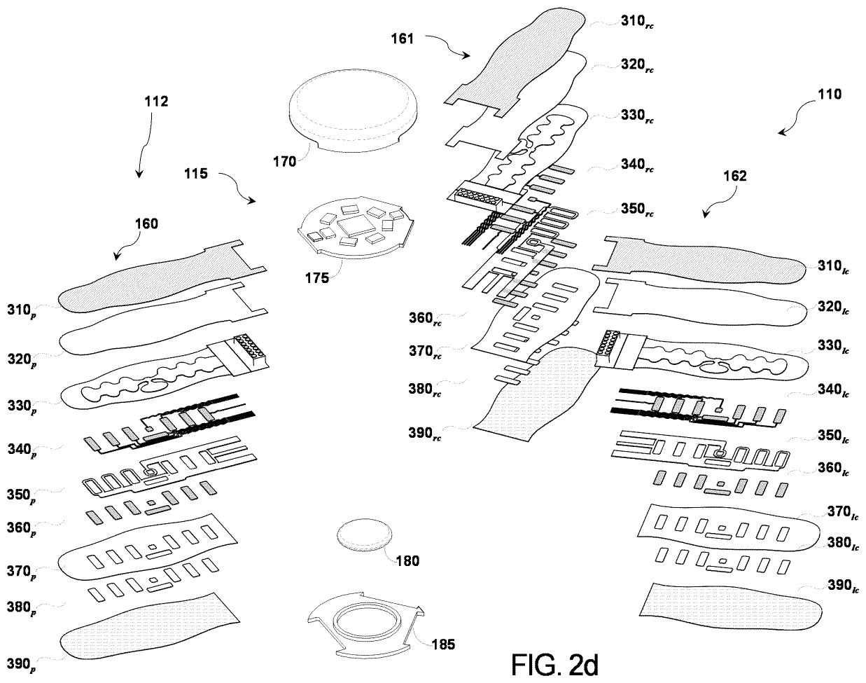

FIGS. 2a and 2b present perspective and exploded views of one embodiment of a wearable hemodynamic monitoring device 110. As illustrated in FIG. 2a, a hemodynamic monitoring device 110 features a flexible body 112 connected to a watertight and rigid housing 115. The flexible body 112, in one embodiment, includes three identical and flexible bioimpedance measuring wings 160, 161, and 162. Other configurations of the flexible bioimpedance measuring wings may also be useful. The flexible bioimpedance measuring wings may be made from ultrathin, stretchable substrate layers 330, 331, and 332 that extend laterally from the rigid housing 115. The housing 115 is constructed from various materials specifically chosen to prevent a printed circuit board (PCB) 175 with electronics inside from being deformed, especially when the user is in motion.

In contrast, the materials used for the flexible wings 160, 161, and 162 allow them to adapt to the contours of the user's body. The first bioimpedance-measuring wing 160 is positioned in the midsternal region at the upper thorax of the user. The second bioimpedance-measuring wing 161 is placed on the right side of the neck over the right common carotid artery (RCCA), while the third bioimpedance-measuring wing 162 is located on the left side of the neck over the left common carotid artery (LCCA). Each flexible wing includes multiple flexible serpentine sensors and electrode traces (not shown in FIGS. 2a and 2b) embedded within the wings and mechanically isolated from the rigid housing 115. Each electrode trace is connected to an electrode (not visible in FIG. 2a) on the bottom surface of the flexible body 112.

Similarly, each sensor trace is connected to an accelerometer sensor (also not visible in FIG. 2a) on the bottom surface of the flexible body 112. The accelerometer sensor and electrodes are designed to establish conformal contact with the user's surface, thereby facilitating the detection of acceleration, EKG, and bioimpedance waveforms. The traces allow for the transmission of electrical signals from the sensors and electrodes to the PCB assembly 175. Each wing is attached to the rigid housing 115 using a magnetic mating pad connector 313 or another suitable flexible interconnection method. Rigid housing 115 typically includes a power source, such as one or more batteries 180.

The flexible body 112 is equipped with a hinge portion 165 located at or near the junction where each wing 160, 161, and 162 meets the rigid housing 115. The hinge portions 165 are thin, flexible sections of the flexible body 112. The flexible body 112 can bend with unrestricted mobility at the point where it connects to the rigid housing 115. This feature enhances comfort by facilitating the effortless separation of the housing 115 from the user's skin during movement. The sensor and electrode traces are designed to be thin and flexible, allowing them to quickly adapt to the user's movements while ensuring that no signal distortion occurs. In various embodiments, the hinge portion 165 may be made of an ultra-thin PCB with a thickness that is preferably not more than 0.8 mm. Borders 163 designate regions of the pliable structure 112 that exhibit reduced thickness compared to the adjacent areas. The borders establish a smooth and uninterrupted connection between the flexible body 112 and the user's skin, effectively preventing any lifting of the edges and dirt infiltration underneath the flexible body 112.

Referring to FIG. 2a, the flexible wings of the monitoring device are strategically positioned to encompass the electrode locations for detecting ECG/EKG and bioimpedance waveforms. This arrangement allows for the capture of both atrial and ventricular activity. The wings also have the role of overseeing the placement of sensors to capture hemodynamic activity in the descending aorta and monitor the lung filling cycle.

In one of the embodiments, a first array of electrodes and sensors is embedded in the first bioimpedance measuring wing 160, wherein the first array includes a first (I^{p}+) and a second (I^{p}−) electrode which are positioned along the midsternal line 10 on the upper and lower aspects of the wing. The first and second electrodes are specifically designed for current injection into the upper thoracic region of the user's body. A third

$$( {V}_{1}^{p} + )$$

and a fourth

$$( {V}_{1}^{p} - )$$

electrode are positioned along the same line 10, with the third electrode located below the positive current-injecting electrode (I^{p}+) and the fourth electrode positioned below the third electrode. The third

$$( {V}_{1}^{p} + )$$

and fourth

$$( {V}_{1}^{p} - )$$

electrodes are configured for the detection of voltage at a specified location on the upper thoracic region of the user's body. Furthermore, a fifth

$$( {V}_{2}^{p} + )$$

and a sixth

$$( {V}_{2}^{p} - )$$

electrode is located along the aforementioned line 10, with the fifth electrode positioned below the fourth electrode and the sixth electrode positioned between the negative current-injecting electrode I^{p}− and the fifth electrode. The fifth

$$( {V}_{2}^{p} + )$$

and sixth

$$( {V}_{2}^{p} - )$$

electrodes are intended for the detection of voltage at a different location on the upper thoracic region of the user's body.

In one embodiment, an accelerometer sensor is placed between the fourth

$$( {V}_{1}^{p} - )$$

and fifth

$$( {V}_{2}^{p} + )$$

electrodes and for detecting motion signals along three axes. In addition, a seventh electrode (μ_{RLD}) is positioned along a line perpendicular to the midsternal line 10. The seventh electrode is located on the right side of the accelerometer sensor. The seventh electrode (μ_{RLD}), along with electrode (μ+) in wing 161 and electrode (μ−) in wing 162, is configured to detect an ECG/EKG signal from the human body.

In an embodiment, a second array of electrodes and sensors is embedded in the second bioimpedance measuring wing 161, wherein the second array includes a first (I^{rc}+) and second (I^{rc}−) electrode. The first and second electrodes of the second array are configured for an approximate location along the right common carotid artery (RCCA) 20 on the upper and lower aspects of the wing. The first and second electrode of the second array are specifically designed for current injection into the user's right neck. A third

$$( {V}_{1}^{r c} + )$$

and a fourth

$$( {V}_{1}^{r c} - )$$

electrode is configured for an approximate location along the RCCA 20, with the third electrode located below the positive current-injecting electrode I^{rc}+ and the fourth electrode positioned below the third. The third

$$( {V}_{1}^{r c} + )$$

and the fourth

$$( {V}_{1}^{r c} - )$$

electrodes are configured for the detection of voltage at a specified location on the RCCA of the user's body. Furthermore, a fifth

$$( {V}_{2}^{r c} + )$$

and a sixth

$$( {V}_{2}^{r c} - )$$

electrodes are configured for an approximate location along RCCA 20, with the fifth electrode positioned below the fourth electrode and the sixth electrode positioned between the negative current-injecting electrode I^{rc}− and the fifth electrode. The fifth

$$( {V}_{2}^{r c} + )$$

and sixth

$$( {V}_{2}^{r c} - )$$

electrodes are intended for the detection of voltage at another location on the RCCA of the user's body.

In one embodiment, an accelerometer sensor is placed between the fourth

$$( {V}_{1}^{r c} - )$$

and fifth

$$( {V}_{2}^{r c} + )$$

electrodes of the second array for detecting motion signals along three axes. In addition, a seventh electrode (μ+) is positioned along a line perpendicular to RCCA 20 on the right side of the accelerometer sensor. The seventh electrode (μ+), along with electrode (μ−) in wing 162 and electrode (μ_{RLD}) in wing 160, is configured to detect an ECG/EKG signal from the human body.

In one embodiment, a third array of electrodes and sensors is embedded in the third bioimpedance measuring wing 162, wherein the third array includes a first (I^{lc}+) and second (I^{lc}−) electrode. The first and second electrodes of the third array are configured for an approximate location along the left common carotid artery (LCCA) 30 on the upper and lower aspects of the wing. The first and second electrodes of the third array are specifically designed for current injection into the user's left neck. A third

$$( {V}_{1}^{lc} + )$$

and a fourth

$$( {V}_{1}^{lc} - )$$

electrode is configured for an approximate location along the LCCA 30, with the third electrode located below the positive current-injecting electrode I^{lc}+ and the fourth electrode positioned below the third. The third

$$( {V}_{1}^{lc} + )$$

and fourth

$$( {V}_{1}^{lc} - )$$

electrodes are configured for the detection of voltage at a specified location on the LCCA of the user's body. Furthermore, a fifth

$$( {V}_{2}^{lc} + )$$

and a sixth

$$( {V}_{2}^{lc} - )$$

electrode is configured for an approximate location along LCCA 30, with the fifth electrode positioned below the fourth electrode and the sixth electrode positioned between the negative current-injecting electrode I^{lc}− and the fifth electrode. The fifth

$$( {V}_{2}^{lc} + )$$

and sixth

$$( {V}_{2}^{lc} - )$$

electrodes are configured for the detection of voltage at another location on the LCCA of the user's body.

Furthermore, an accelerometer sensor is placed between the fourth

$$( {V}_{1}^{lc} - )$$

and fifth

$$( {V}_{2}^{lc} + )$$

electrodes and is capable of detecting motion signals along three axes. In addition, a seventh electrode (μ−) is positioned along a line perpendicular to LCCA 30 on the right side of the accelerometer sensor. The seventh electrode (μ−), along with the electrode (μ+) in wing 161 and the electrode (μ_{RLD}) in wing 160, is configured to detect an ECG/EKG signal from the human body.

FIG. 2b illustrates a partially exploded view of an embodiment of the wearable hemodynamic monitoring device 110, which offers a more detailed depiction of the individual components. As shown, the components include a rigid housing 115. In this embodiment, the rigid housing 115 includes an upper housing member 170 that can be readily connected to and disconnected from a lower housing member 185. Several components of the monitoring device 110 are situated between the upper housing member 170 and the lower housing member 185. For instance, in one embodiment, the rigid housing 115 may encompass a section of the flexible body 112, a signal acquisition unit (SAU) in the form of a PCB assembly 175, a battery 180, and three spring connectors, such as magnetic Pogo Pins (not depicted in FIG. 2b). Other types of connectors may also be useful.

Through magnetic force, the spring connectors facilitate automatic attachment and detachment from the mating pad connector 313. Pogo pin connectors offer the benefit of being exceptionally compact while withstanding up to 1 million compressions. This is attributed to their unique spring design, which maintains its dimensions and stability. The SAU 175 is placed inside housing 115 to establish contact with the sensor, electrode traces, and the battery. This contact is made possible through spring connectors on the PCB side and mating pad connectors on the traces side. Various embodiments may include or attach one or more supplementary components to the rigid housing 115.

As shown in FIG. 2b, the SAU 175 may include a plurality of analog front-end (AFE) circuits 210, 211, 212, 213, 214, 215, 216, a processor 220, a memory unit 230, a wireless transmitter module 118, and a power supply 180. In one embodiment, one of the analog front-end AFEs is used to measure 1-lead EKG/ECG waveform while a pair of AFEs is used to measure impedance plethysmography signals from each wing. For example, the electrodes μ_{RLD} in wing 160, μ+ in wing 161, and μ− in wing 162 are used to measure the electrical activities of the user's heart that are acquired by the ECG/EKG AFE 216 and digitize it to generate ECG/EKG waveform. The EKG/ECG AFE 216 may be AFE4500/4960 from Texas Instruments, USA, or AD5940/41/MAX300001 from Analog Devices, USA. Other configurations of AFEs may also be useful.

The bioimpedance measurement process in Wing 160 begins with the initialization of the SPI communication protocol. The microcontroller is configured as the SPI master, and separate Chip Select (CS) lines are assigned for AFE 210 and AFE 211. This setup allows for individual control of both Analog Front Ends (AFEs) over the SPI bus. An excitation current pulse from bioimpedance AFE 210 is administered to the user's upper thoracic region at time t=T₀ through the I^{p}+ and I^{p}− electrodes. Simultaneously, the current injection from bioimpedance AFE 211 is disabled. For example, the current injection is disabled by writing to its control register. This is accomplished by selecting AFE 211 (setting its CS line low), sending the command to turn off the current injection, and then deselecting AFE 211 (setting its CS line high). At this point, AFE 210 is configured for both current injection and voltage measurement, while AFE 211 is set up solely for voltage measurement. Consequently, AFE 210 generates the excitation current while both AFEs measure the voltage response. AFE 210 is set to output its clock to AFE 211 to synchronize the measurement cycles of the two AFEs. The SPI command of the microcontroller is configured to generate this signal, ensuring both AFEs commence their measurement cycles simultaneously. This synchronization generates a trigger signal that initiates the measurement processes, allowing AFE 210 to provide the excitation current while both AFEs capture the voltage response. This synchronized measurement yields accurate and correlated data from both AFEs. Finally, the bioimpedance measurement data is read from AFE 210 and AFE 211.

Likewise, the bioimpedance measurement process in Wing 161 begins with the initialization of the SPI communication protocol. The microcontroller is configured as the SPI master, and separate CS lines are assigned for AFE 212 and AFE 213. This setup allows for individual control of both Analog Front Ends (AFEs) over the SPI bus. An excitation current pulse from bioimpedance AFE 212 is injected into the right common carotid artery of the user at time t=T₁ through the I^{rc}+ and I^{rc}− electrodes. Simultaneously, the current injection from bioimpedance AFE 213 is disabled by writing to its control register. This is accomplished by selecting AFE 213 (setting its CS line low), sending the command to turn off the current injection, and then deselecting AFE 213 (setting its CS line high). At this point, AFE 212 is configured for both current injection and voltage measurement, while AFE 213 is set up solely for voltage measurement. Consequently, AFE 212 generates the excitation current while both AFEs measure the voltage response. AFE 212 is set to output its clock to AFE 213 to synchronize the measurement cycles of the two AFEs. The SPI command of the microcontroller is configured to generate this signal, ensuring both AFEs commence their measurement cycles simultaneously. This synchronization generates a trigger signal that initiates the measurement processes, allowing AFE 212 to provide the excitation current while both AFEs capture the voltage response. This synchronized measurement yields accurate and correlated data from both AFEs. Finally, the bioimpedance measurement data is read from AFE 212 and AFE 213.

The operating principles of AFE 212 and AFE 213 of wing 161 are mirrored for AFE 214 and AFE 215 of Wing 162, starting with the initialization of the SPI communication protocol. The microcontroller is set up as the SPI master, with distinct CS lines designated for AFE 214 and AFE 215. This configuration enables individual control of both Analog Front Ends (AFEs) via the SPI bus.

An excitation current pulse from bioimpedance AFE 214 is injected into the right common carotid artery of the user at time t=T₂ through the I^{lc}+ and I^{lc}− electrodes. At the same time, the current injection from bioimpedance AFE 215 is deactivated by writing to its control register. This process involves selecting AFE 215 (e.g., setting its CS line low), issuing the command to turn off the current injection, and then deselecting AFE 215 (e.g., setting its CS line high). At this juncture, AFE 214 is configured for both current injection and voltage measurement, while AFE 215 is arranged solely for voltage measurement. As a result, AFE 214 generates the excitation current, while both AFEs measure the voltage response. AFE 214 is also set to output its clock to AFE 215 to synchronize the measurement cycles of the two AFEs. The SPI command of the microcontroller is adapted to generate this signal, ensuring both AFEs commence their measurement cycles at the same time. This synchronization produces a trigger signal that starts the measurement processes, allowing AFE 214 to provide the excitation current while both AFEs capture the voltage response. This synchronized measurement provides accurate and correlated data from both AFEs. The bioimpedance measurement data is then retrieved from AFE 214 and AFE 215.

The bioimpedance Analog Front Ends (AFEs) 210-215 may include components such as AFE4500/4960 from Texas Instruments, USA or AD5940/41/MAX30009 from Analog Devices, USA.

The bioimpedance and EKG/ECG AFE circuits, which may include amplifiers, filters, and additional circuitry designed to convert analog bioimpedance and EKG waveforms into digital signals suitable for processing by a processor. The AFE circuits are well-established within the industry and can be sourced from a variety of reputable manufacturers, including Texas Instruments, Analog Devices, Microchip Technology, Samsung Semiconductor, Panasonic Electronics Components, STMicroelectronics, Microsemi Corporation, NXP USA Inc., among others. The SAU 175 may incorporate a storage medium that houses processor 220 and microcontroller executable code, which is responsible for capturing and converting signals from flexible electrodes and sensors into machine-readable digitized data. The processor 220 is designed to preprocess input data from the AFE circuits 210-216, the wireless transmitter module 118, and the accelerometer sensors. For instance, processor 220 may be a microcontroller unit (MCU) such as nRF5340 from Nordic Semiconductor, USA; however, other processor types may also be applicable.

In addition, the SAU 175 features a wireless module 118 that facilitates communication with smartphone devices 120 and web server 140 using Bluetooth, Wi-Fi, or alternative wireless communication standards. Such wireless modules are well-known in the industry. They are commercially available from several companies, including Texas Instruments, Microchip Technology, Samsung Semiconductor, Panasonic Electronics Components, Abracon LLC, Murata Electronics North America, and others. The preprocessed data can be transmitted to the user's mobile device for further interpretation by the wireless transmission module. The preprocessed data may also be stored on the memory unit. The memory unit 230, for example, maybe a NOR flash memory, such as an MX25R6435FZNILO-TR from Macronix. Other types of memory units may also be useful.

FIG. 2c shows another exploded view of the flexible body 112 in greater detail. For example, the exploded view illustrates a component hierarchy and multi-layered construction of the flexible body that enables the integrated chest-worn cardiovascular monitoring capabilities. The exploded view demonstrates the sophisticated layered architecture comprising both the rigid housing assembly and the flexible body construction, revealing the individual components and their spatial relationships within the overall device structure. These layers may include a top fabric layer for soft integration 310, a low-temperature hotmelt adhesive layer 320, a substrate layer 330, a series of sensor and electrode trace layers 340, 350, 360, an adhesive layer 370, an array of flexible electrodes and an accelerometer sensor 380, and a release liner 390.

In one embodiment, the uppermost layer 310 of the flexible body could be made of an elastomeric compression fabric. Other types of flexible fabrics may also be useful. The elastic fabric may be polyester with LYCRA/spandex/elastane, nylon with LYCRA/spandex/elastane, cotton-poly rib, or similar fabric. The fabric may have a variety of weaves, including plain, knit, and tricot. The elastomeric material can have a Shore 00 hardness of 30-50 and be composed of neoprene, EPDM (ethylene propylene diene monomer), polyurethanes, or other similar materials.

The low-temperature hot melt layer 320 is a thermoplastic double-sided adhesive film made of polyurethane, which is white, translucent, and has no elasticity. The melting of the low-temperature hot melt layer, for example, may be about 70° C. It has good washing resistance and is ideal for bonding and laminating various materials, including textile fabrics and polyurethane sponges. In one embodiment, the adhesive film is a hot melt adhesive web. The hot melt adhesive web is a net-like hot melt adhesive product that resembles non-woven fabrics, having characteristics of hand feel, good air permeability, and being lightweight. For example, a Thermoplastic Polyurethane (TPU) hot melt adhesive web is a thermoplastic glue that is not sticky at room temperature. However, when heated, both sides become sticky, making it useful for sticking to the top layer 310 and the substrate layer 330. It may be possible for the TPU hot melt adhesive web to stretch at the middle-temperature melting point (e.g., middle of the melting point range) but not at the low-temperature melting point (e.g., beginning of the melting point range).

In one embodiment, the top layers may include a non-woven textile material 310 coated with thermoplastic polyurethane (TPU) and a melt-processable thermoplastic elastomer. TPU is an excellent choice due to its outstanding durability, stretchability, and flexibility. Its versatile chemistry provides high tensile strength, abrasion resistance, and significant elasticity (up to 600% at break), making it a superior option to silicones like polydimethylsiloxane (PDMS). Additionally, TPU's ability to be thermoformed allows for lamination on various surfaces, ensuring long-term performance and reliability.

The top substrate layer 330 may be made of any suitable, flexible material, such as one or more flexible polymers. These polymers may include, but are not limited to, polyurethane, polyethylene, polyester, polypropylene, nylon, Teflon, and carbon-impregnated vinyl. The materials are chosen for flexibility, resilience, durability, breathability, moisture transpiration, and adhesion. To enhance moisture transpiration and breathability, substrate layers may be equipped with perforations that extend beyond the adhesive layer.

As for the sensor and electrode trace layers 340, 350, 360, materials as well as the thicknesses for forming these layers are selected for achieving the desired characteristics of flexibility, durability, signal strength, and signal transmission. One potential configuration involves utilizing multiple layers. In one embodiment, the multiple layers include conductive trace layers, such as a stretchable silver (Ag) ink layer 340 and a silver chloride (AgCl) ink layer 360, and a dielectric ink layer 350 as a separator for the conductive trace layers, such as the electrode and sensor traces. The top conductive trace 340 is in direct contact with the top substrate layer 330, while the bottom conductive trace 360 is in direct contact with the electrodes and sensor 380. The top conductive trace 340 and the bottom conductive trace 360 are interconnected through the apertures in the dielectric ink-based trace 350. In one embodiment, a stretchable silver ink can be applied as the top layer 340, a middle layer of carbon-impregnated vinyl 350, and a bottom layer of silver chloride 360. Silver has an outstanding electrical conductivity of 62.1×10⁶ Siemens/m and a very low electrical resistivity of 15.9×10⁻⁹ Ωm, which makes it an ideal choice for conductive signal traces due to their easy implementation and practical long-term usage in wearable devices.

In another embodiment, the sensor and trace layers utilize a blend of stretchable silver and carbon inks as the top conductive trace layer 340. It is imperative to note that conductive silver stretchable inks can be optimized to endure repeated stretching beyond 100% and are designed to be exceptionally durable, capable of withstanding multiple wash cycles for wearable applications. To enhance the durability and washability of a wearable hemodynamic monitor, solvent-based stretchable carbon inks are utilized over conductive silver layers. A dielectric layer is applied as a coating to protect the printed conductive traces on the PCB from environmental damage. This layer reduces silver migration under current flow or moisture conditions, thereby mitigating the risk of short circuits caused by silver ion movement between printed silver traces. Applying inert dielectric inks effectively minimizes the likelihood of silver ion migration, even when exposed to moisture. Many dielectric inks and coatings are formulated to be UV-curable due to their improved properties. UV-curable dielectrics, being entirely solid, ensure the retention of their initial form and thickness during solidification as they lack solvents or water that could evaporate during the curing process. Furthermore, these dielectrics can establish a chemical bond with the PCB surface, improving the protective barrier's resistance to specific environmental and chemical factors.

In another alternative embodiment, the top conductive trace 340 and the bottom conductive trace 360 layers may be made of silver chloride. Alternatively, each sensor and electrode trace may include two layers: a top layer of silver (Ag) and a bottom layer of silver chloride (AgCl). The choice of material for the bottom layer, such as AgCl, can be aligned with the chemistry of the hydrogel electrodes, facilitating the formation of a half-cell with the user's body. The thickness of the electrode traces can vary to optimize different characteristics. For example, at least one layer of the electrode traces should be thick enough to reduce or delay material depletion caused by the anode/cathode effect over time. Flexible electrode traces allow for better contact with the user's skin, minimizing issues like electrode peeling or detachment. This design enhances motion artifact rejection and improves signal quality by lessening stress transfer to the electrodes.

In another embodiment, both the upper layer of electrode traces 340 and the lower layer 360 may be constructed from conductive textile materials, which are separated by a dielectric layer 350. For instance, the conductive textile material could be a knitted fabric, such as the conductive Shieldex® “P130+B.” This specific fabric may be a 22% elastomer, allowing for bidirectional stretchability, with an elongation in the warp direction ranging from 155% to 205% and in the weft direction from 85% to 125%.

FIG. 2c introduces a skin adhesive layer 370 that contains holes the same size as the electrodes and sensors. Any suitable adhesive can be used to create adhesive layers. However, certain adhesives are more effective in providing long-term adhesion that is comfortable for patients' skin, causes minimal irritation, and may give a secure attachment for up to 14-30 days.

In one of the embodiments, the adhesive layer is made of stretchable, non-woven fabric coated with pressure-sensitive acrylic adhesive for prolonged skin contact. This adhesive doesn't cause irritation, toxicity, or sensitization. The advantages of pressure-sensitive acrylic adhesive include: (a) secure wear with minimal curling (14 days and longer), (b) little pain during extraction (Wong-Baker score of less than 3), (c) long-lasting wearability, (d) breathability (the MVTR of PU film is 1000 g/m²/day), (e) meets the biocompatibility criteria of ISO 10993, and (f) maintains strong adherence after a 25 kGy (gamma) exposure.

In another embodiment, ARcare® 93690, a medical-grade pressure-sensitive acrylic adhesive, is applied to a stretchable, non-woven polyurethane fabric. A clear PET release liner protects the adhesive side of this single-coated tape. This adhesive may be designed to be skin-friendly and suitable for long-term wear, making it ideal for use in wearable in-vitro diagnostic devices for periods of 14 to 21 days. The adhesive has several key characteristics: (a) it is non-toxic, non-irritating, and non-sensitizing; (b) It offers excellent adhesion and conforms well to the skin for extended wear; (c) it is breathable with a high moisture vapor transmission rate (MVTR) of greater than 675 g/m²/day, and (d) it is sterilizable up to 25 kGy. The thickness of this adhesive may be 0.008 inches. In yet another embodiment, ARcare® A-5490 is used. The adhesive is a UV curable light-yellow liquid-gel adhesive with a 1.1 g/ml density that provides water-resistant bonding and low to no residual monomer after cure. This adhesive is used to bond multiple substrate components in medical devices, including PC, PVC, ABS, PU, PS, FR4, etc. The advantages of ARcare® A-5490 adhesive include: (a) it cures effectively under UV-LED and UV-A irradiation (UVA energy >0.369 J/cm²), (b) fast curing, (c) pseudoplastic rheology designed for fluid dispensing, (d) adhesive fluoresces red under UV illumination to aid in visual inspection of the bond area, (e) it does not contain any nonreactive solvents and low outgassing when fully cured, and (f) rugged and flexible bond. ARcare® A-5490 may be designed to be skin-friendly and suitable for long-term wear, making it ideal for use in wearable in-vitro diagnostic devices for periods of up to 30 days.

Each adhesive layer 370 contains a hole designed to accommodate an electrode or an accelerometer sensor 380. The accelerometer sensor may be a 3-axis inertial measurement unit (IMU), such as a BMI270 from Bosch Sensortec, or another suitable type of 3-axis accelerometer sensor. Other types of accelerometers may also be useful. The flexible electrodes are made of materials like hydrogel to enhance the overall conformability of the flexible body. These hydrogel electrodes provide comfortable, non-irritating contact with the skin, improving electrical connection while minimizing motion artifacts. The hydrogel is highly conductive, complies with safety standards, and is certified as non-cytotoxic and non-irritating. Alternatively, the electrodes and adhesive can be replaced with a conductive layer, allowing the entire underside adhesive to function as an electrode. This hybrid adhesive/conductive layer may include substances similar to a hydrogel-silicone adhesive blend. In some embodiments, the adhesive layer may only cover a portion of the underside, leaving part of the flexible body's bottom side unadhered.

In another embodiment, electrodes 380 are made of textile as an alternative to hydrogel-based wearable monitors. The fabric material could be conductive Shieldex® “P130+B”, a two-dimensional stretchable synthetic fabric with a silver coating, a 3 mm foam layer for added comfort, and snap buttons for electrical connectivity. The composition of “P130+B” is 78% polyamide and 22% elastomer, with a coating of 99% conductive silver particles, resulting in a surface resistivity of roughly p<2 Ω/sq. In another embodiment, the electrodes could be made from carbon-enhanced adhesive electrodes.

The wearable monitor may include a protective release liner 390 to provide strength, cleanliness, and security during application. This liner can be applied over the bottom surface of the adhesive layer 370 to place the wearable device 110 on the user correctly. The liner 390 may also help protect the borders 163 and wings 160, 161, and 162 of the flexible body 112 while removing them before use. The liner can be made from materials like cardboard, thick paper, plastic, or a silicone coating on one or both sides with varying release properties to maximize effectiveness. Additionally, the liner 390 typically includes an adhesive on one side to adhere to the bottom surface of the wings on the flexible body 112.

FIG. 2d presents a fully exploded view of the hemodynamic monitoring device 110, illustrating the components. As shown, the components include the flexible body 112 and the rigid housing 115. In this embodiment, the rigid housing 115 features an upper housing member 170 that can be easily attached to or removed from a lower housing member 185. This housing may integrate a portion of the flexible body 112, a signal acquisition unit (SAU) 175, a battery 180, and three spring connectors, such as magnetic Pogo Pins (not shown).

The flexible body 112, in one embodiment, includes three identical bioimpedance-measuring wings 160, 161, and 162. The first bioimpedance wing 160 includes a top fabric layer 310ₚ, a low-temperature hot melt adhesive layer 320ₚ, a substrate layer 330ₚ, a series of sensor and electrode trace layers 340ₚ, 350ₚ, 360ₚ, an adhesive layer 370ₚ, an array of flexible electrodes and an accelerometer sensor 380ₚ, along with a liner 390ₚ. Similarly, the second bioimpedance wing 161 includes a top fabric layer 310_{rc}, a low-temperature hot melt adhesive layer 320_{rc}, a substrate layer 330_{rc}, a series of sensor and electrode trace layers 340_{rc}, 350_{rc}, 360_{rc}, an adhesive layer 370_{rc}, an array of flexible electrodes and an accelerometer sensor 380_{rc}, and a liner 390_{rc}. The third bioimpedance wing 162 follows the same structure with its respective top fabric layer 310_{lc}, low-temperature hot melt adhesive layer 320_{lc}, substrate layer 3301, sensor and electrode trace layers 340_{lc}, 350_{lc}, 360_{lc}, adhesive layer 370_{lc}, an array of flexible electrodes and accelerometer sensor 380_{lc}, and liner 390I_{c}.

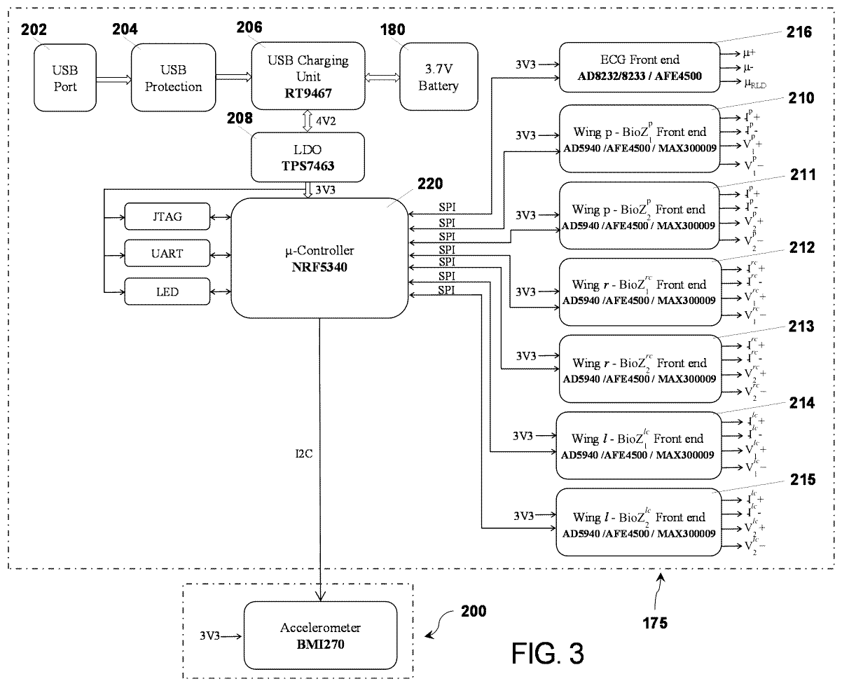

FIG. 3 illustrates an embodiment of a PCB corresponding to a signal acquisition unit (SAU) 175 and wing x PCB 200 (x=160, 161, and 162). The SAU-PCB includes various digital circuits or sub-systems for performing various functions of the wearable device. The SAU is designed to be suitable for integration into the hemodynamic monitoring device 110. Although this is preferred, the SAU could also be located separately.

Referring to FIG. 3, one embodiment of the SAU-PCB includes a USB port 202, a USB protection module 204, a USB power module 206, a battery module 180, an LDO 208, various analog front-end circuits for receiving data from the sensors and electrodes of the wearable device 210-216 and a microcontroller 220. The USB port provides a USB connection to the SAU-PCB. As for the USB protection module, it protects the SAU-PCB from power surges from connections to the USB port. The USB power module enables charging of the battery in the battery module through the USB port. The battery, for example, may be a 3.7V battery (1 amp). Other types of batteries may also be helpful. In addition, the USB power module includes a power switch for switching the system on and off. In one embodiment, a power converter module converts input voltage from either the USB port or directly from the rechargeable battery to voltages required to power the different subsystems of the entire circuit system. For example, the power converter regulates the power to the various components of the PCBs. The power converter module, for example, may include an LDO, such as TPS746 from Texas Instruments, USA. Other types of power converters may also be helpful.

The Analog front-end circuits for bioimpedances 210, 211, 212, 213, 214, 215 can have multiple modulation and demodulation circuits to measure bioimpedances at various locations in the upper thoracic region as well as in the neck regions around the right and left carotid arteries. An Analog front-end circuit for EKG/ECG 216 can have various amplifier and filter circuits to measure multiple ECG signals. The front-end circuit for bio-impedances 210-215, the front-end circuit for EKG 216, and the front-end circuit for 3-axis accelerometer 200 are connected to the processing unit 220, e.g., a microprocessor, to allow the processing of the signals. The front-end circuit for the accelerometer detects a patient's posture and activity. This front-end includes amplifiers and filters to process the signals from the accelerometer center. The processed signals are used to detect patients' posture and activity. The accelerometer sensor may also compensate for the signals affected by the patient's movements. A power supply 180 provides the proper voltages and power to each circuit from a battery to power up the SAU 175.

Such Analog Front End circuits, which include amplifiers, filters, and associated circuitry for converting analog sensor signals into digital signals that can processed by a microprocessor, are well known in the art and are commercially available from a variety of sources, including Texas Instruments, Microchip Technology, Samsung Semiconductor, Panasonic Electronics Components, STMicroelectronics, Microsemi Corporation, NXP USA Inc, Analog Devices, etc.

The SAU 175 also includes a memory or local storage medium for storing the code or software for operating the microprocessor 220 and storing data, including data received from the sensors. In a particular embodiment, the microprocessor 220 may have an in-built memory such as nRF5340 from Nordic Semiconductor, USA.

The SAU 175 is equipped with a wireless module that facilitates wireless communication with the smartphone 120 and web server 140 through Bluetooth, Wi-Fi, or other recognized wireless communication standards. Such wireless modules are widely recognized in the industry and are commercially available from numerous suppliers, including Texas Instruments, Microchip Technology, Samsung Semiconductor, Panasonic Electronics Components, Abracon LLC, and Murata Electronics North America, among others. In a specific embodiment, the microprocessor 220 may incorporate an integrated memory such as the nRF5340 provided by Nordic Semiconductor, USA.

In an embodiment, the microcontroller 220 has an in-built storage medium that contains a microprocessor or microcontroller executable code that captures and converts the signals from the electrodes and accelerometer sensors into machine-readable digitized data. The code also creates arrays of digitized data stored in a traditional file system for subsequent retrieval in the local storage medium. This storage medium is a non-volatile memory that can be erased and programmed. The code also transmits and receives data and commands to and from an internet-connected database service that resides in a remote physical database server such as a web server 140 and to and from a smartphone 120 through a wireless module 118. The code can communicate with the server directly and transfer the acquired data for a patient if a smartphone 120 is not within the wireless communication range of the signal acquisition unit 175.

Before applying the hemodynamic monitoring device 110 on a user 105, the patient's skin is typically prepared by shaving a small portion around the midsternal region and then rubbing and cleaning the area. Subsequently, adhesive covers are removed from the adhesive layers 370ₚ, 370_{rc}, and 370_{lc} for the wings 160, 161, and 162, thereby exposing the adhesive layers. Concurrently, the liners 310ₚ, 310_{rc} and 310_{lc} serve as a support for the flexible body 112, offering a grip for the physician or operator and preventing the flexible body 112 and its borders 163 from folding or forming wrinkles during the removal of adhesive covers. Liners 310ₚ, 310_{rc}, and 310_{lc} can be constructed from a relatively stiff, firm material to support the flexible body 112 while applying device 310ₚ, 310_{rc}, and 310_{lc} to the skin.

Once the device 110 is applied onto the skin, pressure can be exerted on the flexible body 112 to press it onto the chest and the right and left neck to help ensure that the device 110 adheres to the skin. Then, the liners 310ₚ, 310_{rc}, and 310_{lc} are removed from the flexible body 112. After removing the liners, pressure may be reapplied to the flexible body 112 to ensure proper adherence to the skin. Lastly, the upper housing member 170 should be pressed to switch on the hemodynamic monitoring device 110.

Signal processing unit 175 is programmed to activate three different patch wings, 160, 161, and 162, in sequence. This facilitates remote monitoring of deteriorating hemodynamic parameters in heart failure patients.

When a signal acquisition unit 175 and a smartphone 120 are powered on, the signal acquisition unit and the smartphone are connected wirelessly. A monitoring App on the smartphone may serve as an interface to perform the setup of as well as operate the monitoring device. In one embodiment, wing 160 is activated and configured to measure hemodynamic parameters such as ECF, stroke volume, cardiac output, heart rate, and heart rate variability from the upper thoracic region. Based on the guidelines provided to patients by their treating clinicians, this operation can be set for a minimum of one minute to a maximum of several minutes. Following the operation of wing 160, wing 161 at the right carotid artery gets activated and configured to measure hemodynamic parameters such as LV end-diastolic pressure (LVEDP). Wing 161's operation can be set for a minimum of one minute to a maximum of several minutes, depending on the guidelines provided to patients by their treating clinicians. Following the operation of wing 161, wing 162 at the left carotid artery is activated and configured to measure other relevant hemodynamic parameters. Depending on the guidelines provided to patients by the clinicians, wing 162's operation can be set for a minimum of one minute to a maximum of several minutes.

The smartphone controls the signal acquisition unit by sending commands to the signal acquisition unit wirelessly, such as Bluetooth, Wi-Fi, or other wireless communication standards. Commands from the smartphone to the acquisition unit include initializing the acquisition unit, requesting to send data to the smartphone for signal quality check at the beginning stage of the test, requesting start and stop the test, and requesting to upload stored data in the storage of the acquisition unit wirelessly after the test is completed. In addition to communicating with the acquisition unit, the smartphone 120 sends automatically logged symptom data—triggered when clinical parameters measured by the wearable monitoring device exceed baseline thresholds to the portal or server. Once the test is started, the signal acquisition unit acquires and processes sensor signals. The processed signals are stored in the built-in flash memory of the processor 220. Once the test is completed, the stored data is transferred and uploaded to the web-based server 140 through a wireless connection.

After the wearable monitoring device 110 is switched on, real-time EKG/ECG, bioimpedance waveforms

$${BioZ}_{1}^{j} = ( {V}_{1}^{j} + - {V}_{1}^{j} - ) / ( {I}^{j} + - {I}^{j} - ) and {BioZ}_{2}^{j} = ( {V}_{2}^{j} + - {V}_{2}^{j} - ) / ( {I}^{j} + - {I}^{j} - ) ,$$

and 3-axis motion signal data are collected for each wing (j=p, r, l). Once the real-time waveforms are sent to a web-based server 140 via an IoT gateway, a deep-learning model is enabled to predict these waveforms several days to months in advance. Subsequently, the predicted waveforms facilitate the measurement of pulse transit time (PTT) at the pulmonary region, as well as the right and left carotid arteries. The PTT at the pulmonary region is required to measure PTT-corrected pulse arrival time i.e.,

$${PEP}_{c}^{p} .$$

Similarly, the PTT at the right and left carotid arteries is essential for assessing right and left carotid pressure and providing a method to obtain left ventricular end-diastolic pressure (LVEDP). Through diligent monitoring of these parameters, potential heart failure events can be detected early, thereby preempting the onset of pulmonary edema and reducing hospitalization frequency.

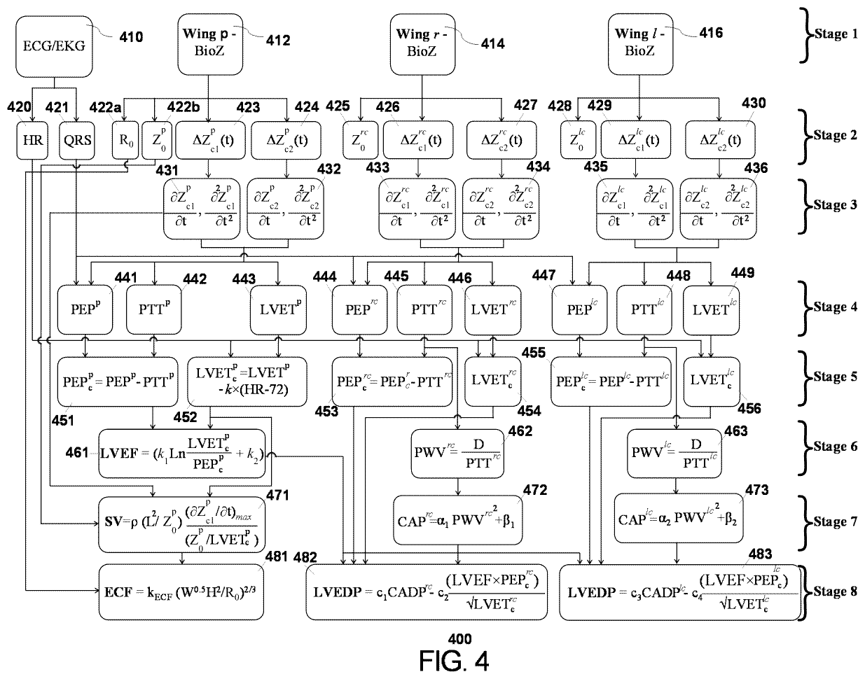

FIG. 4 shows a software flow 400 for extracting clinical parameters from data measured from the sensors and electrodes of the wearable device. The wearable device is similar to that described in FIGS. 2a-2d. In one embodiment, clinical parameter extraction is performed by a cloud server. For example, preprocessed data from the wearable device is transmitted to the user's mobile device and then transmitted to the server. Alternatively, the preprocessed data from the wearable device may be directly transmitted to the server. For example, the main PCB may include a mobile communication module, such as a Wi-Fi or BLE module, enabling the preprocessed data to be transmitted to the cloud server via a cellular network. The flow, for example, may be performed by a software application running on the server.

The flow, as shown, includes multiple stages for processing the data collected from the wearable device. The flow, in one embodiment, includes eight stages (stages 1 to 8). Providing a flow with other numbers of stages may also be helpful.

At stage 1, physiological data collected from the wearable device is received. For example, stage 1 may be referred to as an input stage. In one embodiment, EKG/ECG PS waveform from μ+, μ− and μ_{RLD} electrodes are received at 410,

$${BioZ}_{1}^{p} ( {V}_{1}^{p} + , {V}_{1}^{p} - ) and {BioZ}_{2}^{p} ( {V}_{2}^{p} + , {V}_{2}^{p} - )$$

data from the I^{p}+ and I^{p}− electrodes of wing p is received at 412,

$${BioZ}_{1}^{rc} ( {V}_{1}^{rc} + , {V}_{1}^{rc} - ) and {BioZ}_{2}^{rc} ( {V}_{2}^{rc} + , {V}_{2}^{rc} - )$$

data from the I^{rc}+ and I^{rc}− electrodes of wing r is received at 414, and

$${BioZ}_{1}^{lc} ( {V}_{1}^{lc} + , {V}_{1}^{lc} - ) and {BioZ}_{2}^{lc} ( {V}_{2}^{lc} + , {V}_{2}^{lc} - )$$

data from the I^{lc}+ and I^{lc}− electrodes of wing l isreceived at 416.

In an embodiment, the flow employs the input data at 410 to automatically measure QRS duration (QRS_{d}), heart rate (HR) and heart rate variability (HRV) from EKG/ECG waveforms.

The flow employs the input data at 412 to automatically measure

$${BioZ}_{1}^{p} and {BioZ}_{2}^{p}$$

waveforms

$$( {BioZ}_{1}^{p} = {Z}_{0}^{p} + Δ {Z}_{c 1}^{p} + Δ {Z}_{r 1}^{p} , {BioZ}_{2}^{p} = {Z}_{0}^{p} + Δ {Z}_{c 2}^{p} + Δ {Z}_{r 2}^{p} )$$

from wing p.

$${BioZ}_{1}^{p} and {BioZ}_{2}^{p}$$

may be measured around a frequency of f=50-100 kHz by applying an excitation current ˜100 μA-400 μA between I^{p}+ and I^{p}− electrodes on wing 160. The base impedance

$${Z}_{0}^{p}$$

corresponds to non-time varying tissues, such as muscle, bone and fat.

$$Δ {Z}_{c 1}^{p} and Δ {Z}_{r 1}^{p}$$

correspond to the cardiac and respiratory components of

$${BioZ}_{1}^{p} , Δ {Z}_{c 2}^{p} and Δ {Z}_{r 2}^{p}$$

correspond to the cardiac and respiratory components of

$${BioZ}_{2}^{p} \cdot {Z}_{0}^{p}$$

is measured when the pulsatile volume is at a minimum. The respiratory components

$$Δ {Z}_{r 1}^{p} and Δ {Z}_{r 2}^{p}$$

are obtained by filtering

$${BioZ}_{1}^{p} and {BioZ}_{2}^{p}$$

signals using first a Low Pass FIR (LPF) filter at 10 Hz to eliminate the high frequency noise, and after a low pass interpolated FIR filter at 0.5 Hz. The cardiac components

$$Δ {Z}_{c 1}^{p} and Δ {Z}_{c 2}^{p}$$

are obtained by first applying a linear detrend and zero-mean function to the

$${BioZ}_{1}^{p} and {BioZ}_{2}^{p}$$

signal respectively, and then filtering out those signals using a band pass FIR filter with cut-off frequencies at 0.9 Hz and 7 Hz, respectively. The cut-off frequencies of the band pass FIR filter may be changed, depending on the results obtained using the bioimpedance AFE. After the respiratory components are suppressed, the signal

$${BioZ}_{1}^{p} ( t )$$

will be equivalent to the parallel of the DC component

$${Z}_{0}^{p}$$

and the dynamic AC component

$$Δ {Z}_{c 1}^{p} ( t ) ,$$

also known as cardiogenically induced pulsatile impedance change. The new thoracic impedance signal is described as:

$${BioZ}_{1}^{p} ( t ) = {Z}_{0}^{p} ‖ Δ {Z}_{c 1}^{p} ( t ) , and {BioZ}_{2}^{p} ( t ) = {Z}_{0}^{p} ‖ Δ {Z}_{c 2}^{p} ( t ) .$$

In an embodiment, the flow employs the input data at 414 to automatically measure

$${BioZ}_{1}^{rc} and {BioZ}_{2}^{rc}$$

waveforms

$$( {BioZ}_{1}^{rc} = {Z}_{0}^{rc} + Δ {Z}_{c 1}^{rc} + Δ {Z}_{r 1}^{rc} , {BioZ}_{2}^{rc} = {Z}_{0}^{rc} + Δ {Z}_{c 2}^{rc} + Δ {Z}_{r 2}^{rc} )$$

from wing r.

$${BioZ}_{1}^{rc} and {BioZ}_{2}^{rc}$$

may be measured around a frequency of f=100 kHz, by applying an excitation current ˜100 μA between I^{rc}+ and I^{rc}− electrodes on wing 161. The base impedance

$${Z}_{0}^{rc}$$

corresponds to non-time varying tissues, such as muscle, bone and fat.

$$Δ {Z}_{c 1}^{rc} and Δ {Z}_{r 1}^{rc}$$

correspond to the cardiac and respiratory components of

$${BioZ}_{1}^{rc} and Δ {Z}_{c 2}^{rc} and Δ {Z}_{r 2}^{rc}$$

correspond
to the cardiac and respiratory components of

$${BioZ}_{2}^{rc} \cdot {Z}_{0}^{rc}$$

is measured when the pulsatile volume is at a minimum. The respiratory components

$$Δ {Z}_{r 1}^{rc} and Δ {Z}_{r 2}^{rc}$$

are obtained by filtering

$${BioZ}_{1}^{rc} and {BioZ}_{2}^{rc}$$

signals using first a Low Pass FIR (LPF) Filter at 10 Hz to eliminate the high-frequency noise and after a low pass interpolated FIR filter at 0.5 Hz. The cardiac components,

$$Δ {Z}_{c 1}^{rc} and Δ {Z}_{c 2}^{rc}$$

are obtained by first applying a linear detrend and zero-mean function to the

$${BioZ}_{1}^{rc} and {BioZ}_{2}^{rc}$$

signals, respectively, and then those signals are filtered using a band pass FIR filter with cut-off frequencies at 0.9 Hz and 7 Hz, respectively. The cut-off frequencies of the band pass FIR filter may be changed, depending on the results obtained using bioimpedance AFE. After the respiratory components are suppressed, the signal

$${BioZ}_{1}^{rc} ( t )$$

will be equivalent to the parallel of the DC component

$${Z}_{0}^{rc}$$

and the dynamic AC component

$$Δ {Z}_{c 1}^{rc} ( t ) ,$$

also known as cardiogenically induced pulsatile impedance change. The right neck bioimpedance signal is described as:

$${BioZ}_{1}^{rc} ( t ) = {Z}_{0}^{rc} ‖ Δ {Z}_{c 1}^{rc} ( t ) , and {BioZ}_{2}^{rc} ( t ) = {Z}_{0}^{rc} ‖ Δ {Z}_{c 2}^{rc} ( t ) .$$

In an embodiment, The flow employs the input data at 416 to automatically measure the

$${BioZ}_{1}^{lc} and {BioZ}_{2}^{lc}$$

waveforms

$$( {BioZ}_{1}^{lc} = {Z}_{0}^{lc} + Δ {Z}_{c 1}^{lc} + Δ {Z}_{r 1}^{lc} , {BioZ}_{2}^{lc} = {Z}_{0}^{lc} + Δ {Z}_{c 2}^{lc} + Δ {Z}_{r 2}^{lc} )$$

from wing l.

$${BioZ}_{1}^{lc} and {BioZ}_{2}^{lc}$$

may be measured around a frequency of f=100 kHz, by applying an excitation current ˜100 μA between I^{lc}+ and I^{lc}− electrodes on wing 162. The base impedance

$${Z}_{0}^{lc}$$

corresponds to non-time varying tissues, such as muscle, bone and fat.

$$Δ {Z}_{c 1}^{lc} and Δ {Z}_{r 1}^{lc}$$

correspond to the cardiac and respiratory components of

$${BioZ}_{1}^{lc} , and Δ {Z}_{c 1}^{lc} and Δ {Z}_{r 2}^{lc}$$

correspond to the cardiac and respiratory components of

$${BioZ}_{2}^{lc} \cdot {Z}_{0}^{lc}$$

is measured when the pulsatile volume is at a minimum. The respiratory components

$$Δ {Z}_{r 1}^{lc} and Δ {Z}_{r 2}^{lc}$$

are obtained by filtering

$${BioZ}_{1}^{lc} and {BioZ}_{2}^{lc}$$

signals using first a Low Pass FIR (LPF) Filter at 10 Hz to eliminate the high frequency noise, and after a low pass has interpolated FIR filter at 0.5 Hz. The cardiac components,

$$Δ {Z}_{c 1}^{lc} and Δ {Z}_{c 2}^{lc}$$

are obtained by first applying a linear detrend and zero-mean function to the

$$B i o {Z}_{1}^{lc} and {BioZ}_{2}^{lc}$$

signals, respectively, and then those signals are filtered using a band pass FIR filter with cut-off frequencies at 0.9 Hz and 7 Hz, respectively. The cut-off frequencies of the band pass FIR filter may be changed, depending on the results obtained using bioimpedance AFE. After the respiratory components are suppressed, the signal

$$B i o {Z}_{1}^{lc} ( t )$$

will be equivalent to the parallel of the DC component

$${Z}_{0}^{lc}$$

and the dynamic AC component

$$Δ {Z}_{c 1}^{lc} ( t ) ,$$

also known as cardiogenically induced pulsatile impedance change. The left neck bioimpedance signal is described as:

$$B i o {Z}_{1}^{lc} ( t ) = {Z}_{0}^{lc} ‖ Δ {Z}_{c 1}^{lc} ( t ) , and {BioZ}_{2}^{lc} ( t ) = {Z}_{0}^{lc} ‖ Δ {Z}_{c 2}^{lc} ( t ) .$$

At stage 2, the input data is used to calculate heart rate (HR) at 420, QRS duration (QRS_{d}) at 421, R₀ at 422a,

$${Z}_{0}^{p} at 422 b , Δ {Z}_{c 1}^{p} ( t ) at 423 , Δ {Z}_{c 2}^{p} ( t ) at 424 , {Z}_{0}^{r c} at 425 , Δ {Z}_{c 1}^{r c} ( t ) at 426 , {ΔZ}_{c 2}^{r c} ( t ) at 427 , {Z}_{0}^{{lc}_{}} at 428 , Δ {Z}_{c 1}^{l c} at 429 and {ΔZ}_{c 2}^{lc} at 430.$$

Other parameters may also be determined at stage 2. For example, although it is not illustrated, input data may be employed to determine different parameters, such as respiratory rate (RR).

In one embodiment, the Cole-Cole impedance parameter R₀ is calculated at 422a using a time-domain fitting approach based on single-frequency DC-biased sinusoidal excitation, and was utilized to measure extracellular fluid (ECF) via a wearable patch configured for application on wing 160.

The algorithm for calculating R₀ is implemented through a sequence of steps involving initial estimations followed by iterative fitting processes, wherein each step is configured to refine the accuracy of the calculation based on predetermined mathematical models and formulas, with execution performed in accordance with algorithmic logic embedded in the processing module.
Input: The algorithm for estimating the Cole impedance parameter is executed using a sequential method wherein two input signals—namely, the excitation current signal u(t) and the output response signal y(t) were processed over the time interval, where t belongs to the interval [0, T+TJ]. The excitation signal u(t) is defined by a superposition of a sinusoidal component and a DC-bias term, mathematically expressed as:

$$u : = {I}_{0} cos ( 2 π n f / {f}_{s} + ϕ ) + {I}_{1} . \qquad ( 1 )$$

where
   I₀ represents the sinusoidal current amplitude (ranging from 80 to 100 μA),
   f is the excitation frequency ˜50-100 kHz, φ is the initial phase, I₁ corresponds to the DC bias amplitude,
   fₛ is the sampling frequency, N is the number of sampling points, and
   n∈[1, 2, 3, . . . , N].
The output signal y(t) was processed in conjunction with the Cole impedance model, which is expressed using a fractional-order matrix framework referred to as the fractional operating matrix (FOM). The impedance Z(F) was defined as a normalized N×N matrix,

$$Z ( {F}_{α} ) : = \frac{a {F}_{α} + b A}{{F}_{α} + c A} \qquad ( 2 )$$

$$where$$

$$a := {R}_{0} ,$$

$$b := {R}_{∞} {τ}_{0}^{α} ,$$

$$c := {τ}_{0}^{α} ,$$

A is an identity matrix of size N×N, and
F_{α} is defined as

$${F}_{α} : = {( \frac{T}{N} )}^{α} \frac{1}{Γ ( α + 2 )} [ \begin{matrix}1 & {ξ}_{2} & {ξ}_{3} & … & {ξ}_{N - 1} & {ξ}_{N} \\ 0 & 1 & {ξ}_{2} & {ξ}_{3} & … & {ξ}_{N - 1} \\ 0 & 0 & 1 & {ξ}_{2} & … & {ξ}_{N - 2} \\ 0 & 0 & 0 & 1 & ⋱ & ⋮ \\ ⋮ & ⋮ & ⋮ & ⋮ & ⋱ & {ξ}_{2} \\ 0 & 0 & 0 & 0 & … & 1\end{matrix} ] \qquad ( 3 )$$

with
   ξₚ=p^{α+1}−2 (p−1)^{α+1}+(p−2)^{α+1} for p=2, 3, . . . , N, and
   Γ(⋅) is the Gamma function.
These matrix structures were utilized in computing the voltage response through the expression:

$$\tilde{y} = u Z ( {F}_{α} ) = u \frac{a {F}_{α} + b A}{{F}_{α} + c A} \qquad ( 4 )$$

The algorithm to estimate the Cole-Cole impedance parameter is obtained using the following method:
Step 1: Calculation—The parameter estimation process is initiated by identifying the steady-state interval Tₖ corresponding to k excitation periods.
Within this interval, estimates of R₀ and R_{∞} are derived from integrals involving the response signal y(t) and components of the excitation signal u(t), weighted by sine and cosine basis functions. Intermediate quantities such as I₁, Qᵤ, I₀, and Q_{y} are computed to facilitate the numerical isolation of resistive (R) and reactive (X) components using predetermined relationships.

$${I}_{1} ← \frac{1}{Δ {T}_{k}} {\int }^{Δ {T}_{k}} u ( t ) dt \qquad ( 5 a )$$

$${I}_{u} ← {\int }^{Δ {T}_{k}} u ( t ) cos ( ω t ) dt \qquad ( 5 b )$$

$${Q}_{u} ← {\int }^{Δ {T}_{k}} u ( t ) sin ( ω t ) dt \qquad ( 5 c )$$

$${I}_{0} ← \frac{1}{Δ {T}_{k}} \sqrt{{I}_{u}^{2} + {Q}_{u}^{2}} \qquad ( 5 d )$$

$$y ( t ) {\vert}_{t = {0}^{+}} ← {lim}_{t \to 0} y ( t ) \qquad ( 5 e )$$

$${I}_{Y} ← {\int }^{Δ {T}_{k}} y ( t ) cos ( ω t ) dt \qquad ( 5 f )$$

$${Q}_{y} ← {\int }^{Δ {T}_{k}} y ( t ) sin ( ω t ) dt \qquad ( 5 g )$$

The parameters R and X are calculated as follows:

$$R ← \frac{( {I}_{y} {I}_{u} + {Q}_{y} {Q}_{u} )}{( {I}_{u}^{2} + {Q}_{u}^{2} )} \qquad ( 6 a )$$

$$X ← \frac{( {I}_{y} {Q}_{u} + {Q}_{y} {I}_{u} )}{( {I}_{u}^{2} + {Q}_{u}^{2} )} \qquad ( 6 b )$$

Step 2: Initial Estimation of R₀ and R_{∞}:
   R_{∞} is estimated using the formula:

$${R}_{∞}^{[ 0 ]} ← \frac{y ( t ) {\vert}_{t = 0 +}}{{I}_{0} cos ( ϕ ) + I 1} \qquad ( 7 a )$$

R₀ is estimated as:

$${R}_{0}^{[ 0 ]} ← \frac{1}{Δ {T}_{k} {I}_{1}} {\int }^{Δ {T}_{k}} y ( t ) dt \qquad ( 7 b )$$

Subsequently, the dispersion parameter α was calculated using a normalized phase approximation, beginning with an initial estimate derived from (2/π) and refined iteratively through formula (10j).
Step 3: Initial Estimation of α:
   The parameter α is estimated using the following formulas:

$$A ← \frac{( {R}_{0 -} {R}_{∞} ) ( R - {R}_{∞} )}{{( R - {R}_{∞} )}^{2} + {X}^{2}} \qquad ( 8 a )$$

$$B ← - \frac{( {R}_{0 -} {R}_{∞} ) X}{{( R - {R}_{∞} )}^{2} + {X}^{2}} \qquad ( 8 b )$$

The initial value of α is then calculated as:

$${α}^{[ 0 ]} = ( 2 / π ) {tan}^{- 1} \frac{B}{A - 1} \qquad ( 8 c )$$

Step 4: Initial Estimation of τ₀:
   The relaxation time constant τ₀ is estimated using:

$${τ}_{0}^{[ 0 ]} = \frac{1}{w} [ {( A - 1 )}^{2} + {B}^{2} ] \frac{1}{2 {α}^{[ 0 ]}} \qquad ( 9 )$$

Step 5: Iterative Complex Non-Linear Least Squares (CNLS) Fitting:
   Initial values of the parameters a, b, c, τ₀, and α are obtained using:

$${a}^{[ 0 ]} ← {R}_{0}^{[ 0 ]} \qquad ( 10 a )$$

$${b}^{[ 0 ]} ← {{R}_{∞}^{[ 0 ]} ( {τ}_{0}^{[ 0 ]} )}^{{α}^{[ 0 ]}} \qquad ( 10 b )$$

$${c}^{[ 0 ]} ← {( {τ}_{0}^{[ 0 ]} )}^{{α}^{[ 0 ]}} \qquad ( 10 c )$$

$${θ}^{[ 0 ]} ← \{ {a}^{[ 0 ]} , {b}^{[ 0 ]} , {c}^{[ 0 ]} , {α}^{[ 0 ]} \} and m ← 1 \qquad ( 10 d )$$

$$- {Z}^{[ m ]} ( {F}_{α} ) = \frac{{a}^{[ m - 1 ]} {F}_{α}^{[ m - 1 ]} + {b}^{[ m - 1 ]} A}{{F}_{α}^{[ m - 1 ]} + {c}^{[ m - 1 ]} A} \qquad ( 10 e )$$

$${min}_{θ} {‖ y - {uZ}^{[ m ]} ( {F}_{α} ) ‖}_{2}^{2} \qquad ( 10 f )$$

$$m ← m + 1 \qquad ( 10 g )$$

$$θ [ m - 1 ] ← \hat{θ} \qquad ( 10 h )$$

$$\{ \hat{a} , \hat{b} , \hat{c} , \hat{α} \} ← \hat{θ} \qquad ( 10 i )$$

$$← \hat{a} , ← \hat{\frac{b}{\hat{c}}} , ← {\hat{c}}^{1 / \hat{α}} \qquad ( 10 j )$$

All algorithmic steps are structured to enhance estimation accuracy through recursive fitting logic, with the complete process executed within a digital processing module configured to perform time-domain computations under embedded firmware control.

At stage 3, the flow computes

$$\partial {Z}_{c 1}^{p} / \partial t$$

(i.e., first derivative of

$${Z}_{c 1}^{p} ( t )$$

with time t) and

$${\partial }^{2} {Z}_{c 1}^{p} / \partial {t}^{2}$$

(i.e., 2^{nd} derivative of

$${Z}_{c 1}^{p} ( t )$$

with time t) at 431,

$$\partial {Z}_{c 2}^{p} / \partial t$$

(i.e., first derivative of

$${Z}_{c 2}^{p} ( t )$$

with time t) and

$${\partial }^{2} {Z}_{c 2}^{p} / \partial {t}^{2}$$

(i.e., 2^{nd} derivative of

$${Z}_{c 2}^{p} ( t )$$

with time t) at 432; however, at different times t=T₀ and at t=T₁, respectively. The flow also computes

$$\partial {Z}_{c 1}^{r c} / \partial t$$

(i.e., first derivative of

$${Z}_{c 1}^{r c} ( t )$$

with time t) and

$${\partial }^{2} {Z}_{c 1}^{r c} / \partial {t}^{2}$$

(i.e., 2^{nd} derivative of

$${Z}_{c 1}^{r c} ( t )$$

with time t) at 433,

$$\partial {Z}_{c 2}^{r c} ( t ) / \partial t$$

(i.e., first derivative of

$${Z}_{c 2}^{r c} ( t )$$

with time t) and

$${\partial }^{2} {Z}_{c 2}^{r c} / \partial {t}^{2}$$

(i.e., 2^{nd} derivative of

$${Z}_{c 2}^{r c} ( t )$$

with time t) at 434 at different times t=T₂ and at t=T₃, respectively. The flow further computes

$$\partial {Z}_{c 1}^{lc} / \partial t$$

(i.e., first derivative of

$${Z}_{c 1}^{lc} ( t )$$

with time t) and

$${\partial }^{2} {Z}_{c 1}^{lc} / \partial {t}^{2}$$

(i.e., 2^{nd} derivative of

$${Z}_{c 1}^{lc} ( t )$$

with time t) at 435,

$$\partial {Z}_{c 2}^{lc} / \partial t$$

(i.e., first derivative of

$${Z}_{c 2}^{l c} ( t )$$

with time t) and

$${\partial }^{2} {Z}_{c 2}^{lc} / \partial {t}^{2}$$

(i.e., 2^{nd} derivative of

$${Z}_{c 2}^{lc} ( t )$$

with time t) at 436 at different times t=T₄ and at t=T₅, respectively.

At stage 4, the flow assesses the systolic time intervals at each wing, including the pre-ejection period (PEP) and left ventricular ejection time (LVET). PEP is determined from the EKG and BioZ waveforms by measuring the time interval between the peak of the Q-wave in the QRS complex and the B point on the first derivative of the BioZ waveforms. In one embodiment, the PEP^{p} at 441 represents extended or delayed PEP based on the time from the onset of QRS or ventricular pacing pulse to the onset of central aortic flow. Similarly, the PEP^{rc} at 444 represents extended or delayed PEP based on the time from the onset of QRS or ventricular pacing pulse to the onset of right carotid arterial flow, and the PEP^{lc} at 447 represents extended or delayed PEP based on the time from the onset of QRS or ventricular pacing pulse to the onset of left carotid arterial flow.

PTT is calculated through a regression model that assesses the time delay between two sets of BioZ waveforms at each wing. In one embodiment, minor variations in the descending slope of these two waveforms may result in differing delay measurements. Specifically for wing 160, a regression model can be employed to estimate PTT^{p} at 442 based on the time delay between

$$Δ {Z}_{c 1}^{p} ( t ) and Δ {Z}_{c 2}^{p}$$

at three distinct delay points: the beginning

$$( {T}_{n}^{p} ) ,$$

the middle

$$( {T}_{m}^{p} ) ,$$

and the end

$$( {T}_{p}^{p} )$$

of the descending slopes.

$${T}_{m}^{p}$$

is calculated as the delay from the MDS point of

$$Δ {Z}_{c 1}^{p} ( t ) to Δ {Z}_{c 2}^{p} , while {T}_{n}^{p} and {T}_{p}^{p}$$

and are the delays between the negative and positive peaks of

$${\partial }^{2} {Z}_{c 1}^{p} / \partial {t}^{2} and {\partial }^{2} {Z}_{c 2}^{p} / \partial {t}^{2} .$$

Likewise, for wing 161, the flow computes PTT^{rc} at 445 based on the time delay between

$$Δ {Z}_{c 1}^{r c} ( t ) and Δ {Z}_{c 2}^{r c} ( t )$$

at three distinct delay points: the beginning

$$( {T}_{n}^{rc} ) ,$$

the middle

$$( {T}_{m}^{rc} ) ,$$

and the end

$$( {T}_{p}^{rc} )$$

of the descending slopes.

$${T}_{m}^{rc}$$

is calculated as the delay from the MDS point of

$$Δ {Z}_{c 1}^{r c} ( t ) to Δ {Z}_{c 2}^{r c} ( t ) , while {T}_{n}^{rc} and {T}_{p}^{rc}$$

are the delays between the negative and positive peaks of

$${\partial }^{2} {Z}_{c 1}^{r c} / \partial {t}^{2} and {\partial }^{2} {Z}_{c 2}^{r c} / \partial {t}^{2} .$$

For wing 162, the flow computes PTT^{lc} at 448 based on the time delay between

$$Δ {Z}_{c 1}^{l c} and Δ {Z}_{c 2}^{lc}$$

at three distinct delay points: the beginning

$$( {T}_{n}^{lc} ) ,$$

the middle

$$( {T}_{m}^{lc} ) ,$$

and the end

$$( {T}_{p}^{lc} )$$

of the descending slopes.

$${T}_{m}^{lc}$$

is calculated as the delay from the MDS point of

$$Δ {Z}_{c 1}^{lc} to Δ {Z}_{c 2}^{lc} , while {T}_{n}^{lc} and {T}_{p}^{lc}$$

are the delays between the negative and positive peaks of

$${\partial }^{2} {Z}_{c 1}^{lc} / \partial {t}^{2} and {\partial }^{2} {Z}_{c 2}^{lc} / \partial {t}^{2} .$$

Regarding LVET, it is determined by the time interval between the B and X points on the first derivative of the BioZ waveforms. In one embodiment, the LVET^{p} at 443 denotes the average time interval between the B and X points on

$$\partial {Z}_{c 1}^{p} / \partial t and \partial {Z}_{c 2}^{p} / \partial t .$$

Similarly, the LVET^{rc} at 446 represents the average time interval from the B point to the X point on

$$\partial {Z}_{c 1}^{r c} / \partial t and \partial {Z}_{c 2}^{r c} ( t ) / \partial t ,$$

and the LVET^{lc} at 449 represents the average time interval between the B point and the X point on

$$\partial {Z}_{c 1}^{lc} / \partial t and \partial {Z}_{c 2}^{l c} / \partial t .$$

The extended PEP may be adjusted to the corrected PEP (i.e., PEP_{c}) by subtracting the pulse transit time (PTT). PTT represents the duration of ventricular isometric contraction, whereas PEP_{c} represents a true PEP devoid of any temporal delay. In a particular embodiment, PEP_{c} signifies the extended PEP that has been thoroughly corrected for the selected QRS type or left ventricular (LV) pacing mode without any intra-vascular delay (IVD). In this context, PEP_{c} is defined by the equation: PEP_{c}=PEP−PTT. When a finite IVD is present, PEP_{c} may be represented by the equation PEP_{c}=PEP−PTT−IVD, where IVD=0.38×QRS_{d}−8.

In one particular implementation, a heart rate-corrected left ventricular ejection time (LVET_{c}) is derived from heart rate (HR) and LVET. The heart rate-corrected LVET is represented by the equation LVET_{c}=LVET+k (HR−HR_{b}), wherein k denotes the slope of the LVET relative to HR. The precise value of k can be determined as the ratio of the change in LVET that occurs due to a variation in HR_{∞} with the baseline HR calculated as HR_{b}=60,000-72,000/cycle length (milliseconds). Correcting the LVET for HR significantly improves the precision of comparisons made within the same individual at varying heart rates or in relation to other individuals.

At stage 5, the flow computes the PTT-corrected PEP, denoted as

$$P E {P}_{c}^{P}$$

at 451, and HR-corrected LVET, denoted as

$$L V E {T}_{c}^{p}$$

at 452, for wing p, using:

$$P E {P}_{c}^{p} = P E {P}^{p} - P T {T}^{p} - IVD \qquad ( 11 )$$

$${LVET}_{c}^{p} = L V E {T}^{p} + k ( HR - {HR}_{b} ) \qquad ( 12 )$$

### Estimated

$$L V E {T}_{c}^{p}$$

from stage 5 may be used to calculate stroke volume (SV). Likewise, the flow computes the PTT-corrected PEP, denoted as

$$P E {P}_{c}^{r c}$$

at 453 and HR-corrected LVET, denoted as

$$L V E {T}_{c}^{r c}$$

at 454, for wing r using:

$$P E {P}_{c}^{r c} = P E {P}^{r c} - P T {T}^{r c} - IVD \qquad ( 13 )$$

$${LVET}_{c}^{r c} = L V E {T}^{r c} + k ( HR - {HR}_{b} ) \qquad ( 14 )$$

and the PTT-corrected PEP, denoted as

$$P E {P}_{c}^{lc}$$

at 455 and HR-corrected LVET, denoted as

$$LVE {T}_{c}^{lc}$$

at 456, for wing l using:

$$P E {P}_{c}^{lc} = P E {P}^{lc} - P T {T}^{lc} - IVD \qquad ( 15 )$$

$$LVE {T}_{c}^{lc} = L V E {T}^{lc} + k ( HR - {HR}_{b} ) \qquad ( 16 )$$

At stage 6, left ventricular ejection fraction (LVEF) is estimated at 461. The LVEF is estimated using, in one embodiment, Chirfle's formula. For example, LVEF can be estimated as follows:

$$LVEF = ( {k}_{1} {Log}_{e} ( {LVET}_{c}^{p} / {PEP}_{c}^{p} ) + {k}_{2} ) \times 100 \qquad ( 17 )$$

where
k₁ and k₂ are constants,

$${LVET}_{c}^{p}$$

is the HR-corrected LVET,
and

$${PEP}_{c}^{p}$$

is the PTT-corrected PEP for wing p.
   Alternatively, LVEF is estimated using Weissler's Regression. For example, LVEF can be estimated as follows:

$$LVEF = {k}_{3} - {k}_{4} {PEP}_{c}^{p} / {LVET}_{c}^{p} \qquad ( 18 )$$

The constants k₁, k₂, k₃, and k₄ used in Equations (17) and (18) can be obtained by comparing

$${PEP}_{c}^{p} / {LVET}_{c}^{p}$$

(Weissler's ratio) with LVEF measured using echocardiography (ECG) or MRI or any other Gold-Standard devices.

Arterial stiffness, quantified by arterial pulse wave velocity (PWV), is a recognized biomarker indicative of cardiovascular risk and target organ damage among individuals with hypertension. In one embodiment, PWV at the right and left carotid artery determined at 462 and 463 can be defined as:

$${PWV}^{r c} = {D}^{r c} / {PTT}^{r c} \qquad ( 19 )$$

$${PWV}^{l c} = {D}^{lc} / {PTT}^{lc} \qquad ( 20 )$$

where
   D^{rc} dis the distance between

$$( {V}_{1}^{r c} + , {V}_{1}^{r c} - ) and ( {V}_{2}^{r c} + , {V}_{2}^{r c} - )$$

in wings r, and
D^{lc} is the distance between

$$( {V}_{1}^{lc} + , {V}_{1}^{lc} - ) and ( {V}_{2}^{lc} + , {V}_{2}^{lc} - )$$

in wings l.

Regarding SV, it is defined as the volume of the blood pumped from the left ventricle of the heart in one cardiac cycle and expressed as the difference between the left ventricular end-diastolic volume (LVEDV) and left ventricular end-systolic volume (LVESV).

At stage 7, the flow computes SV at 471 by measuring the first derivative of

$$B i o {Z}_{1}^{p} ( t )$$

to calculate base impedance

$${Z}_{0}^{p} and {( \partial {Z}_{c 1}^{p} / \partial t )}_{max} .$$

For example, SV can be calculated as:

$$SV = ρ ( {L}^{2} / {Z}_{0}^{p} ) \frac{{( \partial {Z}_{c 1}^{p} / \partial t )}_{max}}{( {Z}_{0}^{p} / {LVET}_{c}^{p} )} \qquad ( 21 )$$

where
ρ is the resistivity of blood,
L is the distance between the voltage-sensing electrodes

$${V}_{1}^{p} + and {V}_{1}^{p} -$$

in wing p,

$${Z}_{0}^{p}$$

is the base impedance,

$${( \partial {Z}_{c 1}^{p} / \partial t )}_{max}$$

is the absolute value of the cyclic peak of the first derivative of the cardiac component

$${Z}_{c 1}^{p} of {BioZ}_{1}^{p} ,$$

and
LVET_{c}^{p} is HR-corrected left ventricular ejection time in wing p.

Once PWV^{rc} and PWV^{lc} are calculated, the flow at stage 7 estimates the arterial pressures CAP^{rc} at 472 in the right carotid artery and CAP^{lc} at 473 in the left carotid artery using:

$${CAP}^{rc} = {α}_{1} {PWV}^{{rc}^{2}} + {β}_{1} \qquad ( 22 )$$

$${CAP}^{lc} = {α}_{2} {PWV}^{{lc}^{2}} + {β}_{2} \qquad ( 23 )$$

The constants α₁, β₁, α₂, and β₂ used in Equations (22) and (23) depend on the material properties and geometry of the right and left carotid arteries and are to be determined from the experiments.

At stage 8, left ventricular end-diastolic pressure (LVEDP) is calculated at 482 or 483. In one embodiment, LVEDP, often called preload, is the amount of ventricular stretch at the end of diastole and may be determined using a “cardiac triangle” technique. For example, a triangular relationship among the relevant parameters can be established using arterial diastolic pressure (AoDP), pre-ejection period (PEP), contractility (Ctr), and left ventricular end-diastolic pressure (LVEDP). In a particular embodiment, LVEDP is calculated using:

$$LVEDP = {c}_{1} {CADP}^{rc} - {c}_{2} ( LVEF \times {PEP}_{c}^{rc} ) / √ {LVET}_{c}^{rc} \qquad ( 24 )$$

$$LVEDP = {c}_{3} {CADP}^{lc} - {c}_{4} ( LVEF \times {PEP}_{c}^{lc} ) / √ {LVET}_{c}^{lc} \qquad ( 25 )$$

where
c₁ and c₃ are unitless constants,
c₂ and c₄ are constants with a unit of mmHg/√Second, and
the constants c₁, c₂, c₃, and c₄ can be obtained, for example, using linear multiple regression.

In yet another embodiment, LVEDP can be calculated using:

$$LVEDP = [ {c}_{5} ( MAP ) \times ( 1 - LVEF ] - {c}_{6} \qquad ( 26 )$$

where
MAP is the mean arterial pressure measured at the common carotid artery,
c₃ is a unitless constant, and
c₄ is a constant with a unit of mmHg.
   This appears to be the most accurate, reliable, and easily applied technique for estimating LVEDP noninvasively in patients with preserved ejection fraction and an LVEDP <20 mm Hg.

At stage 8, ECF is calculated at 481 using Cole-Cole parameter R₀ is based on Hanai's mixture theory, which accounts for the effect of non-conducting particles (cells) suspended in a conductive medium and is expressed as:

$$ECF = {{k}_{ECF} ( {W}^{0.5} {H}^{2} / {R}_{0} )}^{2 / 3} \qquad ( 27 )$$

where
k_{ECF} is the Extracellular fluid constant,
W is the body weight (kg), and
H is the height (cm).
   The constant k_{ECF} is calculated from the physical properties of ECF and the body geometry:

$${k}_{ECF} = {k}_{B}^{2 / 3} {ρ}_{e}^{2 / 3} / {D}_{b}^{2 / 3} \qquad ( 28 )$$

where
k_{B} is the Body shape factor (dimensionless constant relating height to limb and trunk dimensions),
ρₑ is the Extracellular fluid resistivity (Ω·cm), and
D_{b} is the Body density (kg/m³).
   Clinical studies have established standardized values for these parameters. The extracellular fluid resistivity (ρ_{lc}) has been determined to be ≈66.9±1.8 Ω·cm for men and ≈67 Ω·cm for women, closely matching the resistivity of saline solutions that approximate plasma and interstitial fluid composition.

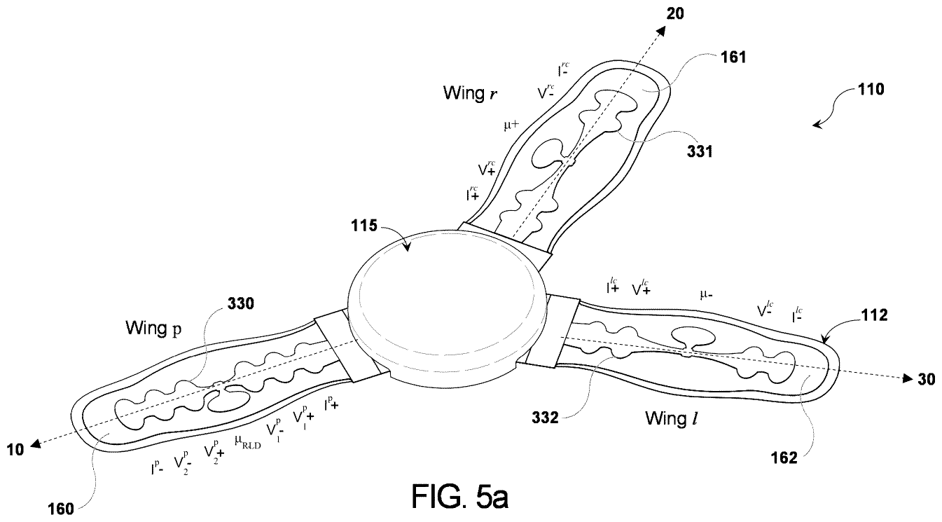

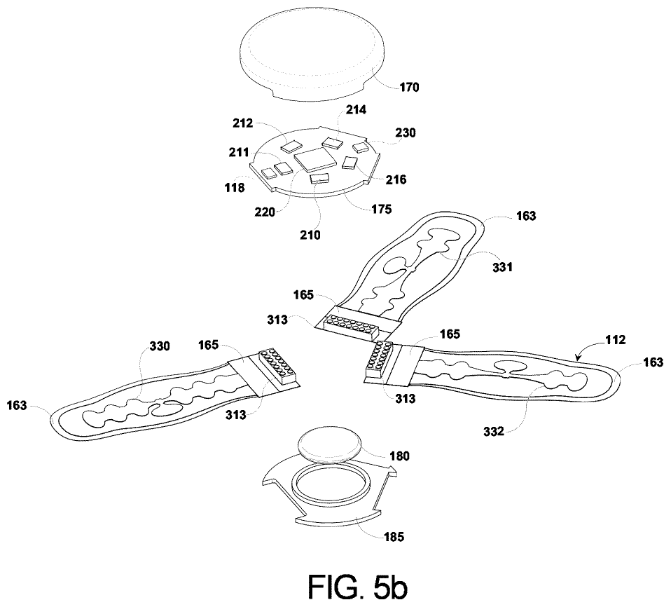

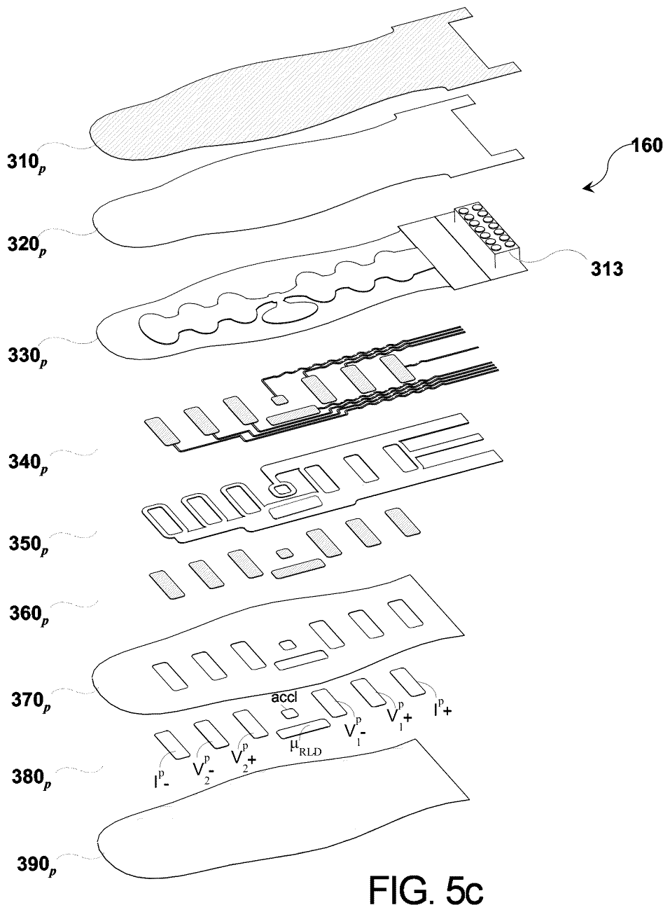

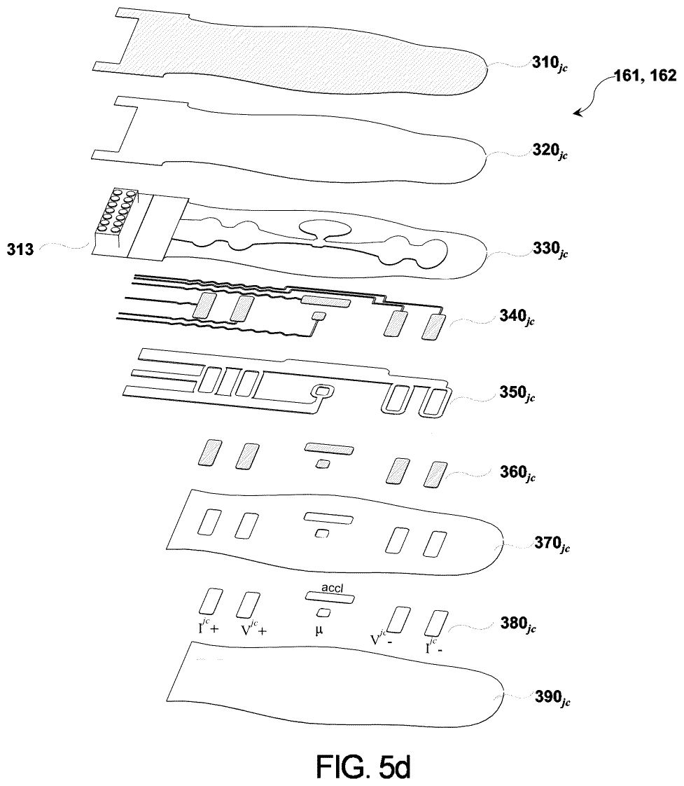

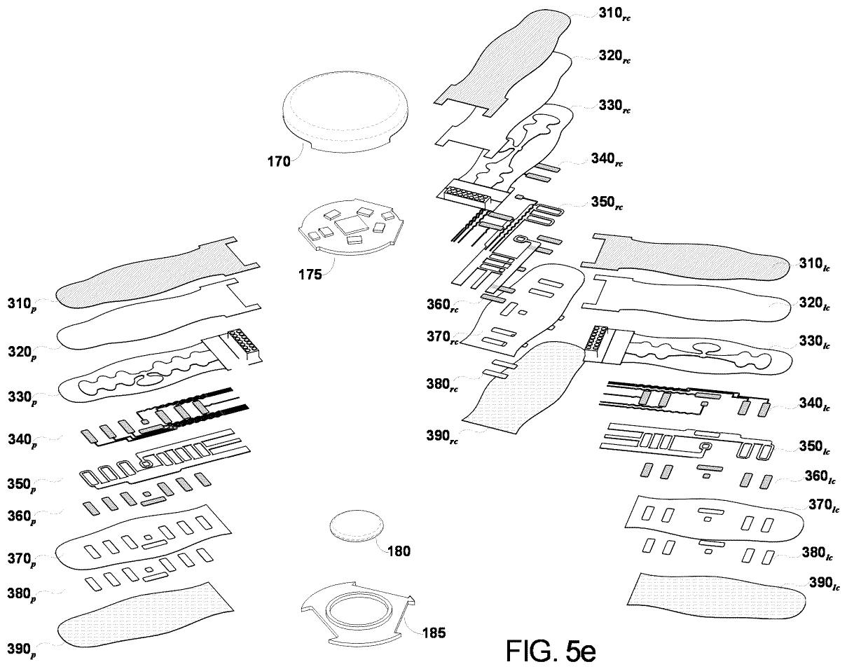

FIGS. 5a-5ₑ shows various views of another embodiment of a wearable hemodynamic monitoring device 110. In particular, FIGS. 5a and 5b show 3D perspective and partially exploded views, respectively, FIG. 5_{lc} shows a 3D exploded view of the bioimpedance measuring wing 160, FIG. 5d shows a 3D exploded view of the bioimpedance measuring wings 161 and 162, and FIG. 5e shows a fully exploded view of the hemodynamic monitoring device 110, illustrating the components of the flexible body 112 and the rigid housing 115. As shown, the wearable device is a patch that includes various components for monitoring HF events in a user. The wearable device 110 is similar to that described in FIGS. 2a-2d. Common elements may not be described or described in detail.

In one embodiment, the hemodynamic monitoring device 110 includes a flexible body 112 connected to a water-tight and rigid housing 115. The flexible body 112 is designed to conform to the user's body contour. The flexible body 112, as shown, includes multiple bioimpedance wings. In one embodiment, the flexible body includes a primary bioimpedance measuring wing 160 placed on the chest and two identical bioimpedance measuring wings 161 and 162 worn on the neck. The wings of the flexible body feature ultrathin, stretchable substrate layers 330, 331, and 332 that extend laterally from the rigid housing 115. The first wing 160 is positioned on the midsternal region of the upper thorax. The first neck-worn wing 161 is located on the right side of the neck over the right common carotid artery (RCCA) and the second neck-worn wing 162 is on the left side of the neck over the left common carotid artery (LCCA).

Each wing contains a set of flexible serpentine traces that are embedded within the wings and are mechanically isolated from the rigid housing. Each electrode trace is attached to the bottom surface of the flexible body 112 using a flexible electrode, and each sensor trace is connected to an accelerometer sensor on the same surface. This design enables the sensors and electrodes to make conformal contact with the user's skin, thereby facilitating the detection of both accelerometer and hemodynamic signals. The traces transmit electrical signals from the sensors and electrodes to the PCB assembly 175. Each wing is attached to the rigid housing using a magnetic mating pad connector 313 or another suitable flexible interconnection method. The rigid housing also includes a power source, such as one or more batteries 180.

In one embodiment, the flexible wings 160, 161, and 162 are strategically positioned to encompass the electrode locations for detecting ECG/EKG, bioimpedance and acceleration waveforms. This arrangement allows for the capture of both atrial and ventricular activity. The role also includes overseeing the placement of sensors to capture hemodynamic activity in the descending aorta and monitoring the lung filling cycle.

In one of the embodiments, a first array of electrodes and sensors is embedded in the primary bioimpedance measuring wing 160, wherein the first array includes a first (I^{p}+) and second (I^{p}−) electrode, positioned along the midsternal line 10 on the upper and lower aspects of the wing. The first and second electrodes of the first array are specifically designed for current injection into the upper thoracic region of the user's body. A third

$$( {V}_{1}^{p} + )$$

and a fourth

$$( {V}_{1}^{p} - )$$

electrode of the first array are positioned along the same line 10, with the third electrode located below the positive current-injecting electrode I^{p}+ and the fourth electrode positioned below the third. The third

$$( {V}_{1}^{p} + )$$

and fourth

$$( {V}_{1}^{p} - )$$

electrodes are configured to detect voltage at a specified location on the upper thoracic region of the user's body.

Furthermore, a fifth

$$( {V}_{2}^{p} + )$$

and a sixth

$$( {V}_{2}^{p} - )$$

electrode of the first array are located along the aforementioned line 10, with the fifth electrode positioned below the fourth electrode and the sixth electrode placed between the negative current-injecting electrode I^{p}− and the fifth electrode. The fifth

$$( {V}_{2}^{p} + )$$

and sixth

$$( {V}_{2}^{p} - )$$

electrodes of the first array are configured to detect voltage at a different location on the upper thoracic region of the user's body.

An accelerometer sensor is placed between the fourth

$$( {V}_{1}^{p} - )$$

and fifth

$$( {V}_{2}^{p} + )$$

electrodes for detecting motion signals along three axes. A seventh electrode (μ_{RLD}), positioned along a line perpendicular to the midsternal line 10 on the right side of the accelerometer sensor. The seventh electrode, along with electrode (μ+) in wing 161 and electrode (μ−) in wing 162, is configured to detect an ECG/EKG signal from the human body.

In the same embodiment, a second array of electrodes and sensors is embedded in the first neck-worn wing 161, wherein the second array includes a first (I^{rc}+) and second (I^{rc}−) electrode. The first and second electrodes of the second array are configured to be located approximately along the right common carotid artery (RCCA) 20 on the upper and lower aspects of the wing. The first and second electrodes of the second array are specifically designed for current injection into the user's right neck. A third (V^{rc}+) and a fourth (V^{rc}−) electrode of the second array are configured for an approximate location along the RCCA 20, with the third electrode located below the first current-carrying electrode and the fourth electrode placed above the second current-carrying electrode. The third (V^{rc}+) and fourth (V^{rc}−) electrodes are configured to detect voltage at a specified location on the RCCA of the user's body. Unlike the wearable patch depicted in FIGS. 2a-2d, the second array does not incorporate the fifth and sixth electrodes.

An accelerometer sensor located between the third and fourth electrodes can detect motion signals along three axes. Additionally, a fifth electrode (μ+) is arranged along a line perpendicular to RCCA 20 on the right side of the accelerometer sensor. The fifth electrode, along with electrode (μ−) in wing 162 and electrode (μ_{RLD}) in wing 160, is configured to detect an ECG/EKG signal from the human body.

In the same embodiment, a third array of electrodes and sensors is embedded in the second neck-worn wing 162, wherein the second array includes a first (I^{lc}+) and second (I^{lc}−) electrode, configured for an approximate location along the left common carotid artery (LCCA) 30 on the upper and lower aspects of the wing. The first and second electrodes of the third array are specifically designed for current injection into the user's left neck. A third (V^{lc}+) and a fourth (V^{lc}−) electrode of the third array are configured for an approximate location along the LCCA 30, with the third electrode located below the first current-carrying electrode and the fourth electrode placed above the second current-carrying electrode. The third (V^{lc}+) and fourth (VI^{lc}−) electrodes are configured to detect voltage at a specified location on the LCCA of the user's body. Like the second array of electrodes, the third array does not incorporate a fifth and sixth electrode.

An accelerometer sensor is positioned between the third and fourth electrodes and can detect motion signals along three axes. Additionally, a seventh electrode (μ−), arranged along a line perpendicular to LCCA 30, on the right side of the accelerometer sensor, along with electrode (μ+) in wing 161 and electrode (μ_{RLD}) in wing 160, is configured to detect an ECG/EKG signal from the human body.

In one embodiment, the rigid housing 115 includes an upper housing member 170 that can be easily connected and disconnected from a lower housing member 185. Several components of the monitoring device 110 are located between the upper and the lower housing members. For example, the rigid housing 115 may encompass a section of the flexible body 112, a signal acquisition unit (SAU) 175 in the form of a PCB assembly, a battery 180, and spring connectors. The SAU 175 is placed inside housing 115 to establish contact with sensors, electrode traces, and battery 180 via spring connectors and mating pad connectors on both the PCB and the traces, respectively. Referring to FIG. 5b, the SAU 175 may include a plurality of AFE circuits 210, 211, 212, 214, 216, a processor 220, a memory unit 230, a wireless transmitter module 118, and a power supply 180. One of the AFEs is used to acquire a 1-lead EKG/ECG waveform, while the other AFEs are used to measure impedance plethysmography signals from each wing. For instance, signals from the electrode (μ_{RLD}) in wing 160, electrode (μ+) in wing 161, and electrode (μ−) in wing 162 are used to measure the electrical activities of the user's heart that is acquired by the ECG/EKG AFE 216 and digitized to generate an ECG/EKG waveform.

The bioimpedance measurement process in Wing 160 begins with the initialization of the SPI communication protocol. The microcontroller is configured as the SPI master, and separate chip select (CS) lines are assigned for AFE 210 and AFE 211. This setup allows for individual control of both Analog Front Ends (AFEs) over the SPI bus. An excitation current pulse from bioimpedance AFE 210 is administered to the user's upper thoracic region at time t=T₀ through the I^{p}+ and I^{p}− electrodes. Simultaneously, the current injection from bioimpedance AFE 211 is disabled by writing to its control register. This is accomplished by selecting AFE 211 (e.g., setting its CS line low), sending the command to turn off the current injection, and then deselecting AFE 211 (e.g., setting its CS line high). At this point, AFE 210 is configured for both current injection and voltage measurement, while AFE 211 is set up solely for voltage measurement. Consequently, AFE 210 generates the excitation current while both AFEs measure the voltage response. AFE 210 is set to output its clock to AFE 211 to synchronize the measurement cycles of the two AFEs. The SPI command of the microcontroller is configured to generate this signal, ensuring both AFEs commence their measurement cycles simultaneously. This synchronization generates a trigger signal that initiates the measurement processes, allowing AFE 210 to provide the excitation current while both AFEs capture the voltage response. This synchronized measurement yields accurate and correlated data from both AFEs. The bioimpedance measurement data is read from AFE 210 and AFE 211.

The excitation current waveform generated by the bioimpedance AFE 212 is injected into the right common carotid artery of the user via I^{rc}+ and I^{rc}− electrodes in wing 161, while the excitation current waveform generated by the bioimpedance AFE 214 is injected into the left common carotid artery of the user via I^{lc}+ and I^{lc}− electrodes in wing 162 respectively. The processor 220 is configured to preprocess the input data from the AFEs, the wireless transmitter module 118, and the accelerometer sensors. The wireless module 118 communicates wirelessly with a smartphone 120 and a web server 140 using Bluetooth, Wi-Fi, or other wireless communication standards. The preprocessed data may be forwarded to the user's mobile device for further interpretation by the wireless transmission module. The preprocessed data can also be stored in the memory unit as needed.

Referring now to FIG. 5c, there is shown a comprehensive fully exploded view of the flexible body corresponding to the wing 160, illustrating the complete component hierarchy and multi-layered construction that enables the integrated chest-worn cardiovascular monitoring capabilities. This exploded view demonstrates the sophisticated layered architecture comprising both the rigid housing assembly and the flexible body construction, revealing the individual components and their spatial relationships within the overall device structure. It includes a top fabric layer 310ₚ, a low-temperature hot melt adhesive layer 320ₚ, a substrate layer 330ₚ, a series of sensor and electrode trace layers 340ₚ, 350ₚ, 360ₚ, an adhesive layer 370ₚ, an array of six bioimpedance and one EKG/ECG electrodes along with an accelerometer sensor 380ₚ, and a release liner 390ₚ. Similarly, FIG. 5d illustrates the 3D exploded view of the identical bioimpedance wings 161 and 162 comprising a top fabric layer 310_{jc}, a low-temperature hot melt adhesive layer 320_{jc}, a substrate layer 330_{jc}, a series of sensor and electrode trace layers 340_{jc}, 350_{jc}, 360_{jc}, an adhesive layer 370_{jc}, an array of six bioimpedance and one EKG/ECG electrodes along with an accelerometer sensor 380_{jc}, and a liner 390c, j=r, l.

As shown in FIGS. 5_{lc} and 5d, the upper layer 310ₖ (k=p, rc, lc) is made from elastomeric compression fabric, combining materials like polyester with LYCRA, nylon, or cotton-polyester rib. The low-temperature hot melt layer 320ₖ uses a thermoplastic double-sided adhesive film from polyurethane, which is white and translucent, ideal for bonding textiles and sponges. A TPU hot melt adhesive web may also be employed, which is non-sticky at room temperature and bonds upon heating yet remains stable at low temperatures. An alternative option for the upper layer is non-woven textile material 310ₖ coated with polymers 330ₖ, such as polyurethane, nylon, and thermoplastic polyurethane (TPU). The electrode and sensor trace materials may involve layers of stretchable silver (Ag) ink 340ₖ and silver chloride (AgCl) ink 360ₖ, separated by dielectric ink 350ₖ. Conductive traces are interconnected through openings in the dielectric layer. To protect printed conductive traces 340ₖ and 360ₖ on the PCB, a dielectric layer 350ₖ is applied to reduce the risk of silver migration, preventing short circuits. An alternate embodiment of the conductive trace materials may involve conductive textile materials for both layers, separated by a dielectric layer. For instance, a stretchable fabric like Shieldex® “P130+B” may be used.

FIGS. 5_{lc} and 5d show a skin adhesive layer 370ₖ (k=p, rc, lc) with empty spaces corresponding to the electrodes and sensors. The adhesive should ensure long-term, comfortable adhesion for 14-30 days, potentially using a stretchable, non-woven fabric coated with a pressure-sensitive acrylic adhesive. In an embodiment, the adhesive material can be made of ARcare® 93690, ARcare® A-5490 or materials with similar properties. Each adhesive layer 370ₖ (k=p, rc, lc) is designed with specific voids to accommodate either an electrode or an accelerometer sensor 380ₖ. The accelerometer may utilize a 3-axis inertial measurement unit (IMU), such as BMI270. The flexible electrodes are formulated from materials such as hydrogel. Alternatively, the electrodes and adhesive can be replaced with a conductive layer, allowing the entire adhesive underside to function as an electrode. This hybrid adhesive and conductive layer may include materials similar to a hydrogel-silicone adhesive blend. In another embodiment, the electrodes 380ₖ may be constructed from conductive textile materials, including the conductive Shieldex® “P130+B.” Additionally, electrodes may also incorporate carbon-enhanced adhesive materials. The wearable monitor may feature a protective release liner 390ₖ, which ensures structural integrity, cleanliness, and security during application. This liner can be affixed to the bottom surface of the adhesive layer 370ₖ to facilitate the accurate placement of the wearable device 110 on the user. Furthermore, the liner 390ₖ typically has an adhesive side that adheres to the bottom surfaces of the wings on the flexible body 112.

FIG. 5ₑ illustrates a fully exploded view of the hemodynamic monitoring device 110, showcasing the components that constitute the flexible body 112 and the rigid housing 115. In this configuration, the rigid housing 115 has an upper housing member 170, which can be readily attached to or detached from a lower housing member 185. This housing may incorporate a segment of the flexible body 112, a signal acquisition unit (SAU) 175, a battery 180, and three spring connectors referred to as magnetic Pogo Pins, which are not depicted in FIG. 5b. The flexible body 112, in one embodiment, includes a bioimpedance-measuring wing 160 with two current-injection and four voltage detection electrodes placed on the chest and two identical bioimpedance-measuring wings 161 and 162 with two current-injection and two voltage detection electrodes worn on the neck. The first bioimpedance wing 160 contains a top fabric layer 310ₚ, a low-temperature hot melt adhesive layer 320ₚ, a substrate layer 330ₚ, a series of sensor and electrode trace layers 340ₚ, 350ₚ, and 360ₚ, an adhesive layer 370ₚ, an array of six bioimpedance electrodes, an ECG/EKG electrode, and an accelerometer sensor 380ₚ, in addition to a liner 390ₚ. Similarly, the second bioimpedance wing 161 includes a top fabric layer 310_{rc}, a low-temperature hot melt adhesive layer 320_{rc}, a substrate layer 330_{rc}, a series of sensor and electrode trace layers 340_{rc}, 350_{rc}, and 360_{rc}, an adhesive layer 370_{rc}, an array of flexible electrodes, and an accelerometer sensor 380_{rc}, along with a liner 390_{rc}. The third bioimpedance wing 162 follows the same structural format as wing 161 with its corresponding top fabric layer 310_{lc}, low-temperature hot melt adhesive layer 320_{lc}, substrate layer 330_{lc}, sensor and electrode trace layers 340_{lc}, 350_{lc} and 360_{lc}, adhesive layer 370_{lc}, an array of six bioimpedance electrodes, an ECG/EKG electrode, and an accelerometer sensor 380_{lc}, in conjunction with a liner 390_{lc}.

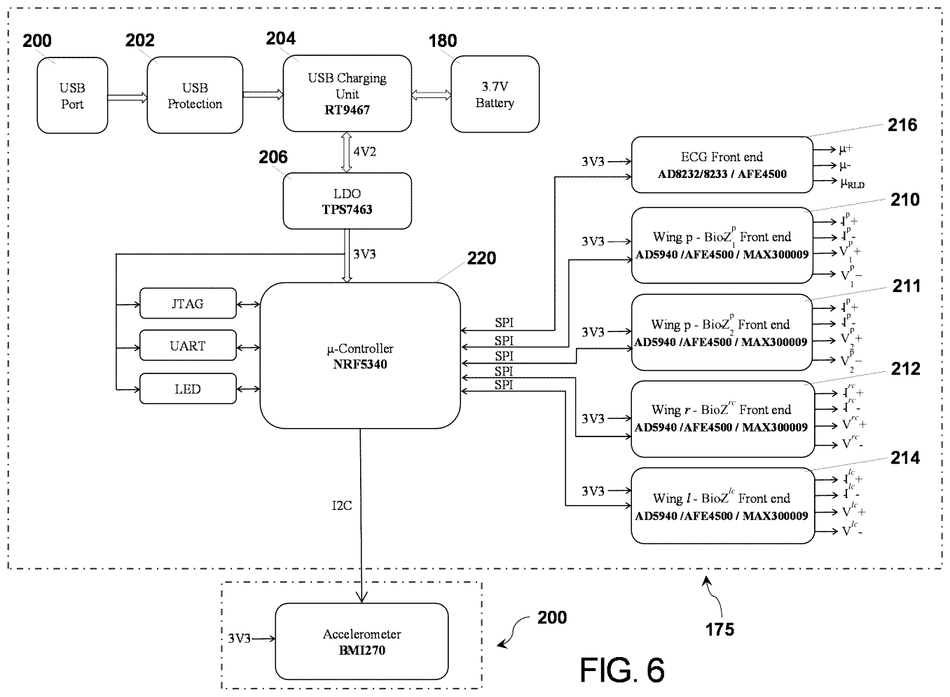

FIG. 6 illustrates an embodiment of a PCB utilized for the signal acquisition unit (SAU) 175 and wing x PCB 200 (x=160, 161, and 162). The SAU-PCB houses an array of digital circuits and subsystems essential for the functionalities of wearable devices. Although the SAU is optimally integrated within the hemodynamic monitoring device 110, it could also be located separately. Principal components of the SAU-PCB include a USB port 202, a USB protection module 204, a USB power module 206, a battery module 180, an LDO 208, four analog front-end circuits 210, 211, 212, and 214 for receiving data from bioimpedance electrodes, one analog front-end circuit 216 for receiving data from ECG/EKG electrodes and a microcontroller 220. The USB port facilitates device connectivity, while the protection module mitigates risks associated with power surges. The USB power module contributes to the charging of the battery, which may be a 3.7V (1 amp) variant or an alternative suitable option. The power converter supervises voltage regulation to energize various subsystems and may incorporate a Low Drop-Out (LDO) regulator, such as TPS746 produced by Texas Instruments, USA.

The analog front-end circuits 210 and 211 can have multiple modulation and demodulation circuits to measure impedance at two different locations along the midsternal line in the upper thoracic region. In contrast, analog front-end circuits 212 and 214 are used to measure bioimpedance at the neck regions around the right and left carotid arteries, respectively. The Analog front-end circuit 216 may have various amplifier and filter circuits to measure an ECG/EKG waveform. All analog front-end circuits and the front-end circuit for 6-axis accelerometer 200 are connected to the processing unit 220 to allow the processing of the signals. A power supply module 180 guarantees adequate voltage and power, while the analog circuits translate analog sensor signals into a digital format. These components are sourced from various manufacturers, including Texas Instruments and Microchip Technology. The SAU 175 integrates memory for the microcontroller's software and sensor data preservation.

It may also include integrated memory options, such as the nRF5340 from Nordic Semiconductor. Furthermore, an in-built wireless communication module facilitates communication with smartphones 120 and web servers 140 through Bluetooth or Wi-Fi.

The operational principle of the wearable monitor in FIG. 5a is the same as that of the wearable monitor in FIG. 2a. The signal processing unit 175 is programmed to simultaneously activate three different patch wings: 160, 161, and 162. This enables remote monitoring of deteriorating hemodynamic parameters in heart failure patients.

Once the wearable monitoring device 110 is activated, real-time impedance waveforms are generated simultaneously for each wing:

For wing p:

$$B i o {Z}_{1}^{p} = ( {V}_{1}^{p} + - {V}_{1}^{p} - ) / ( {I}^{p} + - {I}^{p} - ) \qquad ( 29 a )$$

$$Bio {Z}_{2}^{p} = ( {V}_{2}^{p} + - {V}_{2}^{p} - ) / ( {I}^{p} + - {I}^{p} - ) \qquad ( 29 b )$$

For wing r:

$$B i o {Z}^{r c} = ( {V}^{r c} + - {V}^{r c} - ) / ( {I}^{r c} + - {I}^{r c} - ) \qquad ( 29 c )$$

For wing l:

$$B i o {Z}^{lc} = ( {V}^{lc} + - {V}^{lc} - ) / ( {I}^{lc} + - {I}^{lc} - ) \qquad ( 29 d )$$

In addition to the aforementioned impedance measurements, the device also acquires real-time ECG/EKG data and three-axis acceleration waveforms. Subsequent to transmitting these real-time waveforms to a web-based server via an IoT gateway, a deep learning model is activated to forecast these waveforms several days to months in advance. As described earlier, the predicted

$$B i o {Z}_{1}^{p} and {BioZ}_{2}^{p}$$

facilitate measuring extracellular fluid (ECF), left ventricular ejection fraction (LVEF), stroke volume (SV) and cardiac output (CO) at the pulmonary region. However, PTT-based carotid arterial pressure (CAP) monitoring methods depend on generalized expert features that may not exhibit consistent behavior across individuals. The carotid arterial waveform shows dynamic intra-subject changes due to aging, carotid artery stiffness, and other underlying factors influencing arterial dynamics, which are not adequately formalized. Consequently, machine learning algorithms mapping features to CAP are trained on large bioimpedance datasets to account for inter-subject variability and should undergo frequent calibration for intra-subject changes. The bioimpedance waveforms BioZ^{rc} and BioZ^{lc} measured across right and left carotid arteries are used to measure arterial pressure pulse at the right and the left carotid arteries, respectively.

The features extracted at each heartbeat window of pre-processed waveforms are analyzed using a predictive model based on a neural network (NN) model. When bioimpedance electrodes are placed along the carotid arteries, the acquired BioZ^{rc} and BioZ^{lc} waveforms change quasi-periodically with the change in the artery volume due to the arrival of the carotid pulse wave at each heartbeat cycle. Specifically, a reduction in bioimpedance indicates the arrival of blood through the sensing area on the carotid arteries, a phase referred to as systolic, during which the left ventricle of the heart contracts to propel blood through the carotid arteries. Conversely, the phase characterized by increased impedance is identified as the diastolic phase, during which the heart undergoes a resting period between beats. The volumetric change associated with each cardiac cycle correlates with the pressure exerted on the carotid arterial walls.

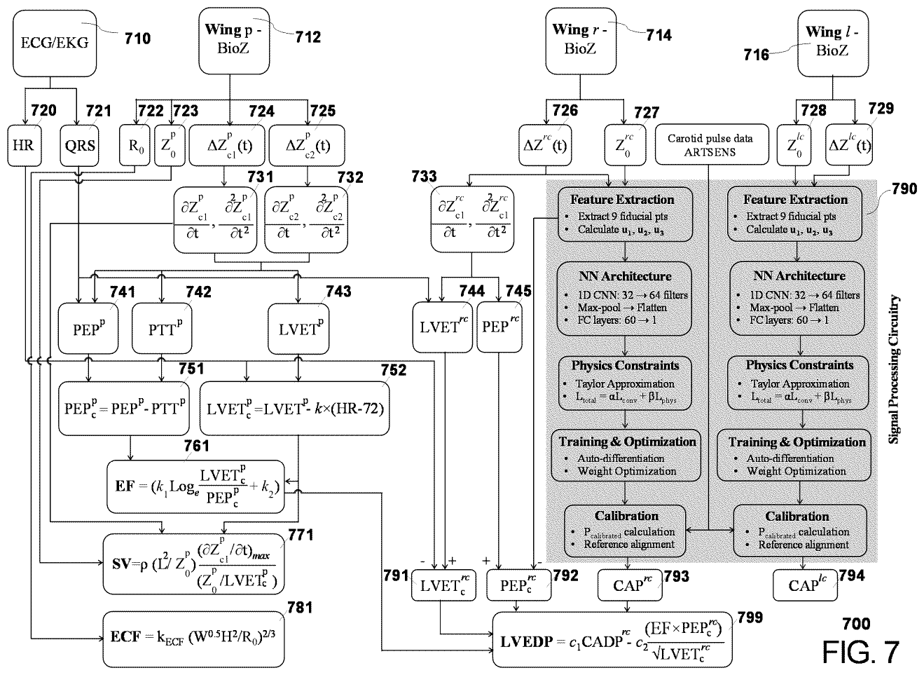

FIG. 7 illustrates a software flow 700 for extracting clinical parameters from data recorded by the sensors and electrodes of the wearable device. This wearable device is akin to that depicted in FIGS. 2a-2d. In one embodiment, a cloud server carries out the clinical parameter extraction. For instance, preprocessed data from the wearable device is sent to the user's mobile device, which then forwards it to the server. Alternatively, the preprocessed data may be transmitted directly from the wearable device to the server. For example, the main PCB could contain a mobile communication module, such as a Wi-Fi or BLE module, allowing the preprocessed data to be sent to the cloud server via a cellular network. This flow, for instance, may be executed by a software application running on the server.

The flow, for example, may include multiple stages for processing the data collected from the wearable device. The flow consists of eight stages (stages 1 to 8), But providing a flow with other numbers of stages may also be helpful.

At stage 1, physiological data collected from the wearable device is received. For example, stage 1 may be referred to as an input stage. In one embodiment, EKG/ECG waveform from μ+, μ− and μ_{RLD} electrodes are received at 710,

$${BioZ}_{1}^{p} ( {V}_{1}^{p} + , {V}_{1}^{p} - ) and {BioZ}_{2}^{p} ( {V}_{2}^{p} + , {V}_{2}^{p} - )$$

data from the I^{p}+ and I^{p}− electrodes of wing p is received at 712, BioZ^{rc}(V^{rc}+, V^{rc}−) data from the I^{rc}+ and I^{rc}− electrodes of wing r is received at 714, and BioZ^{lc}(V^{lc}+, V^{lc}−) data from the I^{lc}+ and I^{lc}− electrodes of wing l is received at 716.

At stage 2, the flow employs the input data at 710 to automatically measure heart rate (HR) at 720 and QRS duration ((QRS_{d}) at 721 from EKG/ECG waveforms. In the same embodiment, the flow measures Cole-Cole impedance parameter R₀ at 722,

$${Z}_{0}^{p}$$

at 723,

$$Δ {Z}_{c 1}^{p} ( t )$$

at 724,

$$Δ {Z}_{c 2}^{p} ( t )$$

at 725 from input

$$B i o {Z}_{1}^{p} ( = {Z}_{0}^{p} + Δ {Z}_{c 1}^{p} + Δ {Z}_{r 1}^{p} ) and {BioZ}_{2}^{p} ( = {Z}_{0}^{p} + Δ {Z}_{c 2}^{p} + Δ {Z}_{r 2}^{p} )$$

waveforms. Further, the flow measures ΔZ^{rc}(t) at 726 and

$${Z}_{0}^{r c}$$

at 727 from the input

$$B i o {Z}^{r c} ( = {Z}_{0}^{r c} + Δ {Z}^{r c} )$$

waveform and

$${Z}_{0}^{lc}$$

at 728 and ΔZ^{lc}(t) at 729 from the input

$$B i o {Z}^{lc} ( = {Z}_{0}^{lc} + Δ {Z}^{lc} )$$

waveform. Other parameters may also be determined at stage 2.

The Cole-Cole impedance parameter R₀ calculated at 722 is used to calculate ECF at 781 at stage 8 using a time-domain fitting approach based on single-frequency DC-biased sinusoidal excitation in wing 160.

At stage 3, the flow computes

$$\partial {Z}_{c 1}^{p} / \partial t$$

(i.e., first derivative of

$${Z}_{c 1}^{p} ( t )$$

with time t) and

$${\partial }^{2} {Z}_{c 1}^{p} / \partial {t}^{2}$$

(i.e., 2^{nd} derivative of

$${Z}_{c 1}^{p} ( t )$$

with time t) at 731,

$$\partial {Z}_{c 2}^{p} / \partial t$$

(i.e., first derivative of

$${Z}_{c 2}^{p} ( t )$$

with time t) and

$${\partial }^{2} {Z}_{c 2}^{p} / \partial {t}^{2}$$

(i.e., 2^{nd} derivative of

$${Z}_{c 2}^{p} ( t )$$

with time t) at 732. The flow also computes ∂Z^{rc}(t)/∂t (i.e., first derivative of Z^{rc}(t) with time t) and ∂²Z^{rc}(t)/∂t² (i.e., 2^{nd} derivative of Z^{rc}(t) with time t) at 733. The flow further computes ∂Z^{lc}(t)/∂t (i.e., first derivative of Z^{lc}(t) with time t) and ∂²Z^{lc}(t)/∂t² (i.e., 2^{nd} derivative of Z^{lc}(t) with time t) (not shown).

At stage 4, the flow assesses PEP^{p} at 741 based on the time from the onset of QRS or ventricular pacing pulse to the onset of central aortic flow. A regression model can be employed to estimate PTTpAt 742, based on the time delay between

$$Δ {Z}_{c 1}^{p} ( t ) and Δ {Z}_{c 2}^{p}$$

at three distinct delay points: the beginning

$$( {T}_{n}^{p} ) ,$$

the middle

$$( {T}_{m}^{p} )$$

and the end

$$( {T}_{p}^{p} )$$

of the descending slopes.

$${T}_{m}^{p}$$

is calculated as the delay from the MDS point of

$$Δ {Z}_{c 1}^{p} ( t ) to Δ {Z}_{c 2}^{p} ,$$

while

$${T}_{n}^{p} and {T}_{p}^{p}$$

are the delays between the negative and positive peaks of

$${\partial }^{2} {Z}_{c 1}^{p} / \partial {t}^{2} and {\partial }^{2} {Z}_{c 2}^{p} / \partial {t}^{2} .$$

The flow also calculates LVET^{p} at 743 by measuring the average time interval between the B and X points on

$$\partial {Z}_{c 1}^{p} / \partial t and \partial {Z}_{c 2}^{p} / \partial t$$

waveforms. The flow also computes LVET^{rc} at 744 and PTT^{rc} at 745 for wing r.

At stage 5, the flow computes the PTT-corrected PEP, denoted as

$$P E {P}_{c}^{p}$$

at 751, and HR-corrected LVET, denoted as

$$LVE {T}_{c}^{p}$$

at 752, for wing p using equations (1) and (2).

At stage 6, LVEF, which is a function of

$$L V E {T}_{c}^{p} and {PEP}_{c}^{p} ,$$

is estimated at 761 using equation (7) or, (8).

At stage 7, the flow computes SV at 771 using equation (11), by measuring the first derivative of

$$B i o {Z}_{1}^{p} ( t )$$

to calculate base impedance

$${Z}_{0}^{p} and {( \partial {Z}_{c 1}^{p} / \partial t )}_{max} .$$

At stage 8, the flow computes ECF at 781 from R₀ using the algorithm as described by the equations (17)-(28).

The flow, in addition, computes right and left carotid pressure pulses using the raw bioimpedance waveforms ΔZ^{rc}(t) and ΔZ^{lc}(t) measured at 726 and 729 from a subject. Signal processing circuitry 790 is configured to extract physiological features from each cardiac cycle, including amplitude change in bioimpedance representing arterial blood volume change, inverse time difference between systolic and reflection waves serving as a proxy for pulse wave velocity, and beat-to-beat heart rate measurements. A neural network is configured to estimate systolic blood pressure, diastolic blood pressure, and pulse pressure values from said bioimpedance measurements using minimal training data. The neural network incorporates Taylor approximation-based physics constraints expressed as

$${P}_{i} ( {x}^{*} , {u}^{*} , θ ) = {f}_{NN} ( {x}_{i}^{*} , {u}_{i}^{*} , θ ) + \sum ( \partial {f}_{NN} / \partial {u}^{k} ) {\vert}_{i - th segment} ( {u}^{k} - {u}_{i}^{k} ) ,$$

where x* represents segmental carotid bioimpedance waveforms, u* represents extracted physiological features, and θ represents weights of the neural network, with the Taylor polynomial enabling gradual modeling of cardiovascular relationship changes. The neural network architecture may include a two-layer one-dimensional convolutional neural network with 32 and 64 filters, respectively, for processing the carotid bioimpedance waveforms ΔZ^{rc}(t) and ΔZ^{lc}(t) sampled at 30 Hz, max-pooling and flattening layers, feature concatenation means, and a two-layer fully connected network with 60 and 1 neurons. The neural network may be trained using a dual loss function which includes αL_{conventional}+βL_{physics}, where L_{conventional} represents standard supervised loss using labeled blood pressure data and L_{physics} represents physics-based loss from Taylor approximation residuals, with coefficients α=1 and β=10. The carotid bioimpedance signal processing includes application of second-order low-pass Butterworth filtering with 6 Hz cutoff frequency, automatic detection of cardiac cycle boundaries through maximum slope point identification in the first derivative signal, and extraction of nine fiducial points per cardiac cycle for feature calculation. The carotid artery location provides optimal signal quality due to its superficial position and direct branching from the aortic arch, enabling accurate detection of central arterial pulse characteristics with minimal tissue interference and strong pulsatile signal amplitude for continuous cardiovascular monitoring applications.

The flowchart demonstrates advanced signal processing, machine learning, and cardiovascular physics principles through the following key stages:

### Step 1: Signal Acquisition and Preprocessing

The process begins with raw bioimpedance signals ΔZ^{rc}(t) and ΔZ^{lc}(t) acquired from electrodes placed over the carotid artery region. The signals undergo second-order Butterworth low-pass filtering with 6 Hz cutoff frequency to remove high-frequency noise while preserving cardiac-related signal components.

### Step 2: Cardiac Cycle Segmentation

ECG R-wave detection provides timing references for cardiac cycle boundaries, while maximum slope point identification in the first derivative signal enables precise segmentation of individual heartbeats. This segmentation is crucial for extracting meaningful physiological features from each cardiac cycle.

### Step 3: Physiological Feature Extraction

Nine fiducial points are extracted from each cardiac cycle of the bioimpedance waveform ΔZ^{kc}(t) (k=R, L), to calculate the following three physiological features:
   (a) Change in amplitude (u₁)

$${u}_{1} = ( {Z}_{max} - {Z}_{min} ) / {Z}_{baseline} \qquad ( 30 a )$$

This represents the relative change in the amplitude of bioimpedance ΔZ^{kc}(t) corresponding to arterial blood volume variation during systolic expansion.
(b) Pulse wave velocity proxy (u₂)

$${u}_{2} = 1 / PTT = 1 / ( {t}_{reflection} - {t}_{systolic} ) \qquad ( 30 b )$$

where PTT is the pulse transit time calculated as the inverse time difference between systolic (t_{systolic}) and reflection (t_{reflection}) wave arrivals, serving as a proxy for carotid pulse wave velocity.
(c) Heart Rate Variability (u₃)

$${u}_{3} = {HR}_{beat - to - beat} = 60 / RR \qquad ( 30 c )$$

where beat-to-beat heart rate calculated from inter-beat intervals extracted from ΔZ^{kc}(t).

### Step 4: Neural Network Architecture Definition

(d) Input preparation

$${\vec{x}}_{i} = \{ {x}_{i 1} , {x}_{i 2} , … , {x}_{i n} \} : N - dimensional segmented bioimpedance waveform for {i}^{th} cardiac cycle \qquad ( 31 a )$$

$${\vec{u}}_{i} = \{ {u}_{i 1} , {u}_{i 2} , {u}_{i 3} \} : 3 - dimensional physiological feature vector . \qquad ( 31 b )$$

(e) Network structure
   The neural network function is defined as:

$${y}_{NN} = {f}_{NN} ( \vec{x} , \vec{u} , θ ) \qquad ( 32 )$$

where
   f_{NN}=Neural network approximation function,
   θ=Neural network weights and biases, and
   y_{NN}=Predicted carotid pressure output.
(f) Architecture components
   i. Convolutional Layers: Segmented bioimpedance beat z, is connected to a two-layer 1D CNN network—layer 1 with 32 filters, kernel size 5, activation ‘ReLU’, and layer 2 with 64 filters, kernel size 3, activation ‘ReLU’, with a max-pooling (pool size: 3, strides: 1) is applied to its output, followed by flattening.
ii. Feature Integration: A concatenation layer is used to combine flattened CNN outputs with u.
iii. Fully Connected Network (FCN) layers: Two dense layers—layer 1 with 60 neurons, activation ‘ReLU’, layer 2 with 1 neuron.

### Step 5: Physics-Informed Constraint Formulation

(g) Taylor approximation framework
   Using the feature vector \vec{u}, a polynomial with Taylor's approximation around i^{th} cardiac cycle is defined to model gradually changing cardiovascular relationships:

$${P}_{i} ( \vec{x} , \vec{u} , θ ) = {f}_{N N} ( {\vec{x}}_{i} , {\vec{u}}_{i} , θ ) + {\sum }_{k = 1}^{M} \frac{\partial {f}_{N N}}{\partial {u}^{k}} {\vert}_{i - th segment} ( {u}^{k} - {u}_{i}^{k} ) \qquad ( 33 )$$

where
   Pᵢ=Taylor polynomial approximated around i^{th} cardiac cycle,
M=Number of physiological features (M=3), and
∂f_{NN}/∂uₖ=Partial derivative calculated via automatic differentiation.
The output of this polynomial can be evaluated for any given \vec{x}, \vec{u} pair and neural network weights. We leverage this sequential nature of our data by evaluating Taylor polynomials approximated around i^{th} cycle for input values at (i+1)^{th} segment, i.e., Pᵢ(\vec{x}ᵢ₊₁, \vec{u}ᵢ₊₁, θ).
(h) Hemodynamic physics relationships
   The physics constraints embed known cardiovascular relationships:
      i. Volume-Pressure Dynamics:

$$Δ V ∝ Δ P \times {C}_{Arterial} \qquad ( 34 a )$$

where
ΔV: Changes in volume of carotid artery,
ΔP: Changes in blood pressure within the carotid artery, and
C_{Arterial}: Carotid arterial compliance i.e., ability of the carotid artery to expand and contract in response to changes in carotid pressure.
ii. Pulse Wave Velocity

$$( PWV ) = √ Eh / D ρ \qquad ( 34 b )$$

where
E: Elastic modulus of carotid artery (Pa),
h: Wall thickness of the carotid artery (m),
ρ: Average density of human blood (kg/m³), and
D: Diameter of the carotid artery in meters (m).

### Step 6: Automatic Differentiation Implementation

(i) Forward pass computation
   During forward propagation, the network computes

$${f}_{NN} ( {\vec{x}}_{i} , {\vec{u}}_{i} , θ ) \to pressure prediction \qquad ( 35 )$$

(j) Backward pass gradient computation
   Using automatic differentiation (backpropagation), partial derivatives are computed as:

$$\partial {f}_{N N} / \partial {u}_{1} = \partial {f}_{N N} / \partial ( change in amplitude ) , \qquad ( 36 a )$$

$$\partial {f}_{N N} / \partial {u}_{2} = \partial {f}_{N N} / \partial ( pulse wave velocity ) , and \qquad ( 36 b )$$

$$\partial {f}_{N N} / \partial {u}_{3} = \partial {f}_{N N} / \partial ( heart rate ) . \qquad ( 36 c )$$

### Step 7: Physics-Based Loss Function

(k) Residual Calculation
   For consecutive cardiac cycles, the physics-based residual is expressed as:

$${r}_{i} = {f}_{N N} ( {\vec{x}}_{i + 1} , {\vec{u}}_{i + 1} , θ ) - {P}_{i} ( {\vec{x}}_{i + 1} , {\vec{u}}_{i + 1} , θ ) . \qquad ( 37 )$$

(l) Total Loss Function

$${L}_{total} = α {L}_{conventional} + β {L}_{p h y s i c s} \qquad ( 38 )$$

$$where$$

$${L}_{conventional} = ( 1 / N ) {Σ}_{i} {\vert {y}_{true , i} - {f}_{N N} ( {\vec{x}}_{i} , {\vec{u}}_{i} , θ ) \vert}^{2} , \qquad ( 39 a )$$

$${L}_{p h y s i c s} = ( 1 / N ) {Σ}_{i} {\vert {r}_{i} \vert}^{2} , \qquad ( 39 b )$$

$$α = 1 ( conventional loss weight ) , and \qquad ( 39 c )$$

$$β = 10 ( physics loss weight ) . \qquad ( 39 d )$$

### Step 8: Training and Optimization

Gradient-Based Optimization
The network parameters are updated using:

$${θ}^{( t + 1 )} = {θ}^{( t )} - η \times ∇ Θ {L}_{total} \qquad ( 40 )$$

where η is the learning rate and ∇θLₜₒₜₐₗ represents gradients of the total loss with respect to network parameters.

### Step 9: Carotid Pressure Pulse Reconstruction

Calibration Process
The neural network output is calibrated using established cardiovascular relationships:

$$CAP ( t ) = {P}_{c a librated , N N} \times {f}_{N N} ( \vec{x} ( t ) , \vec{u} ( t ) , {θ}_{optimized} ) , and \qquad ( 41 )$$

$${P}_{calibrated , NN} = {P}_{c a librated} \times [ 1 + {α}_{1} \times impedance\_change + {α}_{2} \times PWV\_change + {α}_{3} \times HR\_change ] \qquad ( 42 )$$

where
   P_{calibrated} can be measured using ARTSENS pen, a non-invasive, portable, image free, ultrasound device developed by Indian Institute of technology, Madras (IIT-M) designed to assess arterial stiffness and central pressure pulse, and
   α₁, α₂, α₃=Physics-informed correction coefficients, and PWV_change, HR_change, impedance_change=Physiological parameter variations.

The flow also computes HR-corrected LVET, denoted as

$$LVE {T}_{c}^{r c}$$

at 791 and PTT-corrected PEP, denoted as

$$P E {P}_{c}^{r c}$$

at 792, using equations (3) and (4) for wing r.

Once carotid pulses at right (CAP^{rc}(t)) and left (CAP^{lc}(t)) carotid arteries are calculated at 793 and 794, respectively, LVEDP is obtained at 799 using equations (14)-(16) as described.

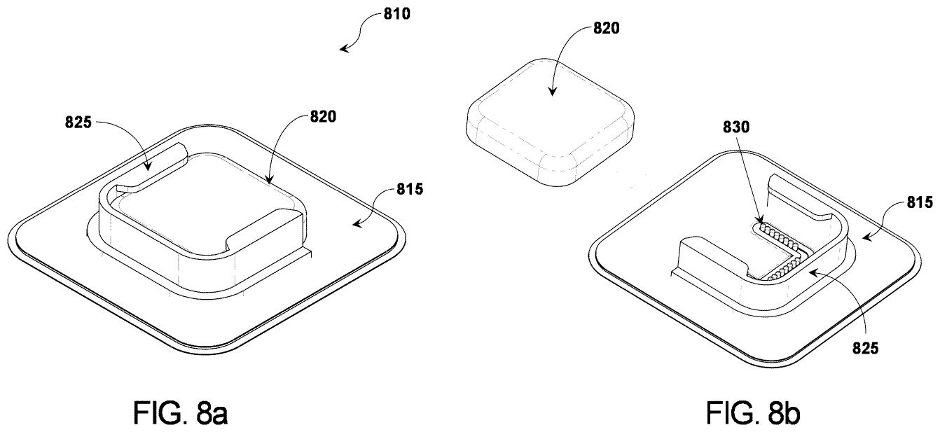

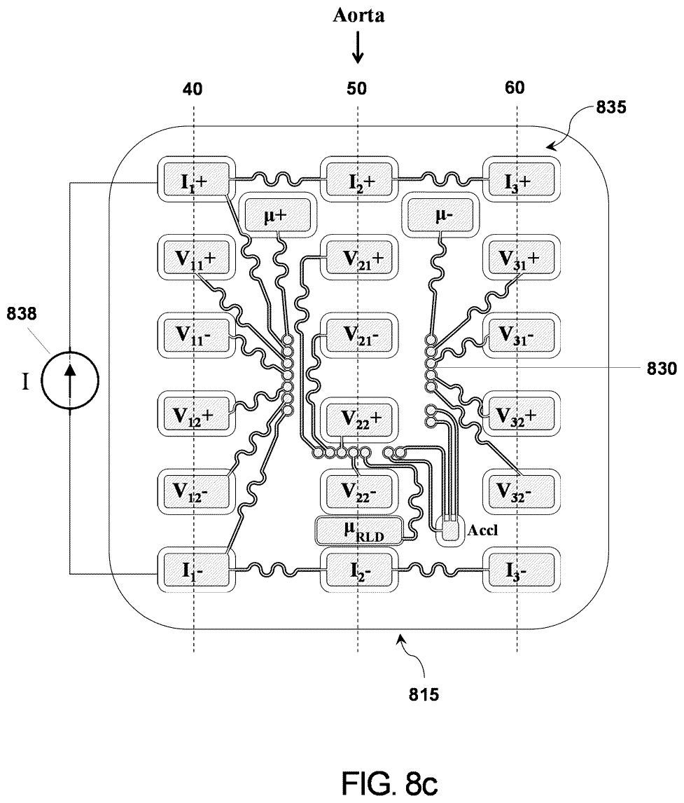

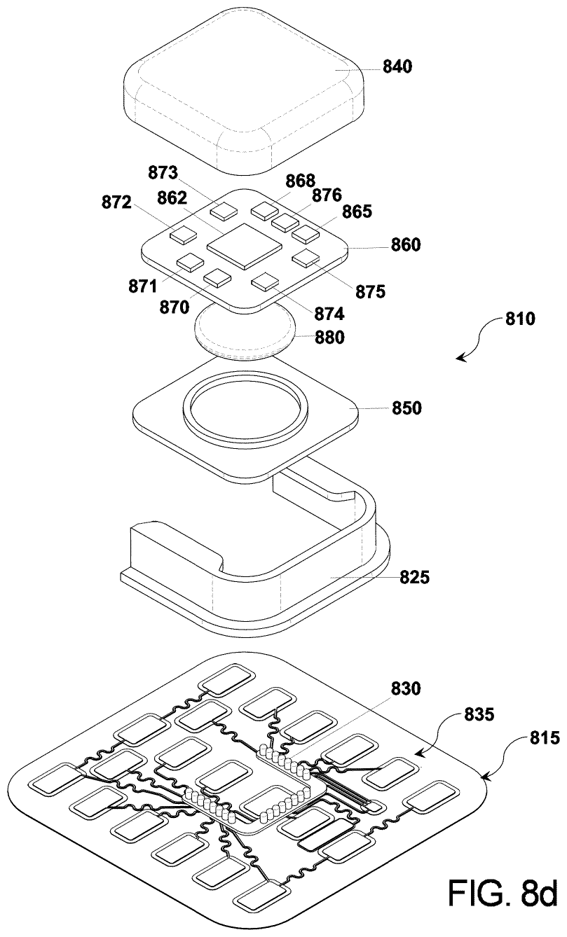

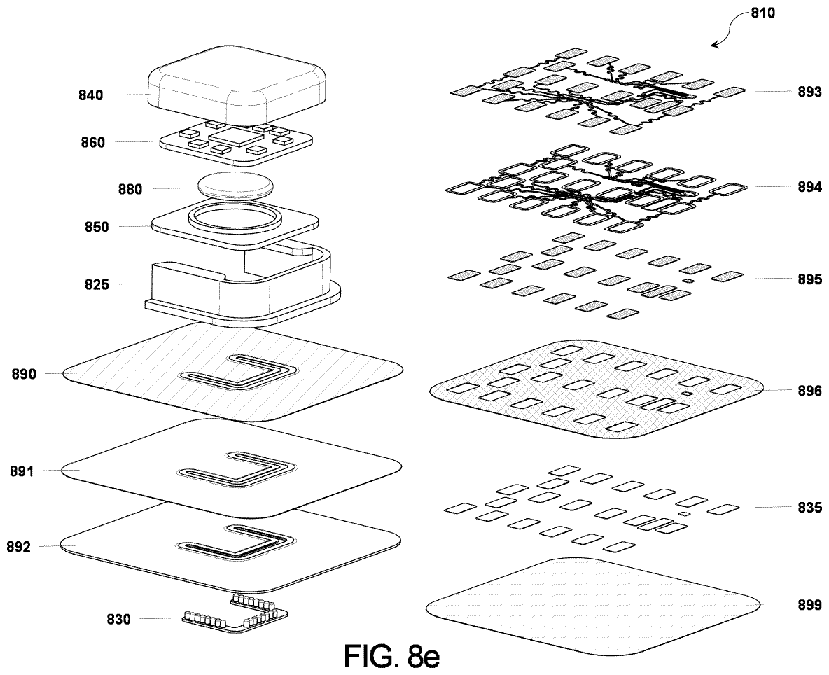

FIGS. 8a-8e shows various views of an embodiment of a chest-worn wearable hemodynamic monitoring device 810 based on bio-impedance (BioZ) sensors that monitor the pulsatile activity of the aorta and nearby arteries with high accuracy and reliability to provide high fidelity BP estimation.

FIG. 8a shows a 3D perspective view of an embodiment of a chest-worn wearable hemodynamic monitoring device 810. The device includes a flexible body 815 configured for placement over the chest around the aorta, a rigid housing 820, and a holder 825 configured to hold the rigid housing attached to the flexible substrate (body).

FIG. 8b shows an alternative 3D perspective view of the chest-worn wearable hemodynamic monitoring device 810. The device illustrates the overall form factor and integration of the rigid housing 820 with the flexible body 815 via magnetic Pogo pin connectors 830 for chest-worn deployment over the aortic region.

FIG. 8c shows a 2D perspective view of an embodiment of the flexible body 815 with an array of bioimpedance (BioZ) sensors configured within the wearable hemodynamic monitoring device. As shown, the array is a M×N (e.g., M=6, N≥3) electrode array arrangement designed to monitor pulsatile activity of the aorta from the chest surface, independent of precise sensing location. Other-sized arrays may also be useful.

FIG. 8d shows a 3D partially exploded view of an embodiment of the chest-worn wearable hemodynamic monitoring device 810, illustrating a multi-layered construction including the M×N array (M=6, N≥3) bioimpedance electrode array and 3 EKG electrodes with reference numerals indicating various structural and functional elements of the aortic monitoring assembly.

FIG. 8e shows a more detailed exploded 3D view of the chest-worn wearable hemodynamic monitoring device 810. As shown, the exploded 3D view includes a component breakdown of elements that constitute the flexible body and the rigid housing, demonstrating the modular construction of the chest-worn patch device for monitoring aortic pulsatile activity and cardiovascular events.

In one embodiment, the hemodynamic monitoring device 810 includes a flexible body 815 that conforms to the chest anatomy over the aortic region and a rigid housing 820 containing electronic components for signal processing and data transmission.

Referring to FIG. 8a, the chest-worn wearable hemodynamic monitoring device 810 is configured for continuous monitoring of aortic pulsatile activity and blood pressure estimation. The device 810 includes a flexible body 815 that serves as the primary substrate and interface with the patient's chest anatomy. The flexible body 815 is constructed from biocompatible materials such as medical-grade silicone or thermoplastic elastomer that provide adequate flexibility to conform to the natural curvature of the human chest while maintaining consistent electrode-to-skin contact throughout the monitoring period. The flexible body 815 incorporates, for example, a 6×3 bioimpedance electrode array having eighteen individual electrodes distributed in a rectangular matrix configuration, strategically positioned to optimize signal acquisition from the aortic region. Additionally, three EKG electrodes are integrated within the flexible body to provide simultaneous cardiac electrical activity monitoring for enhanced hemodynamic assessment and signal synchronization. Other array configurations may also be useful.

The flexible body contains a set of flexible serpentine traces that are embedded within the body and are mechanically isolated from the rigid housing. Each electrode trace is attached to the bottom surface of the flexible body 815 using a flexible electrode, and a sensor trace is connected to an accelerometer sensor on the same surface. This design enables the sensors and electrodes to make conformal contact with the user's skin, thereby facilitating the detection of both accelerometer and hemodynamic signals. The traces transmit electrical signals from the sensors and electrodes to the PCB assembly 860.

The device 810 further includes a rigid housing 820 that contains the electronic subsystems necessary for signal processing, data acquisition, and wireless communication. The rigid housing is fabricated from durable materials such as medical-grade plastics or lightweight metals that provide electromagnetic shielding and mechanical protection for the sensitive electronic components housed therein. These electronic components include but are not limited to bio-impedance signal conditioning circuits, EKG amplifiers, analog-to-digital converters, microprocessors for real-time signal processing, wireless communication modules, power management systems, and data storage capabilities. The rigid housing is dimensioned to accommodate these components while maintaining a low profile suitable for comfortable chest-worn deployment.

A key structural feature illustrated in FIG. 8a is the holder 825 configured to hold and attach the rigid housing 820 to the flexible substrate 815. The holder 825 is precisely engineered with dimensions that provide a secure mechanical coupling between the flexible substrate and the rigid electronics housing via a series of magnetic Pogo pin connectors 830 and mating pad connectors (not shown in figure) while preserving the overall flexibility characteristics of the device 810. This integration method ensures that the rigid housing 820 sits flush with the surface of the flexible body 815, creating a streamlined profile that minimizes patient discomfort and reduces the likelihood of the device catching on clothing or bedding during use. The design of the holder 825 also facilitates proper alignment of the electronic components within the rigid housing relative to the electrode array positioned in the flexible body, ensuring optimal signal acquisition performance.

FIG. 8b shows an alternative 3D perspective view of the wearable hemodynamic monitoring device, providing additional visual context for how the rigid housing get inserted to the holder 825 and gets attached to the flexible substrate 815. This perspective view emphasizes the ergonomic considerations incorporated into the design of the flexible body, particularly the contouring and surface topology that enables the device to conform to the chest anatomy in the region overlying the aorta. The flexible body exhibits curvature characteristics that match the natural chest wall geometry, ensuring that the bioimpedance electrode array maintains uniform contact with the skin surface across all the electrode positions. This uniform contact is critical for obtaining high-quality bio-impedance measurements that accurately reflect the pulsatile activity of the underlying aortic structure.

The perspective shown in FIG. 8b also illustrates the seamless integration between the flexible body 815 and the rigid housing 820, demonstrating a unified device appearance despite the modular construction approach. The rigid housing appears as an integral component of the overall device 810 rather than just an add-on element, which is important for patient acceptance and clinical usability. The low-profile design visible in this perspective ensures that the device may be worn continuously without interfering with normal patient activities such as sleeping, moving, or wearing clothing over the monitoring patch.

Furthermore, FIG. 8b provides insight into the spatial relationships between the various functional elements of the wearable device. The positioning of the rigid housing within the body is optimized to place the electronic processing components in close proximity to the center of the electrode array, thereby minimizing signal path lengths and reducing electromagnetic interference. This configuration also ensures that the wireless communication antenna, typically integrated within the rigid housing, is positioned for optimal signal transmission while maintaining the required separation from the bio-impedance measurement circuits to prevent interference.

Referring now to FIG. 8c, there is shown a comprehensive electrode array 835 embedded into the flexible body 815 of the chest-worn wearable hemodynamic monitoring device, illustrating how the various electrode elements and sensor components are interconnected through a sophisticated flexible trace network that terminates in magnetic Pogo pin connectors for interface with the rigid housing electronics 820. The electrode array configuration includes three distinct electrode columns designated as 40, 50, and 60, each incorporating multiple bioimpedance (BioZ) electrode elements arranged in a systematic pattern to provide enhanced signal quality and comprehensive spatial coverage of the aortic region within the upper thoracic cavity.

The bioimpedance electrode array, as discussed, includes three distinct electrode columns (40, 50, 60), each containing an array of six bioimpedance (BioZ) electrodes that require independent electrical connections to the signal acquisition unit. The current injection electrodes I₁+, I₂+, and I₃+ positioned at the top of each column are connected through dedicated conductive traces that route these signals to corresponding connection points within the magnetic Pogo pin interface system. Similarly, the negative current injection electrodes I₁−, I₂−, and I₃− located at the bottom of each column are connected through separate trace pathways to ensure proper current injection control and measurement isolation.

In one embodiment, first electrode column 40 includes a complete set of six bioimpedance electrodes strategically positioned along a first vertical axis to enable tetrapolar impedance measurements from the upper thoracic region. The column includes a first current-injecting electrode I₁+ positioned at the uppermost location of the column, which serves as the positive terminal for alternating current injection into the thoracic tissue. Correspondingly, a second current-injecting electrode I₁− is positioned at the lowermost location of column 40, serving as the negative terminal for current injection, thereby establishing a current field that penetrates the thoracic cavity and encompasses the aortic region of interest. Positioned intermediate to the current-injecting electrodes along column 40 are four voltage-sensing electrodes arranged in two differential pairs. A third electrode V₁₁+ is positioned below the positive current-injecting electrode I₁+, serving as the positive terminal of the first voltage-sensing pair for this column. A fourth electrode V₁₁− is positioned immediately below the third electrode V₁₁+, completing the first differential voltage-sensing pair (V₁₁+/V₁₁−) and enabling measurement of bioimpedance variations in the upper portion of the monitored thoracic region.

The second voltage-sensing pair within column 40 includes a fifth electrode V₁₂+ positioned below the fourth electrode V₁₁−, serving as the positive terminal of the second voltage-sensing pair. A sixth electrode V₁₂− is strategically positioned between the negative current-injecting electrode I₁− and the fifth electrode V₁₂+, completing the second differential voltage-sensing pair (V₁₂+/V₁₂−) and enabling measurement of bioimpedance variations in the lower portion of the monitored thoracic region along column 40.

In another embodiment, the second electrode column 50 incorporates an identical electrode configuration to column 40, including six bioimpedance electrodes arranged along a second vertical axis parallel to the first column. The column includes current-injecting electrodes I₂+ and I₂− positioned at the uppermost and lowermost locations, respectively, establishing a second current injection pathway through the thoracic tissue that provides spatial diversity for enhanced measurement reliability and coverage. The voltage-sensing configuration within column 50 mirrors that of column 40. For example, electrodes V₂₁+ and V₂₁− form the first differential voltage-sensing pair positioned in the upper region of the column, and electrodes V₂₂+ and V₂₂− form the second differential voltage-sensing pair positioned in the lower region of the column. This arrangement enables independent bioimpedance measurements from a second spatial location, providing redundancy and enhanced spatial resolution for aortic activity detection.

In the same embodiment, the third electrode column 60 completes the array configuration with an additional set of six bioimpedance electrodes arranged along a third vertical axis. The column includes current-injecting electrodes I₃+ and I₃− at the uppermost and lowermost positions, establishing a third current injection pathway that further enhances the spatial coverage and measurement diversity of the electrode array system. The voltage-sensing elements within the third column 60 includes electrodes V₃₁+ and V₃₁− which form the first differential pair, and electrodes V₃₂+ and V₃₂− which form the second differential pair, The configuration of the third column is consistent with the configuration employed in the first and second columns 40 and 50. This systematic electrode arrangement across all three columns provides a total of six independent voltage-sensing channels and three current injection pathways.

The voltage sensing electrodes within each column require individual trace connections to enable differential voltage measurements. In the first column 40, electrodes V₁₁+ and V₁₁− are connected through paired conductive traces that maintain signal integrity and minimize cross-talk, while electrodes V₁₂+ and V₁₂− are similarly connected through dedicated trace pairs. This pattern is replicated for the second and third columns 50 and 60, with electrode pairs (V₂₁+/V₂₁−, V₂₂+/V₂₂−) and (V₃₁+/V₃₁−, V₃₂+/V₃₂−), respectively, connected through their own isolated trace networks.

The electrode array 835 is supplied by a precision current source 838 designated as “I” in FIG. 8c, which provides controlled alternating current injection through the various electrode pairs. The current source is configured to deliver safe, controlled currents at frequencies optimized for bioimpedance measurement while maintaining patient safety in accordance with medical device standards for applied current limits.

The three-electrode ECG system shown in FIG. 8c includes μ+, μ−, and μ_{RLD} electrodes. The system requires specialized trace routing to ensure high-quality cardiac signal acquisition while maintaining electrical isolation from the bioimpedance measurement circuits. The positive ECG electrode μ+ is connected through a dedicated trace pathway that incorporates appropriate shielding and filtering characteristics to minimize interference from the bioimpedance current injection signals. The negative ECG electrode μ− is similarly connected through an independent trace that maintains the differential signal integrity required for high-quality ECG acquisition. The reference electrode μ_{RLD} is connected through a specialized trace network that provides the necessary common-mode rejection capabilities and driven-right-leg functionality essential for optimal ECG signal quality and patient electrical safety.

The accelerometer sensor, designated as “Accl” in FIG. 8c, is integrated within the flexible body 815 and requires multiple electrical connections to provide three-axis motion sensing capabilities. These connections are routed through dedicated traces within the flexible body to corresponding connection points in the magnetic Pogo pin interface. The accelerometer provides three-axis motion sensing capabilities that can be integrated with the ECG/EKG and bioimpedance signals to identify and correct for movement-induced measurement errors.

The conductive traces within the flexible body 815 may be implemented using advanced flexible printed circuit (FPC) technology that provides reliable electrical connectivity while maintaining the mechanical flexibility required for comfortable patient wear. The traces may be constructed using materials such as copper conductors with appropriate thickness and width specifications to handle the required current levels while minimizing electrical resistance and signal degradation. The trace routing incorporates electromagnetic compatibility (EMC) design principles including appropriate trace spacing, shielding where necessary, and ground plane implementation to minimize interference between the high-frequency bioimpedance signals and the low-frequency ECG measurements. Critical signal traces may incorporate differential pair routing techniques to enhance signal integrity and noise rejection capabilities.

The electrical connectivity between the flexible body 815 and the rigid housing 820 is accomplished through a magnetic Pogo pin connector system that provides reliable, low-resistance electrical contact while enabling easy assembly and disassembly of the device components. The flexible substrate incorporates spring-loaded magnetic Pogo pins 830 that correspond to each electrode and sensor connection, with these Pogo pins precisely positioned to align with the mating pad connectors integrated into a signal acquisition unit of the device. Each Pogo pin connector is electrically connected to its corresponding electrode or sensor through the flexible trace network, creating a complete electrical pathway from the physiological sensing elements to the analog front-end circuits within the rigid housing. The magnetic attraction force ensures consistent electrical contact between the Pogo pins and mating pads while accommodating minor positional variations during assembly.

The three-column electrode arrangement illustrated in FIG. 8_{lc} provides comprehensive spatial coverage of the aortic region through systematic positioning across the chest surface. The columnar organization enables multiple simultaneous measurements that can be processed using advanced signal processing algorithms to reconstruct high-fidelity representations of aortic pulsatile activity while suppressing interference from other physiological sources and environmental noise.

FIG. 8d shows a partially exploded view of the wearable hemodynamic monitoring device 810, which provides a comprehensive illustration of the individual components and sub-assemblies that are included in the integrated chest-worn monitoring system. This exploded view demonstrates the modular construction approach that enables efficient manufacturing, assembly, and potential serviceability of the device while maintaining the compact form factor required for comfortable patient wear.

The uppermost component in the exploded view is the upper housing member 840, which forms the top portion of the rigid housing assembly. The upper housing member 840 is constructed from durable, biocompatible materials such as medical-grade plastics or lightweight metal alloys that provide mechanical protection for the internal electronic components while maintaining electromagnetic compatibility requirements. The upper housing member 840 incorporates precision-molded features that facilitate secure connection to the lower housing member 850 through mechanical fastening mechanisms, snap-fit connections, and/or threaded interfaces that ensure reliable assembly while permitting disassembly for maintenance or component replacement when necessary.

Positioned centrally within the device assembly is the Signal Acquisition Unit (SAU) 860. The SAU may be implemented as a comprehensive printed circuit board (PCB) assembly that houses the primary electronic subsystems for bioimpedance measurement, ECG acquisition, signal processing, and wireless communication. In one embodiment, the SAU 860 incorporates multiple specialized analog front-end (AFE) circuits designated as 870, 871, 872, 873, 874, 875, and 876, each optimized for specific signal acquisition and conditioning functions.

The ECG/EKG analog front-end circuit 876 is specifically configured for acquiring cardiac electrical activity from the ECG electrodes μ+, μ−, and μ_{RLD} integrated within the flexible body 815. This AFE circuit 876 may be implemented using specialized integrated circuits such as the AFE4500 or AFE4960 from Texas Instruments, or alternatively the AD5940, AD5941, or MAX30001 from Analog Devices, providing high-resolution ECG signal acquisition with integrated filtering, amplification, and analog-to-digital conversion capabilities. The ECG AFE 876 generates digitized ECG waveforms suitable for real-time heart rate detection, rhythm analysis, and synchronization with bioimpedance measurements.

The bioimpedance measurement subsystem incorporates multiple AFE circuits (e.g., 870-875) that enable simultaneous multi-channel impedance measurements from the electrode array configuration. These AFE circuits are configured in pairs to support the three-column electrode arrangement, with each pair providing current injection and voltage sensing capabilities for independent bioimpedance measurements from different spatial locations across the chest surface.

The SAU 860 incorporates a central processing unit 862 that serves as the primary controller for all device functions, including signal acquisition coordination, real-time signal processing, data storage management, and wireless communication protocols. The processor 862 is configured as the SPI (Serial Peripheral Interface) master for coordinating communication with the multiple AFE circuits, utilizing individual Chip Select (CS) lines for each AFE to enable independent control and data acquisition from each measurement channel.

The processor 862 implements sophisticated bioimpedance measurement protocols that coordinate the timing and sequencing of current injection and voltage measurement across the multiple electrode columns. For example, during bioimpedance acquisition from the first electrode column 40, the processor 862 initiates current injection through AFE 870 via electrodes I₁+ and I₁− while simultaneously disabling current injection from AFE 871 by writing appropriate control commands to its configuration registers. This coordinated control prevents interference between measurement channels and ensures accurate impedance measurements.

The SAU 860 includes a dedicated memory unit 865 that provides both volatile and non-volatile storage capabilities for device operation. The memory unit 865 typically includes RAM for real-time signal processing operations, flash memory for firmware storage and configuration parameters, and potentially additional storage for buffering acquired physiological data prior to wireless transmission. The memory architecture is designed to support continuous data acquisition and processing while maintaining low power consumption characteristics essential for extended battery-powered operation.

Integrated within the SAU 860 is a wireless transmitter module 868 that enables real-time data transmission to external monitoring systems, mobile devices, or cloud-based healthcare platforms. The wireless module 868 may implement various communication protocols such as Bluetooth Low Energy (BLE), Wi-Fi, or cellular connectivity, depending on the specific application requirements and deployment scenarios. The module 868 is configured to transmit processed physiological parameters, raw signal data, and device status information while maintaining appropriate data security and patient privacy protections.

The device 810 includes a rechargeable battery 880 that provides the electrical power required for all device functions during extended monitoring periods. The battery 880 is typically implemented using lithium-ion or lithium-polymer technology to achieve high energy density in a compact form factor suitable for wearable applications. The power supply system includes integrated charging circuitry and power management functions that optimize battery life while ensuring reliable operation throughout the intended monitoring duration.

The exploded view in FIG. 8d illustrates the interconnection methodology between the flexible body 815 containing the electrode array 835 and the SAU 860. The connection is facilitated through the mating pad connectors (not shown) integrated into the SAU 860 that provide reliable electrical contact with the magnetic Pogo pin connectors 830. This connection system utilizes magnetic force to facilitate automatic attachment and detachment between the flexible body and the rigid housing, enabling easy assembly during manufacturing and potential field serviceability when required.

The lower housing member 850 forms the base portion of the rigid housing assembly and incorporates features necessary for secure integration with the flexible body 815. The lower housing member 850 includes precision-formed cavities and mounting features that accommodate the battery 880, provide access for charging connections, and ensure proper alignment of the mating pad connectors with Pogo pin connector interface 830.

The exploded view demonstrates how the various components stack together to form the complete device 810, with the flexible body 815 and its integrated electrode array 830 that forms the patient interface layer. The housing includes a lower housing member 850 and the holder 825 to provide the mechanical foundation, while the SAU 860 and battery 880 provide the functional electronics. The upper housing member 840 completes the protective enclosure of the components within the housing. This modular architecture enables efficient manufacturing processes while ensuring reliable long-term operation in clinical and home monitoring environments.

The overall design illustrated in FIG. 8d represents a sophisticated integration of flexible electrode technology, advanced signal processing electronics, and ergonomic packaging that collectively enable continuous, high-quality cardiovascular monitoring in a comfortable, wearable form factor suitable for extended patient use.

Referring to FIG. 8e, a more detailed exploded view of the wearable hemodynamic monitoring device 810 is shown. The detailed exploded view illustrate the component hierarchy and multi-layered construction that enables the integrated chest-worn cardiovascular monitoring capabilities. This exploded view demonstrates the sophisticated layered architecture that includes both the rigid housing assembly and the flexible body construction, revealing the individual components and their spatial relationships within the overall device structure.

As shown, the rigid housing 820 has an upper housing member 840, which can be readily attached to or detached from a lower housing member 850. This housing may incorporate a segment of the flexible body 815, a signal acquisition unit (SAU) 860, and a battery 880. The rigid housing is connected to the flexible body via a holder 825 that is precisely engineered with dimensions that provide a secure mechanical coupling between the flexible substrate and the rigid electronics housing via, for example, a series of magnetic Pogo pin connectors 830 and mating pad connectors while preserving the overall flexibility characteristics of the device 810. The flexible body 815 includes an upper substrate layer 890, a low-temperature hot melt adhesive layer 891, a lower substrate layer 892, a series of sensor and electrode trace layers 893, 894 and 895, an adhesive layer 896, an array of bioimpedance electrodes, a set of 3 ECG/EKG electrodes, and an accelerometer sensor 835. In addition, the flexible body includes a liner 899.

The uppermost layer of the flexible body 815 includes an upper substrate layer 890 constructed from elastomeric compression fabric that combines advanced textile materials such as polyester with LYCRA, nylon, or cotton-polyester rib construction. This layer provides the primary patient interface surface and incorporates stretchable characteristics that enable the device to conform to chest anatomy while maintaining consistent electrode-to-skin contact during patient movement and respiratory cycles. Alternative embodiments may utilize non-woven textile materials coated with biocompatible polymers such as polyurethane, nylon, or thermoplastic polyurethane (TPU) to achieve desired flexibility, durability, and skin compatibility characteristics. Other types of upper substrate layers may also be useful.

Positioned immediately below the upper substrate layer is a low-temperature hot melt adhesive layer 891 that provides secure bonding between the textile layers while maintaining flexibility characteristics. This adhesive layer utilizes thermoplastic double-sided adhesive film constructed from polyurethane materials that appear white and translucent, providing optimal bonding characteristics for textile and foam components. The adhesive formulation is specifically selected to remain non-sticky at room temperature while achieving strong bonding upon heating, yet maintaining stability across the expected operating temperature range of the wearable device. Alternative embodiments may employ TPU hot melt adhesive web materials that provide similar bonding characteristics while ensuring long-term adhesion stability and biocompatibility for extended patient contact applications.

The lower substrate layer 892 provides structural support for the conductive trace network and electrode elements while maintaining the flexibility characteristics required for comfortable patient wear. This layer, in one embodiment, is constructed from materials compatible with the printing and deposition processes used for the conductive trace formation, ensuring reliable adhesion and electrical performance of the embedded circuitry. Other configurations of lower substrate layers may also be useful.

A connector assembly was provided in which both substrate layers 890 and 892, along with the intermediate hot melt adhesive layer 891, were configured with a precision-formed groove of equal size, enabling the penetration of magnetic Pogo pin connectors 830 through the layers to establish electrical contact with mating pad connectors positioned within the lower housing 850; the groove also functioned to ensure proper alignment of the Pogo pin connectors with the corresponding connector interface, thereby facilitating secure and accurate engagement in accordance with design specifications.

The electrical connectivity within the flexible body is accomplished, in one embodiment, through a sophisticated three-layer conductive trace system that provides isolated pathways for the multiple electrode connections while maintaining signal integrity and preventing electrical interference between measurement channels. The three-layer conductive trace system includes a first and second conductive trace layers 893 and 895 separated by an isolation layer 894. Other configurations of a conductive trace system may also be useful.

In one embodiment, the first conductive trace layer 893 includes stretchable silver (Ag) ink materials that provide low-resistance electrical pathways for the bioimpedance and ECG electrode connections. The silver ink formulation is specifically selected to maintain electrical conductivity during mechanical flexion and stretching associated with device deployment and patient movement.

As for the intermediate dielectric layer 894, it provides electrical isolation between the conductive trace layers while incorporating precisely positioned openings that enable controlled interconnections between the upper and lower conductive layers. This dielectric layer utilizes specialized ink formulations that provide reliable electrical insulation while maintaining flexibility characteristics. The dielectric layer serves the critical function of preventing silver migration between conductive traces, thereby eliminating the risk of short circuits that could compromise device functionality or patient safety.

In one embodiment, the second conductive trace layer 895 includes silver chloride (AgCl) ink materials that provide specialized electrical characteristics particularly beneficial for ECG electrode applications. The AgCl formulation provides stable electrode-electrolyte interface characteristics that minimize polarization effects and baseline drift commonly associated with extended ECG monitoring applications. Alternative embodiments of the conductive trace system may utilize conductive textile materials such as stretchable fabric incorporating materials like Shieldex® “P130+B” or similar conductive textile technologies that provide electrical connectivity while maintaining superior mechanical flexibility and durability characteristics compared to printed ink systems. Additional embodiments may implement the conductive traces using advanced flexible printed circuit (FPC) technology that provides reliable electrical connectivity through conventional copper conductor materials while maintaining the mechanical flexibility required for comfortable patient wear. Such FPC implementations utilize materials and construction techniques specifically designed for dynamic flexing applications.

In one embodiment, a specialized adhesive layer 896 with empty spaces corresponding to the electrodes and sensors provides protection for the printed conductive traces and sensing elements while facilitating secure bonding between the various substrate layers. This layer utilizes biocompatible adhesive formulations that ensure long-term adhesion stability while maintaining the flexibility characteristics essential for device performance. In an embodiment, the adhesive material can be made of ARcare® 93690, ARcare® A-5490 or materials with similar properties.

The flexible body incorporates a comprehensive array of sensing elements, including the BioZ electrode array configured in the three-column arrangement (columns 40, 50, 60), the three-electrode ECG system (μ+, μ−, μ_{RLD}), and the integrated accelerometer sensor. These sensing elements are positioned within the adhesive layer 896 and the release liner 899 with precise spatial relationships that optimize signal acquisition while maintaining the flexibility and comfort characteristics required for extended patient monitoring.

The bottommost layer may include a patient interface liner 899 that provides the direct skin contact surface for the wearable device. This liner incorporates biocompatible materials selected for optimal skin compatibility, moisture management, and long-term comfort during extended monitoring periods. The liner may incorporate specialized surface treatments or coatings that enhance electrode-skin interface characteristics while preventing skin irritation or allergic reactions.

The exploded view in FIG. 8e demonstrates how the multiple layers and components are integrated to form a unified wearable device that combines sophisticated electronic functionality with comfortable and flexible construction suitable for extended chest-worn deployment. The layered architecture enables efficient manufacturing processes while ensuring reliable long-term performance in clinical and home monitoring environments. The modular construction approach illustrated in the exploded view facilitates quality control during manufacturing, enables component-level testing and validation, and provides potential serviceability advantages for field maintenance when required. The integration of rigid electronic components with flexible substrate technology represents a significant advancement in wearable medical device design, addressing the competing requirements of electronic sophistication and patient comfort in a single unified system.

The operational principle of the wearable monitor in FIG. 8a is similar to that of the wearable monitor in FIG. 5a. Three adjacent columns 40, 50 and 60 of the electrode arrays were utilized for BioZ sensing to measure 6 impedances (Zᵢⱼ=ΔVᵢⱼ/I; i, j∈1(1)3) from different sensing locations around the aorta. The AC current waveform of amplitude I was injected in three columns between the electrodes at the top and bottom rows to provide the best current distribution for the three columns. The six bioimpedance signals were measured from the middle pairs in the three columns with 2 voltage measurements per column, as shown in FIG. 8c and are expressed as:

For column 40:

$${Z}_{11} = ( {V}_{11} + - {V}_{{11}^{-}} ) / ( {I}_{1} + - {I}_{1} - ) = Δ {V}_{11} / I \qquad ( 43 a )$$

$${Z}_{12} = ( {V}_{12} + - {V}_{12} - ) / ( {I}_{1} + - {I}_{1} - ) = Δ {V}_{12} / I \qquad ( 43 b )$$

For column 50:

$${Z}_{21} = ( {V}_{21} + - {V}_{21} - ) / ( {I}_{2} + - {I}_{2} - ) = Δ {V}_{21} / I \qquad ( 43 c )$$

$${Z}_{22} = ( {V}_{22} + - {V}_{22} - ) / ( {I}_{2} + - {I}_{2} - ) = Δ {V}_{22} / I \qquad ( 43 d )$$

For column 60:

$${Z}_{31} = ( {V}_{31} + - {V}_{31} - ) / ( {I}_{3} + - {I}_{3} - ) = Δ {V}_{31} / I \qquad ( 43 e )$$

$${Z}_{32} = ( {V}_{32} + - {V}_{32} - ) / ( {I}_{3} + - {I}_{3} - ) = A {V}_{32} / I \qquad ( 43 f )$$

In one embodiment, upon transmission of real-time waveforms to a web-based server via an Internet of Things (IoT) gateway, a deep-learning model is activated to forecast the waveforms several days to months in advance. The forecasted waveforms enable determination of pulse arrival time (PEP), pulse transit time (PTT), and left ventricular ejection time (LVET) within the pulmonary region. An average PTT measurement at the pulmonary region is utilized to calculate a PTT-corrected pulse arrival time, referred to as PEP_{c}. Likewise, an average LVET measurement in the pulmonary region is used to calculate a heart rate-corrected LVET, referred to as LVET_{c}. The PTT measurement is further employed in the calculation of pulse wave velocity (PWV) and associated arterial pressure, while PEP_{c} and LVET_{c} are used to calculate left ventricular ejection fraction (LVEF) and left ventricular end-diastolic pressure (LVEDP) concurrently. Continuous and diligent monitoring of these cardiovascular parameters enables early detection of potential heart failure events, thereby facilitating preemption of pulmonary edema onset and reducing hospitalization rates.

In various embodiments, a system and method are provided for processing physiological signals collected from a wearable device. The method may include a multistage data processing pipeline or “flow,” which enables systematic acquisition, analysis, and transformation of signals into clinically and operationally relevant parameters.

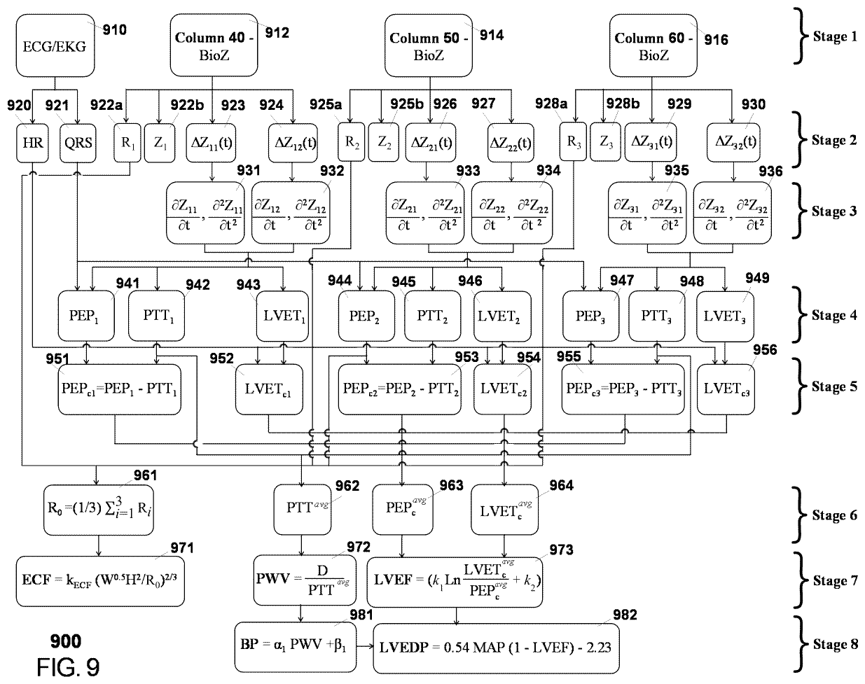

FIG. 9 shows an embodiment of a multistage flow 900. The flow enables systematic acquisition, analysis, and transformation of signals into clinically and operationally relevant parameters.

At stage 1, physiological data from the wearable biosensing device is acquired. This stage may be referred to herein as the “input stage.” In one embodiment, various analog or digital signal streams are received from electrode arrays coupled to the user.

More specifically:
EKG/ECG data is acquired via electrodes μ+, μ−, and μ_{RLD}, as shown at 910, permitting derivation of time-domain cardiac activity.
Impedance plethysmography (IPG) data from column 40 is acquired via differential leads I₁+ and I₁−, represented as Z₁₁(V₁₁+, V₁₁−) and Z₁₂(V₁₂+, V₁₂−) at 912.
Similarly, additional impedance signals from column 50, obtained via the I₂+ and I₂-electrodes are received as Z₂₁(V₂₁+, V₂₁−) and Z₂₂(V₂₂+, V₂₂−) at 914, and those from column 60, via electrodes I₃+ and I₃−, appear as Z₃₁(V₃₁+, V₃₁−) and Z₃₂(V₃₂+, V₃₂−) at 916.

In an exemplary embodiment, input data at 910 is used to compute one or more physiological parameters, including, but not limited to QRS complex duration (QRSd), Heart rate (HR) and Heart rate variability (HRV).

The impedance channels Z₁₁ and Z₁₂ collected at 912 are used to quantify variations in thoracic bioimpedance associated with respiratory and cardiac cycles. These signals can be characterized as follows:

$${Z}_{11} = {Z}_{1} + Δ {Z}_{11} and {Z}_{12} = {Z}_{1} + Δ {Z}_{12} \qquad ( 44 a )$$

where
Z₁ denotes the non-time-varying (DC) base impedance, corresponding to static anatomical structures including muscle, bone, and fat, and
ΔZ₁₁ and ΔZ₁₂ denote time-varying (AC) components attributable to physiological functions such as respiration and cardiac output.

The impedance channels Z₂₁ and Z₂₂ collected at 914 are used to quantify variations in thoracic bioimpedance associated with respiratory and cardiac cycles. These signals can be characterized as follows:

$${Z}_{21} = {Z}_{2} + Δ {Z}_{21} and {Z}_{22} = {Z}_{2} + Δ {Z}_{22} \qquad ( 44 b )$$

where
Z₂ denotes the non-time-varying (DC) base impedance, corresponding to static anatomical structures including muscle, bone, and fat, and
ΔZ₂₁ and ΔZ₂₂ denote time-varying (AC) components attributable to physiological functions such as respiration and cardiac output.

The impedance channels Z₃₁ and Z₃₂ collected at 916 are used to quantify variations in thoracic bioimpedance associated with respiratory and cardiac cycles. These signals can be characterized as follows:

$${Z}_{31} = {Z}_{3} + Δ {Z}_{31} and {Z}_{32} = {Z}_{3} + Δ {Z}_{32} \qquad ( 44 c )$$

where
Z₃ denotes the non-time-varying (DC) base impedance, corresponding to static anatomical structures including muscle, bone, and fat, and
ΔZ₃₁ and ΔZ₃₂ denote time-varying (AC) components attributable to physiological functions such as respiration and cardiac output.

To facilitate the acquisition of Z₁₁ and Z₁₂, an AC excitation current in the range of approximately 100 μA to 400 μA is applied between the I₁+ and I₁− electrodes. The excitation frequency may be selected within the range of 50 kHz to 100 kHz.

To isolate and analyze the physiological contributions to ΔZ₁₁ and ΔZ₁₂ from column 40, the following signal processing operations may be performed:

### Respiratory Component Extraction:

Signals Z₁₁ and Z₁₂ are first filtered using a low-pass finite impulse response (FIR) filter at 10 Hz, which eliminates high-frequency noise.
The output is further processed using a second-stage interpolated low-pass FIR filter at 0.5 Hz, optimizing isolation of respiration-induced oscillations.

### Cardiac Component Extraction:

Signals Z₁₁ and Z₁₂ are detrended using a linear detrend function followed by a zero-mean normalization.
The detrended signals are passed through a band-pass FIR filter, with default cutoff frequencies set to 0.9 Hz (low-pass) and 7 Hz (high-pass), providing temporal isolation of cardiac-induced impedance changes. These cutoff parameters may be adaptively adjusted in accordance with signal quality metrics or characteristics obtained via the bioimpedance AFE(s).

Once respiratory components are suppressed, the residual signal Z₁₁(t) can be modeled as a sum of a fixed DC baseline component Z₁ and a dynamic time-varying component ΔZ₁₁(t), the latter being primarily cardiogenic in origin.

A similar procedure is implemented to facilitate the acquisition of ΔZ₂₁ and ΔZ₂₂ from column 50 and ΔZ₃₁ and ΔZ₃₂ from column 60.

At stage 2, the input data is used to calculate heart rate (HR) at 920, QRS duration (QRS_{d}) at 921, R₁ at 922a, Z₁ at 922b, ΔZ₁₁(t) at 923, ΔZ₁₂(t) at 924, R₂ at 925a, Z₂ at 925b, ΔZ₂₁(t) at 926, ΔZ₂₂(t) at 927, R₃ at 928a, Z₃ at 928b, ΔZ₃₁(t) at 929, ΔZ₃₂(t) at 930. Other parameters may also be determined at stage 2. It is appreciated that other physiological and electrical parameters may also be computed at this stage based on available signal input, processing resources, or clinical relevance.

In one embodiment, extracellular fluid (ECF) measurement is performed using Cole-Cole modeling techniques, wherein the impedance parameters R₁, R₂, and R₃ are extracted from three sets of BioZ electrode arrays, each corresponding, for example, to an electrode column denoted as: the first column for R₁ at 922a (e.g., column 40 in FIG. 8c), the second column for R₂ at 925a (e.g., column 50 in FIG. 8c), and the third column (e.g., column 60 in FIG. 8c) for R₃ at 928a.

Each Cole-Cole resistance (R₁, R₂, and R₃) is determined using a time-domain model-fitting algorithm, based on single-frequency sinusoidal excitation signals that are DC-biased. The excitation is applied across high-frequency current injection pairs along each electrode column. The frequency and amplitude of such signals may be chosen to optimize tissue discrimination and improve signal-to-noise performance.

The algorithm used to estimate the Cole-Cole parameters performs regression fitting across sampled impedance curves and utilizes optimization techniques such as nonlinear least squares estimation (NLSE) or Levenberg-Marquardt solvers. The impedance signal is decomposed into its resistive and capacitive components to isolate the extracellular and intracellular fluid contributions.

Additional details on the algorithm implementation and equations have previously been provided. For example, the previously provided algorithm implementation describes the multi-step process for stable and interpretable parameter extraction, allowing accurate estimation of ECF levels under varying physiological conditions.

In one embodiment, at stage 3 of the flow, the system is configured to compute the first-time and second-time derivatives of impedance waveforms obtained from multiple electrode columns, wherein such computations are performed at a defined time t=T₀. Specifically, at 931, ∂Z₁₁/∂t (i.e., first derivative of Z₁₁(t) with time t) and ∂²Z₁₁/∂t² (i.e., 2^{nd} derivative of Z₁₁(t) with time t) are computed from Z₁ signal; at 932, ∂Z₁₂/∂t (i.e., first derivative of Z₁₂(t) with time t) and ∂²Z₁₂/∂t² (i.e., 2^{nd} derivative of Z₁₂(t) with time t) are computed from Z₁₂ signal; at 933, the system computes ∂Z₂₁/∂t (i.e., first derivative of Z₂₁(t) with time t) and ∂²Z₁₂/∂t² (i.e., 2^{nd} derivative of Z₁₂(t) with time t); at 934, ∂Z₂₂(t)/∂t (i.e., first derivative of Z₂₂(t) with time t) and ∂²Z₂₂/∂t² (i.e., 2^{nd} derivative of Z₂₂(t) with time t) are evaluated from Z₂₂ signal; at 935, the corresponding derivatives of Z₃₁, including ∂Z₃₁/∂t and ∂²Z₃₁/∂t², are determined; and at 936, the flow computes ∂Z₃₂/∂t and ∂²Z₃₂/∂t².

In one embodiment, at stage 4 of the flow, the system evaluates systolic time intervals for each wing of the wearable device, including pre-ejection period (PEP), pulse transit time (PTT), and left ventricular ejection time (LVET).

Specifically, PEP₁ is computed at 941 as the time delay from the onset of the QRS complex—or alternatively, a ventricular pacing stimulus—to the initiation of central aortic flow, represented by the B point on the first derivative of thoracic impedance. Similarly, PEP₂ is computed at 944 as the time delay from QRS onset or pacing pulse to the onset of right carotid arterial flow, as identified on the derivative of the BioZ signal from the corresponding lateral electrode column, and PEP₃ is computed at 947 as the delay from QRS onset or pacing pulse to the onset of left carotid arterial flow. These PEP values offer non-invasive insights into electromechanical coupling and ventricular contractile timing.

In one embodiment, for the BioZ electrode array placed along the first column (e.g., column 40 in FIG. 8c), pulse transit time PTT₁ is computed at 942 based on the relative time delay between ΔZ₁₁(t) and ΔZ₁₂(t) waveforms evaluated at three distinct points along their respective descending slopes: a beginning delay point (Tₙ), a middle delay point (Tₘ), and an end delay point (Tₙ). In this configuration, Tₘ corresponds to the time delay between maximum descending slope (MDS) points of ΔZ₁₁(t) and ΔZ₁₂(t), while Tₙ and Tₚ correspond to the relative timing between negative and positive extrema, respectively, of the second derivatives ∂²Z₁₁/∂t² and ∂²Z₁₂/∂t². Likewise, a similar estimation is performed for the BioZ electrode array across the second column (e.g., column 50 in FIG. 8c), where PTT₂ is computed at 945 based on the delay between ΔZ₂₁(t) and ΔZ₂₂(t), with the same three evaluation points: Tₙ, Tₘ at the MDS point, and Tₚ, again derived from the second derivatives ∂²Z₂₁/∂t² and ∂²Z₂₂/∂t². For the third column (e.g., column 60 in FIG. 8c), PTT₃ is computed at 948 from the time delay between ΔZ₃₁(t) and ΔZ₃₂(t) using the same methodology, where Tₘ reflects the MDS-based delay, and Tₙ and Tₚ correspond to time differences between the locations of the negative and positive peaks of the second derivatives ∂²Z₃₁/∂t² and ∂²Z₃₂/∂t², respectively. This approach enables accurate estimation of segmental PTT values which can be used for vascular health assessment or for calculating derived indices such as pulse wave velocity.

In one embodiment, LVET is calculated as the temporal distance between the B point, which marks the onset of ventricular ejection, and the X point, which signifies the end of ejection, on the differentiated impedance signal ∂Z/∂t. For the electrode array positioned along the first column (e.g., column 40 in FIG. 8c), LVET₁ is computed at 943 as the average time interval between the B and X points on the first derivatives of the Δ Z₁₁(t) and ΔZ₁₂(t) signals. Similarly, LVET₂ is computed at 946 from the average interval between the B and X points on the differentiated signals ∂Z₂₁/∂t and ∂Z₂₂/∂t, corresponding to the second column (e.g., column 50 in FIG. 8c). Likewise, for the third column (e.g., column 60 in FIG. 8c), LVET₃ is computed at 949 as the averaged interval between the B and X points on ∂Z₃₁/∂t and ∂Z₃₂/∂t. These LVET values are indicative of cardiac contractile performance and may be employed for non-invasive hemodynamic monitoring.

In one embodiment, at stage 5 of the process flow, the system is configured to compute corrected systolic timing intervals for each of the three thoracic bioimpedance columns, wherein both the pre-ejection period (PEP) and the left ventricular ejection time (LVET) are adjusted to account for confounding cardiovascular dynamics, specifically heart rate (HR) and pulse transit time (PTT). For the bioimpedance electrode array positioned along the first column (e.g., column 40 in FIG. 8c), the PTT-corrected pre-ejection period, denoted as PEP₁, is computed at 951, and the HR-corrected left ventricular ejection time, denoted as LVET₁, is computed at 952. Similarly, for the second column array (e.g., column 50 in FIG. 8c), PEP₂ and LVET₂ are computed at 953 and 954, respectively, using analogous correction algorithms. For the third column (e.g., column 60 in FIG. 8c), the system calculates the PTT-adjusted pre-ejection period PEP₃ at 955 and the HR-adjusted ejection time LVET₃ at 956. These corrected timing intervals provide increased physiological interpretability and enable improved tracking of electromechanical cardiac performance across all regions of thoracic monitoring, supporting differential analysis of central and peripheral arterial dynamics under varying cardiovascular loads.

Since three adjacent columns of the electrode array were utilized for BioZ sensing to measure 6 BioZ channels from different sensing locations around the aorta, at stage 6 of the flow, the system evaluates R₀ at 961, which is the mean of the Cole-Cole resistances (R₁, R₂, and R₃) calculated at stage 2. Similarly, an average of the systolic time intervals, i.e., PTT^{avg} is calculated at 962, an average of PTT-corrected pre-ejection period (PEP), i.e.,

$$P E {P}_{c}^{avg}$$

is calculated at 963 and an average of HR-corrected left ventricular ejection time, i.e., LVET_{c3}, is calculated at 964.

In one embodiment, at stage 6 of the process flow, the system is configured to evaluate composite hemodynamic parameters by utilizing data obtained from three adjacent columns of the electrode array positioned around the aorta for BioZ sensing, thereby measuring six distinct BioZ channels from different anatomical locations. At this stage, a mean Cole-Cole resistance value, R₀, is computed at 961 as the arithmetic mean of the Cole-Cole resistances R₁, R₂, and R₃ previously calculated at stage 2, providing an aggregate index of thoracic tissue resistance properties. Concurrently, the system computes at 962 the average pulse transit time, PTT^{avg} by averaging the segmental PTT measurements derived from each electrode column; similarly, at 963, an average PTT-corrected pre-ejection period,

$${PEP}_{c}^{avg} ,$$

is calculated from the PEP values corrected for individual PTT across all sensing regions. At 964, the average heart rate-corrected left ventricular ejection time,

$${LVET}_{c}^{avg} ,$$

is determined from the LVET values that have been individually normalized for heart rate effects along each thoracic column. These spatially averaged metrics improve the robustness of physiological interpretation by mitigating local signal variations and provide comprehensive indicators of global cardiovascular function suitable for trending and clinical assessment.

In one embodiment, at stage 7 of the process flow, the system is programmed to compute the extracellular fluid (ECF) volume at 971, wherein the computation is based on the mean Cole-Cole resistance parameter R₀. In parallel, the system calculates aortic pulse wave velocity (PWV) at 972 using time-of-flight techniques that measure signal propagation delay between upstream and downstream impedance channels surrounding the aorta. Additionally, the system estimates the LVEF at 973, wherein, in one embodiment, the estimation follows Chirfle's formula, which maps impedance-derived systolic time intervals and possibly stroke volume estimates to an inferred ejection fraction value. These computations collectively provide integrated cardiovascular metrics that reflect both fluid compartment status and cardiac mechanical performance, enabling longitudinal monitoring of global cardiac function through fully non-invasive means.

In one embodiment, at stage 8 of the flow, the system is configured to estimate arterial pressure (BP) at 981 as a mathematical function of the aortic pulse wave velocity (PWV) computed in the preceding stage, wherein established physiological relationships between arterial stiffness and pressure are employed to derive non-invasive blood pressure measurements from the PWV. Subsequently, the system calculates LVEDP at 982 using the clinically validated Abd-El-Aziz equation, specifically:

$$LVEDP = 0.54 \times MABP \times ( 1 - LVEF ) - 2.23 \qquad ( 45 )$$

where
MABP is the Mean arterial blood pressure i.e., MABP=(SBP+2×DBP)/3,
SBP and DBP are Systolic and Diastolic blood pressure derived from the PWV-based arterial pressure estimation, and
LVEF is the Left ventricular Ejection Fraction computed at stage 7.
   This equation has been found to provide the most accurate, reliable, and easily implemented method for non-invasive LVEDP estimation, particularly in patients with HFpEF and LVEDP values below 20 mmHg. The combination of PWV-derived arterial pressure and bioimpedance-derived ejection fraction enables continuous, non-invasive monitoring of left ventricular filling pressure, which is a critical hemodynamic parameter for cardiovascular health assessment and heart failure management.

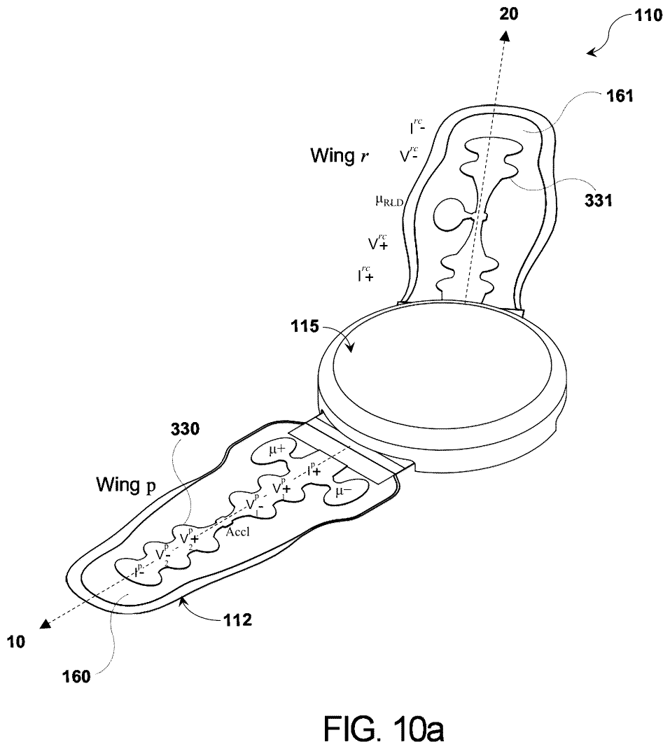

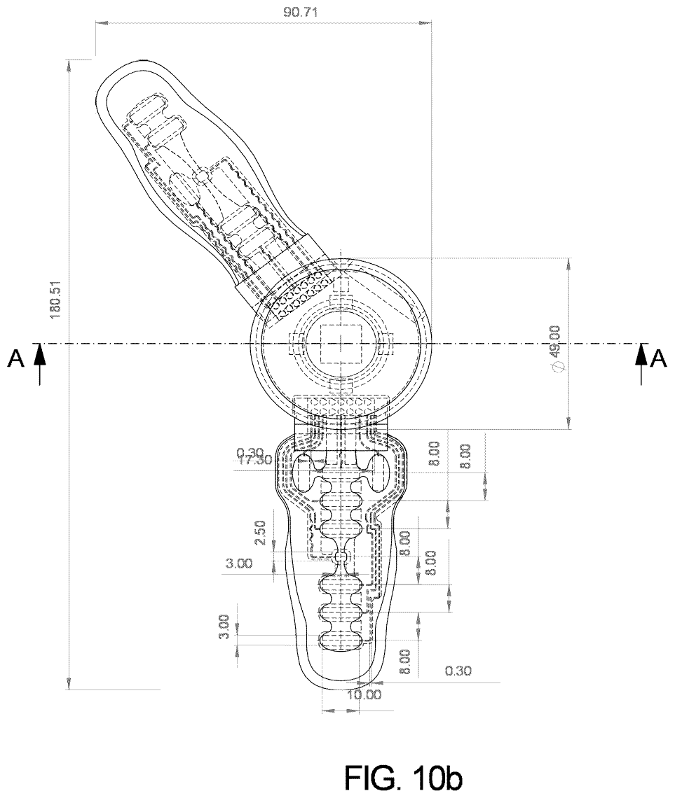

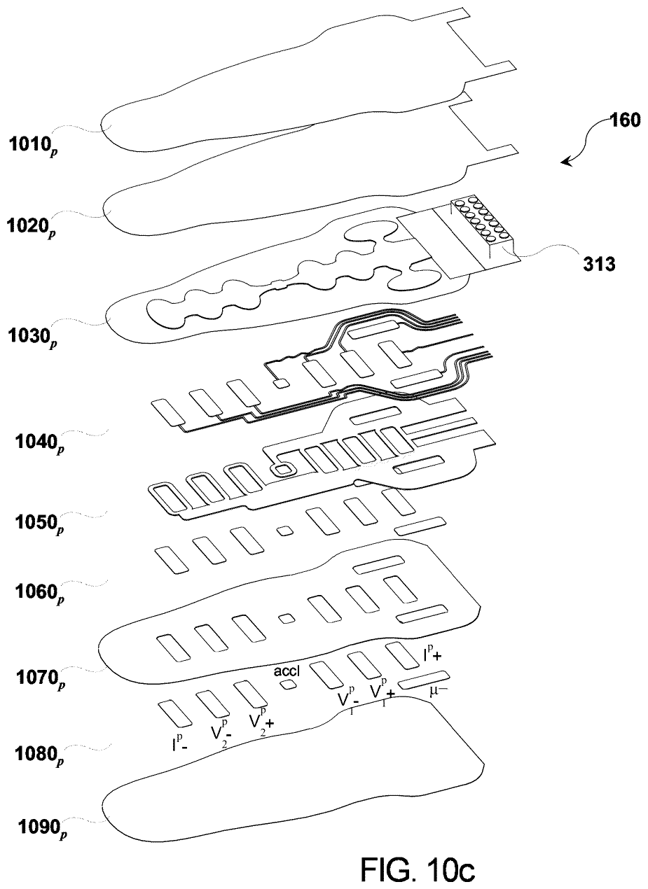

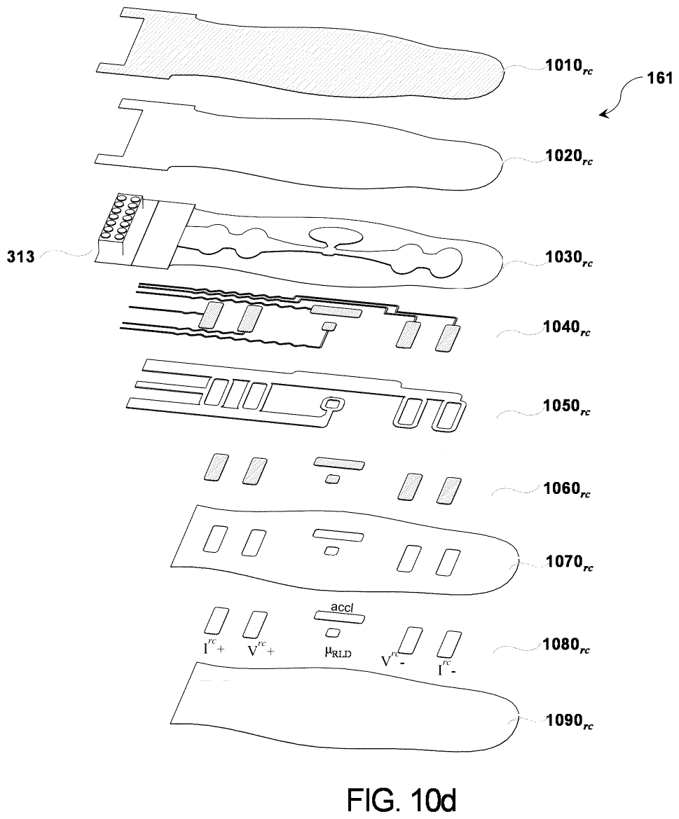

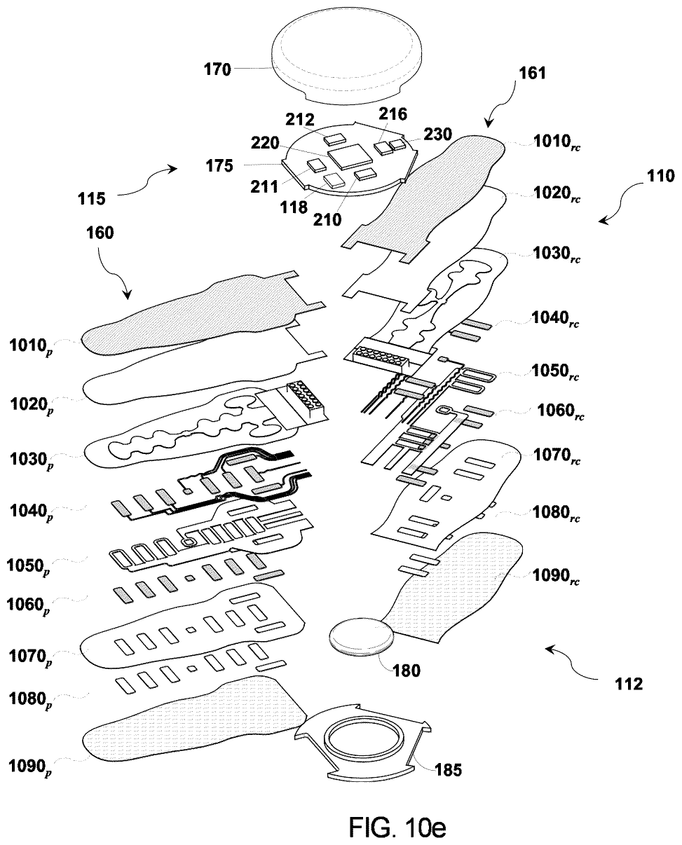

FIGS. 10a-10ₑ illustrate various views and exploded assemblies of an embodiment of a wearable hemodynamic monitoring device 110 configured for continuous acquisition of hemodynamic, electrophysiological, and motion signals in a user.

FIG. 10a provides a 3D perspective view of a hemodynamic monitoring device 110 which includes a flexible body 112 operably coupled to a water-tight and rigid housing 115. The flexible body 112 is contoured to conform to the anatomical surface of a user and includes a first bioimpedance measuring wing 160 adapted for placement on the midsternal region of the upper thorax and a second bioimpedance measuring wing 161 configured for application to the right side of the neck over the right common carotid artery (RCCA). The flexible body 112 further incorporates ultrathin, stretchable substrate layers 330 and 331 that extend laterally from the rigid housing 115 to under the wings. Each of wings 160 and 161 includes a plurality of embedded flexible serpentine traces, said traces being mechanically isolated from the rigid housing 115; wherein individual electrode traces are affixed to the lower surface of flexible body 112 by flexible electrodes and sensor traces are electrically connected to accelerometer sensors disposed on the same surface. This configuration enables the respective sensors and electrodes to form conformal contact with the user's skin for reliable detection of accelerometer and hemodynamic signals. The serpentine traces are operatively connected to transmit electrical signals from the sensors and electrodes to a printed circuit board (PCB) assembly 175 positioned within the rigid housing 115. Each of the wings 160 and 161 is mechanically secured to the rigid housing 115 using either a magnetic mating pad connector 313 or an alternative flexible interconnection technique. The rigid housing 115 is further provided with an onboard power source, which may include one or more batteries 180.

In one embodiment, flexible wings 160 and 161 are arranged to encompass electrode arrays configured to detect ECG/EKG, bioimpedance, and accelerometer waveforms, enabling acquisition of both atrial and ventricular cardiac activity. The sensor arrangement also facilitates monitoring of hemodynamic activity in the descending aorta and lung filling cycles. In this embodiment, the primary bioimpedance measuring wing 160 includes a first electrode (I^{p}+) and a second electrode (I^{p}−) positioned along the midsternal line 10 on the upper and lower aspects thereof, respectively; these electrodes are configured to provide current injection into the upper thoracic region of the user. A third electrode

$$( {V}_{1}^{p} + )$$

and a fourth electrode

$$( {V}_{1}^{p} - )$$

are further disposed along the same midsternal line, with the third electrode positioned inferior to the first current-injecting electrode (I^{p}+) and the fourth electrode positioned inferior to the third electrode. These third and fourth electrodes are arranged to detect voltage at specified locations on the upper thoracic region.

Additionally, a fifth electrode

$$( {V}_{2}^{p} + )$$

and a sixth electrode

$$( {V}_{2}^{p} - )$$

are positioned along the midsternal line, with the fifth electrode located inferior to the fourth electrode and the sixth electrode positioned between the second current-injecting electrode (I^{p}−) and the fifth electrode; these electrodes detect voltage at a different upper thoracic location.

An accelerometer sensor is placed between the fourth

$$( {V}_{1}^{p} - )$$

and fifth

$$( {V}_{2}^{p} + )$$

electrodes and is capable of detecting three-axis motion signals. A pair of electrodes (μ+, μ−) are disposed along a line orthogonal to the midsternal line 10 flanking the first current-injecting electrode (I^{p}+) on either side. The electrodes (μ+, μ−), along with a reference electrode R_{LD} disposed in wing 161, are configured for ECG/EKG signal detection.

Correspondingly, the neck-worn wing 161 includes a second array of electrodes, including a first electrode (I^{rc}+) and a second electrode (I^{rc}−) positioned proximate the right common carotid artery (RCCA) 20 at upper and lower aspects of wing 161, respectively, and are configured for current injection into the user's right neck. Further, a third electrode (V^{rc}+) and a fourth electrode (V^{rc}−) are positioned along the RCCA 20, with the third electrode disposed inferior to the first current-carrying electrode (I^{rc}+) and the fourth electrode disposed superior to the second current-carrying electrode (I^{rc}−). These third and fourth electrodes detect voltage at specified locations on the RCCA region.

FIG. 10b shows a 2D cross-sectional view along line A-A, illustrating the structural integration between the rigid circular housing 115 and the bioimpedance wings 160 and 161. The housing 115 includes an internal compartment adapted to house the electronic circuits and sensor interfaces. The wings 160, and 161 include flexible substrates that are dimensioned and contoured for anatomical regions. In one embodiment, the wings are dimensioned and contoured for the midsternal thoracic region and the right side of the neck (over the right common carotid artery). In one embodiment, the basal width of each wing is approximately 10 mm, narrowing down to about 8 mm along the longitudinal direction. Such a configuration optimizes both adherence and flexibility. Internal reinforcement layers and sensor channels are indicated by broken lines, which delineate pathways for the embedded sensor traces and electrode conductors. A central aperture is formed in the housing 115 for coupling with mounting components or electrical connectors. Overall, the device exhibits maximum lateral and vertical dimensions of about 90.71 mm and 180.51 mm, respectively. The internal channel diameters for routing electrical conductors are shown as 3.00 mm, 2.50 mm, and 0.30 mm, and passageways within the wings enable reliable electrical and mechanical connections between flexible and rigid subsystems.

FIG. 10c depicts a 3D exploded view of an embodiment of the bioimpedance measuring wing 160. FIG. 10c details the multilayer composition of wing 160, revealing successive layers including: a top fabric layer 1010ₚ formed from elastomeric compression fabric (such as polyester-LYCRA blend, nylon, or coated nonwoven), a low-temperature hot melt adhesive film 1020ₚ for securing the fabric to the underlying substrate 1030ₚ (constructed from flexible, stretchable polymers including TPU and polyurethane), and a stack of sensor and electrode trace layers 1040ₚ, 1050ₚ, and 1060ₚ. The trace layers may include stretchable conductive ink (e.g., Ag or AgCl) or conductive textile traces, with openings in a dielectric ink layer for electrical connection. An adhesive layer 1070ₚ is provided beneath the conductive trace stack, with voids to accommodate a first array of six bioimpedance and two ECG electrodes, as well as an accelerometer sensor 1080ₚ. A removable release liner 1090ₚ protects the adhesive before application. The flexible electrodes are affixed to the skin-contact side using hydrogel or hydrogel-based blends. The described multilayer stack enables conformal contact, durable adhesion, and precise sensing on the user's thoracic region.

FIG. 10d shows a 3D exploded view of an embodiment of the bioimpedance measuring wing 161. The construction is substantially analogous to that of wing 160, with corresponding top fabric layer 1010_{rc}, low-temperature hot melt adhesive film 1020_{rc}, substrate 1030_{rc}, and an array of three sensor and electrode trace layers 1040_{rc}, 1050_{rc}, and 1060_{rc}, followed by an adhesive layer 1070_{rc}. A corresponding set of bioimpedance, ECG/EKG electrodes, and an accelerometer sensor 1080_{rc} are included, with each layer designed for robust mechanical and electrical integration. The liner 1090_{rc} facilitates correct placement and application of the neck-worn wing. The electrode and sensor placement in wing 161 is optimized for anatomical registration with the right common carotid artery, with electrode traces specifically designed to capture hemodynamic activity and motion signals from the neck region.

FIG. 10e illustrates an exploded view of the hemodynamic monitoring device 110, showing components that form the flexible body 112 and rigid housing 115. The rigid housing 115 includes an upper housing member 170 and a lower housing member 185, configured to be readily attached and detached. The housing assembly is adapted to enclose a segment of the flexible body 112, a signal acquisition unit (SAU) 175, a battery 180, and spring connectors (not shown), referred to as magnetic Pogo Pins. The spring connectors, as discussed, facilitate electrical connections. In one embodiment, the flexible body 112 includes first and second bioimpedance measuring wings 160 and 161. The first bioimpedance measuring wing includes two current-injection electrodes and four voltage detection electrodes positioned on the chest. The second bioimpedance measuring wing 161 includes two current-injection and two voltage detection electrodes, configured for neck placement.

Detailed layer construction of wing 160 includes a top fabric layer 1010ₚ, a low-temperature hot melt adhesive layer 1020ₚ, a TPU substrate layer 1030ₚ, a series of sensor and electrode trace layers 1040ₚ, 1050ₚ, and 1060ₚ, an adhesive layer 1070ₚ, an array of six bioimpedance electrodes, two ECG/EKG electrodes, an accelerometer sensor 1080ₚ, and a liner 1090ₚ. Similarly, wing 161 includes a top fabric layer 1010_{rc}, a low-temperature hot melt adhesive layer 1020_{rc}, a TPU substrate layer 1030_{rc}, sensor and electrode trace layers 1040_{rc}, 1050_{rc}, and 1060_{rc}, an adhesive layer 1070_{rc}, a flexible electrode array, an accelerometer sensor 1080_{rc}, and a liner 1090_{rc}.

FIG. 10e further depicts the rigid housing 115, where the upper housing member 170 is removably connected to the lower housing member 185, enclosing several components, including a portion of the flexible body 112, an SAU 175 in the form of a printed circuit board (PCB) assembly, battery 180, and spring connectors. The SAU 175 is positioned within the housing 115 to establish electrical contact with sensors, electrode traces, and the battery 180 via spring connectors and mating pad connectors on both the PCB and flexible trace sides, respectively. SAU 175 may include multiple analog front-end (AFE) circuits, namely AFEs 210, 211, 212, and 216, a processor 220, memory 230, a wireless transmitter module 118, and a power supply 180.

The embodiments of FIGS. 10a-e are similar to those of FIGS. 2a-d and 5a-e. For example, the rigid 115 housing may be the same. However, for FIGS. 10a-e, only two connectors connect wings 160 and 161 to the rigid housing. In other embodiments, the rigid housing may be configured with two connectors for connecting to wings 160 and 161. In addition, the wings 160 and 161 may also be similar to those described in FIGS. 2a-d and 5a-e. This highlights the flexibility of the modular design of the monitoring device.

The device 110 is configured such that wing 160, when attached to the upper thoracic (midsternal) region, and wing 161, when attached to the right side of the neck over the right common carotid artery, may both inject electrical current and detect voltage at specified locations. In various embodiments, the embedded sensors and electrode arrays are optimized and spatially distributed to enable concurrent measurement of atrial and ventricular electrical activity, thoracic bioimpedance, and body motion. In use, a first array within wing 160 includes electrodes for current injection (I^{p}+, I^{p}−) and separate electrodes

$$( {V}_{1}^{p} + , {V}_{1}^{p} - , {V}_{2}^{p} , {V}_{2}^{p} - )$$

for detecting voltage at staggered positions to facilitate regional hemodynamic measurement. The neck-worn wing 161 includes current-injecting electrodes (I^{rc}+, I^{rc}−) and voltage-detecting electrodes (V^{rc}+, V^{rc}−) for carotid arterial bioimpedance acquisition, and all wings may incorporate accelerometer sensors for capturing kinetic signals. Additional electrodes (μ+, μ−, μ_{RLD}) are positioned for optimal ECG/EKG acquisition.

In one embodiment, the bioimpedance measurement process for wing 160 initiates with the initialization of a Serial Peripheral Interface (SPI) communication protocol, wherein the microcontroller is programmed as the SPI master. Separate chip select (CS) lines are assigned to AFE(s) 210 and 211, thereby enabling independent control of each AFE over the SPI bus. An excitation current pulse is output from AFE 210 at time t=T₀ and delivered to the user's upper thoracic region via electrodes I^{p}+ and I^{p}−. Simultaneously, current injection from AFE 211 is disabled by writing to its control register through selection of AFE 211 via its CS line, issuance of the disable command, and subsequent deselection. Consequently, AFE 210 operates in a dual capacity to provide both current injection and voltage measurement, while AFE 211 is configured exclusively for voltage measurement. AFE 210 outputs a clock signal to AFE 211 to synchronize the measurement cycles of both devices, as configured by SPI commands from the microcontroller. This synchronization results in a trigger signal that initiates simultaneous measurement, allowing AFE 210 to provide excitation current while both AFEs concurrently capture voltage responses, yielding precise and correlated data. Bioimpedance data is then read out from both AFE 210 and AFE 211 for further processing.

In another embodiment, the excitation current waveform generated by the bioimpedance AFE 212 is injected into the right common carotid artery of the user via I^{rc}+ and I^{rc}− electrodes in wing 161. The processor 220 is configured to preprocess the input data from the AFEs, the wireless transmitter module 118, and the accelerometer sensors. The wireless module 118 communicates wirelessly with a smartphone 120 and a web server 140 using Bluetooth, Wi-Fi, or other wireless communication standards. The preprocessed data may be forwarded to the user's mobile device for further interpretation by the wireless transmission module. In addition, the preprocessed data may be stored in the memory unit as needed.

The operational principle of the wearable monitor in FIG. 10a is the same as that of the wearable monitor described in FIG. 5a. The signal processing unit (SAU) 175 is programmed to simultaneously activate the wings 160 and 161. This enables remote monitoring of deteriorating hemodynamic parameters in heart failure patients.

Once the wearable monitoring device 110 is activated, real-time impedance waveforms are generated simultaneously for each wing:

For wing p:

$${BioZ}_{1}^{p} = ( {V}_{1}^{p} + - {V}_{1}^{p} - ) / ( {I}^{p} + - {I}^{p} - ) \qquad ( 46 a )$$

$${BioZ}_{2}^{p} = ( {V}_{2}^{p} + - {V}_{2}^{p} - ) / ( {I}^{p} + - {I}^{p} - ) \qquad ( 46 b )$$

For wing r:

$${BioZ}^{rc} = ( {V}^{rc} + - {V}^{rc} - ) / ( {I}^{rc} + - {I}^{rc} - ) \qquad ( 46 c )$$

In one embodiment, the device further acquires real-time ECG/EKG data and three-axis accelerometer waveforms. The real-time waveforms are transmitted to a web-based server through an Internet of Things (IoT) gateway, whereupon a deep learning model is activated to forecast these waveforms several days to months in advance. The forecasted bioimpedancesignals

$${BioZ}_{1}^{p} and {BioZ}_{2}^{p}$$

enable measurement of ECF, LVEF, SV, and CO within the pulmonary region. To compute right carotid pressure pulses from raw bioimpedance waveforms BioZ^{rc}, the device includes signal processing circuitry configured to extract physiological features from each cardiac cycle, including amplitude changes in bioimpedance indicative of arterial blood volume variations, inverse time differences between systolic and reflection waves as a proxy for pulse wave velocity, and beat-to-beat heart rate measurements. A neural network processing unit is configured to estimate systolic blood pressure, diastolic blood pressure, and pulse pressure values based on the bioimpedance measurements using a minimal training dataset. The neural network incorporates physics-based constraints employing a Taylor approximation, expressed as

$${P}_{i} ( {x}^{*} , {u}^{*} , θ ) = {f}_{NN} ( {x}_{i}^{*} , {u}_{i}^{*} , θ ) + \sum ( \partial {f}_{NN} / \partial {u}^{k} ) {\vert}_{i - th segment} ( {u}^{k} - {u}_{i}^{k} ) ,$$

wherein x* represents segmental carotid bioimpedance waveforms, u* represents extracted physiological features, and θ represents neural network weights, enabling gradual modeling of cardiovascular relationship changes.

The architecture includes a two-layer one-dimensional convolutional neural network with 32 and 64 filters for processing carotid bioimpedance waveforms sampled at 30 Hz, max-pooling and flattening layers, feature concatenation means, and a two-layer fully connected network with 60 and 1 neurons. The neural network is trained using a dual loss function αL_{conventional}+βL_{physics}, where L_{conventional} is conventional supervised loss using labeled blood pressure data, and L_{physics} is physics-based loss from Taylor approximation residuals, with a and R coefficients of 1 and 10, respectively. Bioimpedance signal processing further includes second-order low-pass Butterworth filtering with a cutoff frequency of 6 Hz, automatic cardiac cycle boundary detection by identifying maximum slope points in the first derivative signal, and extraction of nine fiducial points per cardiac cycle for feature calculation. The choice of the carotid artery location provides optimal signal quality due to its superficial anatomical position and direct branching from the aortic arch, which allows accurate detection of central arterial pulse characteristics with minimal tissue interference and strong pulsatile amplitude for continuous cardiovascular monitoring applications. Once the carotid pulse CAP^{rc}(t) across the right carotid artery is calculated, LVEDP is computed using equations (24)-(26).

In one embodiment, clinical parameter extraction from the wearable patch described in FIG. 10a is performed using a software flow 700 configured to process data recorded by sensors and electrodes of the wearable device, similar to the device depicted in FIGS. 5a-5e. In one embodiment, the preprocessed data acquired from the wearable device is transmitted to a user's mobile device, which subsequently forwards the data to a cloud server. Alternatively, the preprocessed data may be directly transmitted from the wearable device to the cloud server. The main printed circuit board (PCB) assembly of the wearable device may include a mobile communication module, such as Wi-Fi or Bluetooth Low Energy (BLE), enabling wireless transmission of preprocessed data to the cloud server via cellular or other wireless networks. This data transmission and processing flow may be managed by a software application executing on the cloud server. Other transmission techniques or configurations may also be useful.

The disclosed wearable bioimpedance system provides significant technical advantages over existing cardiac monitoring solutions through its dual phenotype detection capability, wherein both pulmonary intravascular congestion (ALVEDP) and tissue congestion (AECF) are simultaneously monitored and assessed. Unlike existing solutions that are limited to single-parameter monitoring across implantable, point-of-care, or wearable platforms, the present invention enables enhanced diagnostic accuracy through the concurrent measurement of multiple congestion phenotypes, thereby reducing the likelihood of misdiagnosis or delayed detection of cardiac decompensation events. Early intervention capabilities are facilitated through predictive algorithms that process the dual phenotype data to identify cardiac decompensation patterns before clinical manifestation, allowing for timely care plan development and therapeutic intervention. Multi-sensor intelligence is employed to assess a plurality of biomarkers (5+ clinical parameters) simultaneously, which significantly reduces the probability of false positive alerts that plague single-parameter monitoring systems. The wearable form factor provides scalability advantages over implantable and point-of-care solutions, enabling deployment across a broad disease spectrum, including atherosclerosis, acute coronary syndrome, cardiac ischemia, and acute myocardial infarction, wherein the non-invasive monitoring approach eliminates the procedural risks and costs associated with implantable devices while providing superior continuous monitoring compared to intermittent point-of-care assessments.

The disclosed system incorporates a neural network (NN) architecture that demonstrates superior data efficiency compared to conventional machine learning approaches, requiring only 4-10% labeled training data versus 75-88% for traditional methods while achieving clinical-grade accuracy for both systolic and diastolic pressure estimation, meeting AAMI Grade A compliance standards. The neural network implementation utilizes Taylor approximation-based physics constraints, enabling personalized baseline modeling with minimal calibration requirements and real-time processing capabilities. Competing wearable technologies such as biobeat and nanowear lack the capability for direct LVEDP estimation and ECF assessment simultaneously, while traditional bioimpedance devices including Bodystat, ImpediMed and NiCAS are limited to static body composition analysis without dynamic cardiovascular parameter extraction or predictive analytics integration.

The technical advantages of the disclosed system extend to its multi-parameter integration capabilities, combining LVEDP monitoring through carotid bioimpedance analysis, ECF assessment via Cole-Cole parameter extraction (R₀), and cardiovascular risk stratification through AI-powered data fusion algorithms. This integrated approach was configured to address the unmet clinical need for concurrent, continuous, and non-invasive assessment of multiple congestion phenotypes, including pulmonary intravascular and extravascular, that existing technologies have been unable to monitor comprehensively within a single platform.

LVEDP serves as a powerful predictor of cardiovascular outcomes and overall mortality, while bioimpedance-derived parameters including ECF status provide comprehensive insights into cellular health and biological aging. The integration of these measurements into a continuous, wearable monitoring platform enables real-time assessment of health status and early detection of physiological deterioration.

The scalability of the disclosed wearable patch can be demonstrated through its extended application to the monitoring of acute myocardial infarction (AMI) and atherosclerosis, in addition to its traditional use in heart failure management. The non-invasive monitoring of left ventricular end-diastolic pressure (LVEDP) and cardiac output (CO) has been enabled by the patch, allowing for the assessment of physiologic parameters recognized as predictors of adverse clinical outcomes. In patients with AMI—particularly those presenting with NSTEMI—elevated LVEDP exceeding 22 mmHg has been associated with increased in-hospital heart failure and mortality, using thresholds validated by sensitivity and specificity analyses. Real-time hemodynamic tracking has been facilitated by the patch, supporting early intervention strategies and therapeutic guidance. Furthermore, the measurement of cardiac output—correlated against direct Fick method benchmarks with high concordance—has been incorporated to enable assessment of atherosclerosis progression and systemic cardiac function. By being designed for continuous, remote data acquisition in non-clinical environments, the device has been positioned to offer scalable, data-driven cardiovascular monitoring across a broader range of diseases and patient populations.

As the disclosed technology continues to evolve and gain clinical validation, its potential to revolutionize cardiovascular care and extend healthy lifespan becomes increasingly apparent. The integration of artificial intelligence, machine learning, and advanced bioimpedance analysis creates a powerful platform for precision medicine and personalized longevity interventions. With proper clinical validation, regulatory approval, and healthcare system integration, the presently disclosed wearable monitoring technology has the potential to significantly impact population health outcomes and contribute to meaningful extensions of healthy human lifespan.

The future of longevity assessment lies in the seamless integration of advanced monitoring technologies with personalized healthcare interventions. The presently disclosed approach to wearable LVEDP and ECF monitoring represents a crucial step toward this vision, offering the promise of earlier disease detection, more effective treatments, and ultimately, longer, healthier lives for patients worldwide.

In the preceding sections, the invention has been described with specific exemplary embodiments and examples thereof. It is evident, however, that various modifications may be made without departing from the broader spirit and scope of the invention, as outlined in the following claims. Therefore, the specification and drawings are to be regarded in an illustrative manner rather than a restrictive one. The scope of the invention is thus indicated by the appended claims, rather than by the foregoing description, and all changes that come within the meaning and range of equivalency of the claims are intended to be embraced therein.

1. A wearable hemodynamic monitoring device for monitoring left ventricular (LV) parameters of a subject comprising:
a rigid housing with a signal acquisition unit (SAU), the SAU is configured with a data transmission module and a processing module;
a flexible body which includes bioimpedance and ECG/EKG electrodes and accelerometer sensors configured to acquire raw bioimpedance, EKG/ECG and acceleration waveforms from a midsternal region and a common carotid artery region of a subject wearing the hemodynamic monitoring device, the flexible body is connected to the rigid housing and are in electrical communication with the SAU;
wherein the processing module of the SAU preprocesses the raw bioimpedance, EKG/ECG and acceleration waveforms acquired from the subject to produce preprocessed bioimpedance, EKG/ECG and acceleration waveforms;
wherein the data transmission module transmits the preprocessed biopedance, EKG/ECG and acceleration waveforms to a backend system for analysis; and
wherein the wearable hemodynamic monitoring device facilitates predicting heart failure based on multiple simultaneous congestion phenotypes.

2. The wearable hemodynamic monitoring device of claim 1, wherein:
the flexible body comprises elongated flexible wings with the bioimpedance and EKG/ECG electrodes; and
the elongated flexible wings extend from the rigid housing.

3. The wearable hemodynamic monitoring device of claim 2, wherein the elongated flexible wings are configured to be connectable to and detachable from the rigid housing.

4. The wearable hemodynamic monitoring device of claim 2, wherein the flexible body comprises first and second elongated flexible wings, wherein:
the first wing is configured to position the bioimpedance and EKG/ECG electrodes of the first wing along the midsternal of the subject;
the second wing is configured to position the bioimpedance and EKG/ECG electrodes along a side of the subject's neck over a common carotid artery of the side.

5. The wearable hemodynamic monitoring device of claim 4, wherein the one side of the subject's neck is the right side of the subject's neck over a right common carotid artery overlying the sternocleidomastoid muscles.

6. The wearable hemodynamic monitoring device of claim 4, wherein
the first wing further comprises a first accelerometer sensor; and
the second wing further comprises a second accelerometer sensor.

7. The wearable hemodynamic monitoring device of claim 2, wherein the flexible body comprises first, second and third elongated flexible wings, wherein:
the first wing is configured to position the bioimpedance and EKG/ECG electrodes of the first wing along a midsternal of a subject;
the second wing is configured to position the bioimpedance and EKG/ECG electrodes along a right side of the subject's neck on a right common carotid artery overlying the sternocleidomastoid muscles; and
the third wing is configured to position the bioimpedance and EKG/ECG electrodes along a left side of the subject's neck on a left common carotid artery overlying the sternocleidomastoid muscles.

8. The wearable hemodynamic monitoring device of claim 7, wherein
the first wing further comprises a first accelerometer sensor;
the second wing further comprises a second accelerometer sensor; and
the third wing further comprises a third accelerometer sensor.

9. The wearable hemodynamic monitoring device of claim 2, wherein the flexible body comprises:
an array of rows and columns of bioimpedance electrodes; and
EKG/ECG electrodes and accelerometer sensor disposed within rows of the array of bioimpedance electrodes.

10. The wearable hemodynamic monitoring device of claim 9, wherein the flexible body comprises a rectangular-shaped body disposed on a subject's chest around the aorta.
